SEQUENCE LISTING (228 sequences)

SEQ ID NO: 1 - protein, 10 aa, synthetic construct
  REGION 1..10: note = Synthetic peptide
GGPQGIWGQG

SEQ ID NO: 2 - protein, 11 aa, synthetic construct
  REGION 1..11: note = Synthetic peptide
GGLGPKGQTGG

SEQ ID NO: 3 - protein, 10 aa, synthetic construct
  REGION 1..10: note = Synthetic peptide
GILSRIVGGG

SEQ ID NO: 4 - protein, 10 aa, synthetic construct
  REGION 1..10: note = Synthetic peptide
GGPQGIWGQK

SEQ ID NO: 5 - protein, 9 aa, synthetic construct
  REGION 1..9: note = Synthetic peptide
GGPVGLIGK

SEQ ID NO: 6 - protein, 11 aa, synthetic construct
  REGION 1..11: note = Synthetic peptide
GGPVPLSLVMK

SEQ ID NO: 7 - protein, 10 aa, synthetic construct
  REGION 1..10: note = Synthetic peptide
GGPLGLRSWK

SEQ ID NO: 8 - protein, 10 aa, synthetic construct
  REGION 1..10: note = Synthetic peptide
GGPLGVRGKK

SEQ ID NO: 9 - protein, 10 aa, synthetic construct
  REGION 1..10: note = Synthetic peptide
  SITE 3: note = MISC_FEATURE - D amino acid
GGFPRSGGGK

SEQ ID NO: 10 - protein, 11 aa, synthetic construct
  REGION 1..11: note = Synthetic peptide
GGLGPKGQTGK

SEQ ID NO: 11 - protein, 12 aa, synthetic construct
  REGION 1..12: note = Synthetic peptide
GGGSGRSANAKG

SEQ ID NO: 12 - protein, 10 aa, synthetic construct
  REGION 1..10: note = Synthetic peptide
GKPISLISSG

SEQ ID NO: 13 - protein, 10 aa, synthetic construct
  REGION 1..10: note = Synthetic peptide
GILSRIVGGG

SEQ ID NO: 14 - protein, 10 aa, synthetic construct
  REGION 1..10: note = Synthetic peptide
GSGSKIIGGG

SEQ ID NO: 15 - protein, 9 aa, synthetic construct
  REGION 1..9: note = Synthetic peptide
GGPLGMRGG

SEQ ID NO: 16 - protein, 9 aa, synthetic construct
  REGION 1..9: note = Synthetic peptide
GAPFEMSAG

SEQ ID NO: 17 - protein, 9 aa, synthetic construct
  REGION 1..9: note = Synthetic peptide
GRQRRVVGG

SEQ ID NO: 18 - protein, 9 aa, synthetic construct
  REGION 1..9: note = Synthetic peptide
GRQARAVGG

SEQ ID NO: 19 - protein, 9 aa, synthetic construct
  REGION 1..9: note = Synthetic peptide
GRRARVVGG

SEQ ID NO: 20 - protein, 9 aa, synthetic construct
  REGION 1..9: note = Synthetic peptide
GPLGLRSWG

SEQ ID NO: 21 - protein, 11 aa, synthetic construct
  REGION 1..11: note = Synthetic peptide
GWEAVRSAMWG

SEQ ID NO: 22 - protein, 11 aa, synthetic construct
  REGION 1..11: note = Synthetic peptide
GWIGFRNAGAG

SEQ ID NO: 23 - protein, 8 aa, synthetic construct
  REGION 1..8: note = Synthetic peptide
GPLGFRVG

SEQ ID NO: 24 - protein, 7 aa, synthetic construct
  REGION 1..7: note = Synthetic peptide
GPLGLRG

SEQ ID NO: 25 - protein, 11 aa, synthetic construct
  REGION 1..11: note = Synthetic peptide
GATPKIFNTEG

SEQ ID NO: 26 - protein, 11 aa, synthetic construct
  REGION 1..11: note = Synthetic peptide
GETRIIKGFEG

SEQ ID NO: 27 - protein, 9 aa, synthetic construct
  REGION 1..9: note = Synthetic peptide
GPLALWARG

SEQ ID NO: 28 - protein, 10 aa, synthetic construct
  REGION 1..10: note = Synthetic peptide
GALVPRGSAG

SEQ ID NO: 29 - protein, 9 aa, synthetic construct
  REGION 1..9: note = Synthetic peptide
GGLVPRGSG

SEQ ID NO: 30 - protein, 9 aa, synthetic construct
  REGION 1..9: note = Synthetic peptide
GRQARQVGG

SEQ ID NO: 31 - protein, 9 aa, synthetic construct
  REGION 1..9: note = Synthetic peptide
GLQARVVGG

SEQ ID NO: 32 - protein, 12 aa, synthetic construct
  REGION 1..12: note = Synthetic peptide
GGGSGRSANAKG

SEQ ID NO: 33 - protein, 7 aa, synthetic construct
  REGION 1..7: note = Synthetic peptide
GGVPRGG

SEQ ID NO: 34 - protein, 10 aa, synthetic construct
  REGION 1..10: note = Synthetic peptide
GRQSRIVGGG

SEQ ID NO: 35 - protein, 10 aa, synthetic construct
  REGION 1..10: note = Synthetic peptide
GILSRIVGGG

SEQ ID NO: 36 - protein, 11 aa, synthetic construct
  REGION 1..11: note = Synthetic peptide
GGRKRKVGGSG

SEQ ID NO: 37 - protein, 12 aa, synthetic construct
  REGION 1..12: note = Synthetic peptide
GDEKGKARDFFG

SEQ ID NO: 38 - protein, 10 aa, synthetic construct
  REGION 1..10: note = Synthetic peptide
GKPISLISSG

SEQ ID NO: 39 - protein, 9 aa, synthetic construct
  REGION 1..9: note = Synthetic peptide
GLAQAFRSG

SEQ ID NO: 40 - protein, 11 aa, synthetic construct
  REGION 1..11: note = Synthetic peptide
GRPLALWESQG

SEQ ID NO: 41 - protein, 13 aa, synthetic construct
  REGION 1..13: note = Synthetic peptide
  SITE 3: note = MISC_FEATURE - X is ornithine
GGXRSHPFTLYTA

SEQ ID NO: 42 - protein, 10 aa, synthetic construct
  REGION 1..10: note = Synthetic peptide
GRQRRALEKG

SEQ ID NO: 43 - protein, 10 aa, synthetic construct
  REGION 1..10: note = Synthetic peptide
GIQQRSLGGG

SEQ ID NO: 44 - protein, 13 aa, synthetic construct
  REGION 1..13: note = Synthetic peptide
GLRGGKGGPPPPG

SEQ ID NO: 45 - protein, 10 aa, synthetic construct
  REGION 1..10: note = Synthetic peptide
GGGAPFEMSA

SEQ ID NO: 46 - protein, 9 aa, synthetic construct
  REGION 1..9: note = Synthetic peptide
GPRPFNYLG

SEQ ID NO: 47 - protein, 10 aa, synthetic construct
  REGION 1..10: note = Synthetic peptide
GAGPRKAAKG

SEQ ID NO: 48 - protein, 10 aa, synthetic construct
  REGION 1..10: note = Synthetic peptide
GAKIRKAKKG

SEQ ID NO: 49 - protein, 10 aa, synthetic construct
  REGION 1..10: note = Synthetic peptide
GAKIRGQAKG

SEQ ID NO: 50 - protein, 10 aa, synthetic construct
  REGION 1..10: note = Synthetic peptide
GNSGRAVTYG

SEQ ID NO: 51 - protein, 10 aa, synthetic construct
  REGION 1..10: note = Synthetic peptide
GTYSRSRYLG

SEQ ID NO: 52 - protein, 10 aa, synthetic construct
  REGION 1..10: note = Synthetic peptide
GRPKPQQFWG

SEQ ID NO: 53 - protein, 12 aa, synthetic construct
  REGION 1..12: note = Synthetic peptide
GMAALIVRPDLG

SEQ ID NO: 54 - protein, 10 aa, synthetic construct
  REGION 1..10: note = Synthetic peptide
GGRIFLRTAG

SEQ ID NO: 55 - protein, 10 aa, synthetic construct
  REGION 1..10: note = Synthetic peptide
  SITE 8: note = MISC_FEATURE - D amino acid
  SITE 9: note = MISC_FEATURE - D amino acid
GSGDRMWGGG

SEQ ID NO: 56 - protein, 10 aa, synthetic construct
  REGION 1..10: note = Synthetic peptide
  SITE 9: note = MISC_FEATURE - D amino acid
GSGERMMGGG

SEQ ID NO: 57 - protein, 10 aa, synthetic construct
  REGION 1..10: note = Synthetic peptide
  SITE 2: note = MISC_FEATURE - D amino acid
  SITE 9: note = MISC_FEATURE - D amino acid
GSDDRRAGGG

SEQ ID NO: 58 - protein, 11 aa, synthetic construct
  REGION 1..11: note = Synthetic peptide
GKLRVVGGHPG

SEQ ID NO: 59 - protein, 12 aa, synthetic construct
  REGION 1..12: note = Synthetic peptide
GMAALITRPDFG

SEQ ID NO: 60 - protein, 12 aa, synthetic construct
  REGION 1..12: note = Synthetic peptide
GELIQRNLSPAG

SEQ ID NO: 61 - protein, 12 aa, synthetic construct
  REGION 1..12: note = Synthetic peptide
GSNLTRIVGGQG

SEQ ID NO: 62 - protein, 12 aa, synthetic construct
  REGION 1..12: note = Synthetic peptide
GYQIKPLKSTDG

SEQ ID NO: 63 - protein, 12 aa, synthetic construct
  REGION 1..12: note = Synthetic peptide
GNIPMGLLYNKG

SEQ ID NO: 64 - protein, 9 aa, synthetic construct
  REGION 1..9: note = Synthetic peptide
GPQGRIVGG

SEQ ID NO: 65 - protein, 9 aa, synthetic construct
  REGION 1..9: note = Synthetic peptide
GIKPRIVGG

SEQ ID NO: 66 - protein, 9 aa, synthetic construct
  REGION 1..9: note = Synthetic peptide
GPMKRLTLG

SEQ ID NO: 67 - protein, 10 aa, synthetic construct
  REGION 1..10: note = Synthetic peptide
GGLGPKGQTG

SEQ ID NO: 68 - protein, 10 aa, synthetic construct
  REGION 1..10: note = Synthetic peptide
GGAGGAQMGA

SEQ ID NO: 69 - protein, 10 aa, synthetic construct
  REGION 1..10: note = Synthetic peptide
GGATDVTTTP

SEQ ID NO: 70 - protein, 10 aa, synthetic construct
  REGION 1..10: note = Synthetic peptide
GGLSLPETGE

SEQ ID NO: 71 - protein, 12 aa, synthetic construct
  REGION 1..12: note = Synthetic peptide
GSPLAQAVRSSS

SEQ ID NO: 72 - protein, 14 aa, synthetic construct
  REGION 1..14: note = Synthetic peptide
GSSMAQTLTLRSSS

SEQ ID NO: 73 - protein, 10 aa, synthetic construct
  REGION 1..10: note = Synthetic peptide
GGGPLGLARG

SEQ ID NO: 74 - protein, 7 aa, synthetic construct
  REGION 1..7: note = Synthetic peptide
GMERMGG

SEQ ID NO: 75 - protein, 7 aa, synthetic construct
  REGION 1..7: note = Synthetic peptide
GGGGPGG

SEQ ID NO: 76 - protein, 9 aa, synthetic construct
  REGION 1..9: note = Synthetic peptide
GEWWMDYQG

SEQ ID NO: 77 - protein, 11 aa, synthetic construct
  REGION 1..11: note = Synthetic peptide
GFLRRQFKVVT

SEQ ID NO: 78 - protein, 11 aa, synthetic construct
  REGION 1..11: note = Synthetic peptide
GVGRPEWWMDG

SEQ ID NO: 79 - protein, 12 aa, synthetic construct
  REGION 1..12: note = Synthetic peptide
GLGALLRVKRLE

SEQ ID NO: 80 - protein, 10 aa, synthetic construct
  REGION 1..10: note = Synthetic peptide
GLASASTMDG

SEQ ID NO: 81 - protein, 10 aa, synthetic construct
  REGION 1..10: note = Synthetic peptide
GGKGRGLSLG

SEQ ID NO: 82 - protein, 10 aa, synthetic construct
  REGION 1..10: note = Synthetic peptide
GHGDQMAQKS

SEQ ID NO: 83 - protein, 10 aa, synthetic construct
  REGION 1..10: note = Synthetic peptide
GRAEQQRLKG

SEQ ID NO: 84 - protein, 9 aa, synthetic construct
  REGION 1..9: note = Synthetic peptide
GRAEQQRLG

SEQ ID NO: 85 - protein, 10 aa, synthetic construct
  REGION 1..10: note = Synthetic peptide
GDQMAQKSQG

SEQ ID NO: 86 - protein, 8 aa, synthetic construct
  REGION 1..8: note = Synthetic peptide
GAIEFDSG

SEQ ID NO: 87 - protein, 9 aa, synthetic construct
  REGION 1..9: note = Synthetic peptide
GGPQGIWGQ

SEQ ID NO: 88 - protein, 10 aa, synthetic construct
  REGION 1..10: note = Synthetic peptide
GGPVPLSLVM

SEQ ID NO: 89 - protein, 9 aa, synthetic construct
  REGION 1..9: note = Synthetic peptide
GGPLGVRGK

SEQ ID NO: 90 - protein, 9 aa, synthetic construct
  REGION 1..9: note = Synthetic peptide
  SITE 3: note = MISC_FEATURE - D amino acid
GGFPRSGGG

SEQ ID NO: 91 - protein, 10 aa, synthetic construct
  REGION 1..10: note = Synthetic peptide
GSGSKIIGGG

SEQ ID NO: 92 - protein, 7 aa, synthetic construct
  REGION 1..7: note = Synthetic peptide
GPLGMRG

SEQ ID NO: 93 - protein, 9 aa, synthetic construct
  REGION 1..9: note = Synthetic peptide
GAPFEMSAG

SEQ ID NO: 94 - protein, 10 aa, synthetic construct
  REGION 1..10: note = Synthetic peptide
  SITE 4: note = MISC_FEATURE - X is 3-cyclohexylalanine
  SITE 6: note = MISC_FEATURE - methyl-cysteine
GGPXGCHAGC

SEQ ID NO: 95 - protein, 8 aa, synthetic construct
  REGION 1..8: note = Synthetic peptide
GGPVGLIG

SEQ ID NO: 96 - protein, 10 aa, synthetic construct
  REGION 1..10: note = Synthetic peptide
GGAAEAISDA

SEQ ID NO: 97 - protein, 10 aa, synthetic construct
  REGION 1..10: note = Synthetic peptide
GGAQPDALNV

SEQ ID NO: 98 - protein, 10 aa, synthetic construct
  REGION 1..10: note = Synthetic peptide
GGDIVTVANA

SEQ ID NO: 99 - protein, 10 aa, synthetic construct
  REGION 1..10: note = Synthetic peptide
GGDLGLKSVP

SEQ ID NO: 100 - protein, 10 aa, synthetic construct
  REGION 1..10: note = Synthetic peptide
GGDVMASQKR

SEQ ID NO: 101 - protein, 10 aa, synthetic construct
  REGION 1..10: note = Synthetic peptide
GGESDELQTI

SEQ ID NO: 102 - protein, 10 aa, synthetic construct
  REGION 1..10: note = Synthetic peptide
GGFHPLHSKI

SEQ ID NO: 103 - protein, 10 aa, synthetic construct
  REGION 1..10: note = Synthetic peptide
GGGHARLVHV

SEQ ID NO: 104 - protein, 10 aa, synthetic construct
  REGION 1..10: note = Synthetic peptide
GGHIANVERV

SEQ ID NO: 105 - protein, 10 aa, synthetic construct
  REGION 1..10: note = Synthetic peptide
GGKAAATQKK

SEQ ID NO: 106 - protein, 10 aa, synthetic construct
  REGION 1..10: note = Synthetic peptide
GGLATASTMD

SEQ ID NO: 107 - protein, 10 aa, synthetic construct
  REGION 1..10: note = Synthetic peptide
GGNLAGILKE

SEQ ID NO: 108 - protein, 10 aa, synthetic construct
  REGION 1..10: note = Synthetic peptide
GGNPGMSEPV

SEQ ID NO: 109 - protein, 9 aa, synthetic construct
  REGION 1..9: note = Synthetic peptide
GGPFGCHAK

SEQ ID NO: 110 - protein, 9 aa, synthetic construct
  REGION 1..9: note = Synthetic peptide
GGPLGLRWW

SEQ ID NO: 111 - protein, 10 aa, synthetic construct
  REGION 1..10: note = Synthetic peptide
GGQMGVMQGV

SEQ ID NO: 112 - protein, 10 aa, synthetic construct
  REGION 1..10: note = Synthetic peptide
GGQTCKCSCK

SEQ ID NO: 113 - protein, 10 aa, synthetic construct
  REGION 1..10: note = Synthetic peptide
GGQWAGLVEK

SEQ ID NO: 114 - protein, 10 aa, synthetic construct
  REGION 1..10: note = Synthetic peptide
GGRPAVMTSP

SEQ ID NO: 115 - protein, 10 aa, synthetic construct
  REGION 1..10: note = Synthetic peptide
GGTLRELHLD

SEQ ID NO: 116 - protein, 10 aa, synthetic construct
  REGION 1..10: note = Synthetic peptide
GGTPPPSQGK

SEQ ID NO: 117 - protein, 10 aa, synthetic construct
  REGION 1..10: note = Synthetic peptide
GGTSEDLVVQ

SEQ ID NO: 118 - protein, 10 aa, synthetic construct
  REGION 1..10: note = Synthetic peptide
GGVWAAEAIS

SEQ ID NO: 119 - protein, 12 aa, synthetic construct
  REGION 1..12: note = Synthetic peptide
  SITE 8: note = MISC_FEATURE - X is norvaline
GRPKPVEXWRKG

SEQ ID NO: 120 - protein, 8 aa, synthetic construct
  REGION 1..8: note = Synthetic peptide
GHSSKLQG

SEQ ID NO: 121 - protein, 10 aa, synthetic construct
  REGION 1..10: note = Synthetic peptide
GSSQYSSNGG

SEQ ID NO: 122 - protein, 10 aa, synthetic construct
  REGION 1..10: note = Synthetic peptide
GQKGRYKQEG

SEQ ID NO: 123 - protein, 10 aa, synthetic construct
  REGION 1..10: note = Synthetic peptide
GGKAFRRSGG

SEQ ID NO: 124 - protein, 8 aa, synthetic construct
  REGION 1..8: note = Synthetic peptide
GAANLTRG

SEQ ID NO: 125 - protein, 7 aa, synthetic construct
  REGION 1..7: note = Synthetic peptide
GGGELRG

SEQ ID NO: 126 - protein, 9 aa, synthetic construct
  REGION 1..9: note = Synthetic peptide
  SITE 6: note = MISC_FEATURE - X is homo-phenylalanine
GLAQAXRSG

SEQ ID NO: 127 - protein, 12 aa, synthetic construct
  REGION 1..12: note = Synthetic peptide
GSPLAQAVRSSG

SEQ ID NO: 128 - protein, 10 aa, synthetic construct
  REGION 1..10: note = Synthetic peptide
GPVPLSLVMG

SEQ ID NO: 129 - protein, 10 aa, synthetic construct
  REGION 1..10: note = Synthetic peptide
GSQPRIVGGG

SEQ ID NO: 130 - protein, 8 aa, synthetic construct
  REGION 1..8: note = Synthetic peptide
GSYRIFGG

SEQ ID NO: 131 - protein, 8 aa, synthetic construct
  REGION 1..8: note = Synthetic peptide
RPKPVEGG

SEQ ID NO: 132 - protein, 14 aa, synthetic construct
  REGION 1..14: note = Synthetic peptide
  SITE 10: note = MISC_FEATURE - O-methyl benzyl
GRRRGGAANCRMGG

SEQ ID NO: 133 - protein, 10 aa, synthetic construct
  REGION 1..10: note = Synthetic peptide
GVPLSLTMGG

SEQ ID NO: 134 - protein, 9 aa, synthetic construct
  REGION 1..9: note = Synthetic peptide
GAPFEFSAG

SEQ ID NO: 135 - protein, 10 aa, synthetic construct
  REGION 1..10: note = Synthetic peptide
GRPLALWRSG

SEQ ID NO: 136 - protein, 11 aa, synthetic construct
  REGION 1..11: note = Synthetic peptide
GRPLALEESQG

SEQ ID NO: 137 - protein, 11 aa, synthetic construct
  REGION 1..11: note = Synthetic peptide
GRPLALWRSQG

SEQ ID NO: 138 - protein, 13 aa, synthetic construct
  REGION 1..13: note = Synthetic peptide
GRNALAVERTASG

SEQ ID NO: 139 - protein, 11 aa, synthetic construct
  REGION 1..11: note = Synthetic peptide
  SITE 8: note = MISC_FEATURE - X is norvaline
GRPKPVEXWRG

SEQ ID NO: 140 - protein, 12 aa, synthetic construct
  REGION 1..12: note = Synthetic peptide
GSGSNPYKYTAG

SEQ ID NO: 141 - protein, 12 aa, synthetic construct
  REGION 1..12: note = Synthetic peptide
GSGSNPYGYTAG

SEQ ID NO: 142 - protein, 12 aa, synthetic construct
  REGION 1..12: note = Synthetic peptide
GSGTLSELHTAG

SEQ ID NO: 143 - protein, 12 aa, synthetic construct
  REGION 1..12: note = Synthetic peptide
GSGTISHLHTAG

SEQ ID NO: 144 - protein, 15 aa, synthetic construct
  REGION 1..15: note = Synthetic peptide
  SITE 4: note = X is ornithine
  SITE 9: note = homo-phenylalanine
GSGXRSHPFTLYTAG

SEQ ID NO: 145 - protein, 15 aa, synthetic construct
  REGION 1..15: note = Synthetic peptide
  SITE 4: note = MISC_FEATURE - X is ornithine
  SITE 9: note = MISC_FEATURE - homo-phenylalanine
GSGXRSHGFFLYTAG

SEQ ID NO: 146 - protein, 12 aa, synthetic construct
  REGION 1..12: note = Synthetic peptide
GSGESLAYYTAG

SEQ ID NO: 147 - protein, 12 aa, synthetic construct
  REGION 1..12: note = Synthetic peptide
GSGHMHAALTAG

SEQ ID NO: 148 - protein, 12 aa, synthetic construct
  REGION 1..12: note = Synthetic peptide
GSGKGPRQITAG

SEQ ID NO: 149 - protein, 12 aa, synthetic construct
  REGION 1..12: note = Synthetic peptide
GSGPLFYSVTAG

SEQ ID NO: 150 - protein, 12 aa, synthetic construct
  REGION 1..12: note = Synthetic peptide
GSGRSENIRTAG

SEQ ID NO: 151 - protein, 10 aa, synthetic construct
  REGION 1..10: note = Synthetic peptide
GGPWGIWGQG

SEQ ID NO: 152 - protein, 9 aa, synthetic construct
  REGION 1..9: note = Synthetic peptide
  SITE 3: note = MISC_FEATURE - D amino acid
  SITE 4: note = MISC_FEATURE - X is pipecolinic acid
GGFXRSGGG

SEQ ID NO: 153 - protein, 9 aa, synthetic construct
  REGION 1..9: note = Synthetic peptide
  SITE 3: note = MISC_FEATURE - D amino acid
  SITE 4: note = MISC_FEATURE - X is pipecolinic acid
GGFXKSGGG

SEQ ID NO: 154 - protein, 9 aa, synthetic construct
  REGION 1..9: note = Synthetic peptide
GGHPGGPQG

SEQ ID NO: 155 - protein, 11 aa, synthetic construct
  REGION 1..11: note = Synthetic peptide
GGGVFRMLSVG

SEQ ID NO: 156 - protein, 11 aa, synthetic construct
  REGION 1..11: note = Synthetic peptide
GGGLFRSLSSG

SEQ ID NO: 157 - protein, 10 aa, synthetic construct
  REGION 1..10: note = Synthetic peptide
GGGLLYGKGG

SEQ ID NO: 158 - protein, 7 aa, synthetic construct
  REGION 1..7: note = Synthetic peptide
  SITE 2: note = MISC_FEATURE - D amino acid
  SITE 3: note = MISC_FEATURE - D amino acid
  SITE 4: note = MISC_FEATURE - X is 1,2,3,4-tetrahydroisoquinoline-3-carboxylic acid
GGYXTNG

SEQ ID NO: 159 - protein, 12 aa, synthetic construct
  REGION 1..12: note = Synthetic peptide
GGIPSIQSRGLG

SEQ ID NO: 160 - protein, 13 aa, synthetic construct
  REGION 1..13: note = Synthetic peptide
GGNLARALKQTIG

SEQ ID NO: 161 - protein, 13 aa, synthetic construct
  REGION 1..13: note = Synthetic peptide
GGHMVQHLIQWHG

SEQ ID NO: 162 - protein, 12 aa, synthetic construct
  REGION 1..12: note = Synthetic peptide
  SITE 8: note = MISC_FEATURE - homo-phenylalanine
GGPRAAAFTSPG

SEQ ID NO: 163 - protein, 10 aa, synthetic construct
  REGION 1..10: note = Synthetic peptide
GGTGPPGYTG

SEQ ID NO: 164 - protein, 10 aa, synthetic construct
  REGION 1..10: note = Synthetic peptide
GGTGLPVYQG

SEQ ID NO: 165 - skipped

SEQ ID NO: 166 - protein, 7 aa, synthetic construct
  REGION 1..7: note = Synthetic peptide
GGAAFAG

SEQ ID NO: 167 - protein, 31 aa, synthetic construct
  REGION 1..31: note = Synthetic peptide
  SITE 1: note = MISC_FEATURE - N-terminal QSY21 fluorophore
  REGION 10..11: note = MISC_FEATURE - (poly)ethylene-glycol, MW 2000 g/mol between residue 10 and 11
  SITE 11: note = MISC_FEATURE - X is 5-amino-3-oxopentanoyl, or another residue for physicaldistancing and/or stabilization, or is absent
  SITE 31: note = MISC_FEATURE - C-terminal CY5 fluorophore
  NON_STD 1..9: note = D amino acid
  NON_STD 22..30: note = D amino acid
EEEEEEEEECXGGPQGIWGQGRRRRRRRRRK

SEQ ID NO: 168 - protein, 31 aa, synthetic construct
  REGION 1..31: note = Synthetic peptide
  SITE 1: note = MISC_FEATURE - X is is succinoyl or is absent
  SITE 10: note = MISC_FEATURE - X is 6-aminohexanoyl or is absent
  SITE 30: note = MISC_FEATURE - X is 6-aminohexanoyl or is absent
  SITE 31: note = MISC_FEATURE - C-terminal Cy5 fluorophore
  NON_STD 2..9: note = D amino acid
  NON_STD 21..29: note = D amino acid
  NON_STD 31: note = D amino acid
XEEEEEEEEXGGPQGIWGQGRRRRRRRRRXK

SEQ ID NO: 169 - protein, 32 aa, synthetic construct
  REGION 1..32: note = Synthetic peptide
  SITE 1: note = MISC_FEATURE - X is is succinoyl or is absent
  SITE 10: note = MISC_FEATURE - X is 6-aminohexanoyl or is absent
  SITE 31: note = MISC_FEATURE - X is 6-aminohexanoyl or is absent
  SITE 32: note = MISC_FEATURE - C-terminal Cy3 fluorophore
  NON_STD 2..9: note = D amino acid
  NON_STD 22..30: note = D amino acid
  NON_STD 32: note = D amino acid
XEEEEEEEEXGGLGPKGQTGGRRRRRRRRRXK

SEQ ID NO: 170 - protein, 31 aa, synthetic construct
  REGION 1..31: note = Synthetic peptide
  SITE 1: note = MISC_FEATURE - X is is succinoyl or is absent
  SITE 10: note = MISC_FEATURE - X is 6-aminohexanoyl or is absent
  SITE 30: note = MISC_FEATURE - X is 6-aminohexanoyl or is absent
  SITE 31: note = MISC_FEATURE - C-terminal FAM fluorophore
  NON_STD 2..9: note = D amino acid
  NON_STD 21..29: note = D amino acid
  NON_STD 31: note = D amino acid
XEEEEEEEEXGILSRIVGGGRRRRRRRRRXK

SEQ ID NO: 171 - protein, 31 aa, synthetic construct; identical to SEQ ID NO: 168
  REGION 1..31: note = Synthetic peptide
  SITE 1: note = misc_feature - Xaa can be succinoyl or is absent
  SITE 10: note = misc_feature - Xaa can be 6-aminohexanoyl or is absent
  SITE 30: note = misc_feature - Xaa can be 6-aminohexanoyl or is absent
  NON_STD 2..9: note = D amino acid
  NON_STD 21..29: note = D amino acid
  MOD_RES 31: note = C terminal Cy3

SEQ ID NO: 172 - protein, 31 aa, synthetic construct
  REGION 1..31: note = Synthetic peptide
  SITE 1: note = misc_feature - Xaa can be succinoyl or is absent
  SITE 10: note = misc_feature - Xaa can be 6-aminohexanoyl or is absent
  SITE 30: note = misc_feature - Xaa can be 6-aminohexanoyl or is absent
  NON_STD 2..9: note = D amino acid
  NON_STD 21..29: note = D amino acid
  MOD_RES 31: note = C terminal Cy5
XEEEEEEEEXGGLVPRGSGGRRRRRRRRRXK

SEQ ID NO: 173 - protein, 30 aa, synthetic construct
  REGION 1..30: note = Synthetic peptide
  SITE 1: note = misc_feature - Xaa can be succinoyl or is absent
  SITE 10: note = misc_feature - Xaa can be 6-aminohexanoyl or is absent
  SITE 29: note = misc_feature - Xaa can be 6-aminohexanoyl or is absent
  NON_STD 2..9: note = D amino acid
  NON_STD 20..28: note = D amino acid
  MOD_RES 30: note = C terminal 5FAM
XEEEEEEEEXGGPVGLIGGRRRRRRRRRXK

SEQ ID NO: 174 - protein, 30 aa, synthetic construct
  REGION 1..30: note = Synthetic peptide
  SITE 1: note = misc_feature - Xaa can be succinoyl or is absent
  SITE 10: note = misc_feature - Xaa can be 6-aminohexanoyl or is absent
  SITE 29: note = misc_feature - Xaa can be 6-aminohexanoyl or is absent
  NON_STD 2..9: note = D amino acid
  NON_STD 20..28: note = D amino acid
  MOD_RES 30: note = C terminal 5FAM
XEEEEEEEEXGPLGVRGKGRRRRRRRRRXK

SEQ ID NO: 175 - protein, 31 aa, synthetic construct
  REGION 1..31: note = Synthetic peptide
  SITE 1: note = misc_feature - Xaa can be succinoyl or is absent
  SITE 10: note = misc_feature - Xaa can be 6-aminohexanoyl or is absent
  SITE 30: note = misc_feature - Xaa can be 6-aminohexanoyl or is absent
  NON_STD 2..9: note = D amino acid
  NON_STD 21..29: note = D amino acid
  MOD_RES 31: note = C terminal Cy3
XEEEEEEEEXGRQRRALEKGRRRRRRRRRXK

SEQ ID NO: 176 - protein, 30 aa, synthetic construct
  REGION 1..30: note = Synthetic peptide
  SITE 1: note = misc_feature - Xaa can be succinoyl or is absent
  SITE 10: note = misc_feature - Xaa can be 6-aminohexanoyl or is absent
  SITE 29: note = misc_feature - Xaa can be 6-aminohexanoyl or is absent
  NON_STD 2..9: note = D amino acid
  NON_STD 20..28: note = D amino acid
  MOD_RES 30: note = C terminal Cy5
XEEEEEEEEXGSGRSANAGRRRRRRRRRXK

SEQ ID NO: 177 - protein, 31 aa, synthetic construct
  REGION 1..31: note = Synthetic peptide
  SITE 1: note = misc_feature - Xaa can be succinoyl or is absent
  SITE 10: note = misc_feature - Xaa can be 6-aminohexanoyl or is absent
  SITE 30: note = misc_feature - Xaa can be 6-aminohexanoyl or is absent
  NON_STD 2..9: note = D amino acid
  NON_STD 21..29: note = D amino acid
  MOD_RES 31: note = C terminal 5FAM
XEEEEEEEEXGKPISLISSGRRRRRRRRRXK

SEQ ID NO: 178 - protein, 31 aa, synthetic construct; identical to SEQ ID NO: 170
  REGION 1..31: note = Synthetic peptide
  SITE 1: note = misc_feature - Xaa can be succinoyl or is absent
  SITE 10: note = misc_feature - Xaa can be 6-aminohexanoyl or is absent
  SITE 30: note = misc_feature - Xaa can be 6-aminohexanoyl or is absent
  NON_STD 2..9: note = D amino acid
  NON_STD 21..29: note = D amino acid
  MOD_RES 31: note = C terminal 5FAM

SEQ ID NO: 179 - protein, 33 aa, synthetic construct
  REGION 1..33: note = Synthetic peptide
  SITE 1: note = misc_feature - Xaa can be succinoyl or is absent
  SITE 10: note = misc_feature - Xaa can be 6-aminohexanoyl or is absent
  SITE 32: note = misc_feature - Xaa can be 6-aminohexanoyl or is absent
  NON_STD 2..9: note = D amino acid
  NON_STD 18: note = X is norvaline
  MOD_RES 33: note = C terminal 5FAM
  NON_STD 23..30: note = D amino acid
XEEEEEEEEXGRPKPVEXWRKGRRRRRRRRRXK

SEQ ID NO: 180 - protein, 31 aa, synthetic construct
  REGION 1..31: note = Synthetic peptide
  SITE 1: note = misc_feature - Xaa can be succinoyl or is absent
  SITE 10: note = misc_feature - Xaa can be 6-aminohexanoyl or is absent
  SITE 30: note = misc_feature - Xaa can be 6-aminohexanoyl or is absent
  NON_STD 2..9: note = D amino acid
  NON_STD 21..29: note = D amino acid
  MOD_RES 31: note = C terminal 5FAM
XEEEEEEEEXGIQQRSLGGGRRRRRRRRRXK

SEQ ID NO: 181 - protein, 30 aa, synthetic construct
  REGION 1..30: note = Synthetic peptide
  SITE 1: note = misc_feature - Xaa can be succinoyl or is absent
  SITE 10: note = misc_feature - Xaa can be 6-aminohexanoyl or is absent
  SITE 29: note = misc_feature - Xaa can be 6-aminohexanoyl or is absent
  NON_STD 2..9: note = D amino acid
  NON_STD 20..28: note = D amino acid
  MOD_RES 30: note = C terminal 5FAM
XEEEEEEEEXGGGVPRGGGRRRRRRRRRXK

SEQ ID NO: 182 - protein, 31 aa, synthetic construct
  REGION 1..31: note = Synthetic peptide
  SITE 1: note = misc_feature - Xaa can be succinoyl or is absent
  SITE 10: note = misc_feature - Xaa can be 6-aminohexanoyl or is absent
  SITE 30: note = misc_feature - Xaa can be 6-aminohexanoyl or is absent
  NON_STD 2..9: note = D amino acid
  NON_STD 21..29: note = D amino acid
  MOD_RES 31: note = C terminal Cy3
XEEEEEEEEXGSGSKIIGGGRRRRRRRRRXK

SEQ ID NO: 183 - protein, 31 aa, synthetic construct
  REGION 1..31: note = Synthetic peptide
  SITE 1: note = misc_feature - Xaa can be succinoyl or is absent
  SITE 10: note = misc_feature - Xaa can be 6-aminohexanoyl or is absent
  SITE 30: note = misc_feature - Xaa can be 6-aminohexanoyl or is absent
  NON_STD 2..9: note = D amino acid
  NON_STD 21..29: note = D amino acid
  MOD_RES 31: note = C terminal Cy3
XEEEEEEEEXGGAANLTRGGRRRRRRRRRXK

SEQ ID NO: 184 - protein, 30 aa, synthetic construct
  REGION 1..30: note = Synthetic peptide
  SITE 1: note = misc_feature - Xaa can be succinoyl or is absent
  SITE 10: note = misc_feature - Xaa can be 6-aminohexanoyl or is absent
  SITE 29: note = misc_feature - Xaa can be 6-aminohexanoyl or is absent
  NON_STD 2..9: note = D amino acid
  NON_STD 20..28: note = D amino acid
  MOD_RES 30: note = C terminal Cy3
  NON_STD 26: note = homo-phenylalanine
XEEEEEEEEXGLAQAFRSGRRRRRRRRRXK

SEQ ID NO: 185 - protein, 33 aa, synthetic construct
  REGION 1..33: note = Synthetic peptide
  SITE 1: note = misc_feature - Xaa can be succinoyl or is absent
  SITE 10: note = misc_feature - Xaa can be 6-aminohexanoyl or is absent
  SITE 32: note = misc_feature - Xaa can be 6-aminohexanoyl or is absent
  NON_STD 2..9: note = D amino acid
  NON_STD 22..31: note = D amino acid
  MOD_RES 33: note = C terminal Cy3
XEEEEEEEEXGSPLAQAVRSSGRRRRRRRRRXK

SEQ ID NO: 186 - protein, 31 aa, synthetic construct
  REGION 1..31: note = Synthetic peptide
  SITE 1: note = misc_feature - Xaa can be succinoyl or is absent
  SITE 10: note = misc_feature - Xaa can be 6-aminohexanoyl or is absent
  SITE 30: note = misc_feature - Xaa can be 6-aminohexanoyl or is absent
  NON_STD 2..9: note = D amino acid
  NON_STD 21..29: note = D amino acid
  MOD_RES 31: note = C terminal 5FAM
XEEEEEEEEXGPVPLSLVMGRRRRRRRRRXK

SEQ ID NO: 187 - protein, 31 aa, synthetic construct
  REGION 1..31: note = Synthetic peptide
  SITE 1: note = misc_feature - Xaa can be succinoyl or is absent
  SITE 10: note = misc_feature - Xaa can be 6-aminohexanoyl or is absent
  SITE 30: note = misc_feature - Xaa can be 6-aminohexanoyl or is absent
  NON_STD 2..9: note = D amino acid
  NON_STD 21..29: note = D amino acid
  MOD_RES 31: note = C terminal Cy5
XEEEEEEEEXGSQPRIVGGGRRRRRRRRRXK

SEQ ID NO: 188 - protein, 32 aa, synthetic construct
  REGION 1..32: note = Synthetic peptide
  SITE 1: note = misc_feature - Xaa can be succinoyl or is absent
  SITE 10: note = misc_feature - Xaa can be 6-aminohexanoyl or is absent
  SITE 31: note = misc_feature - Xaa can be 6-aminohexanoyl or is absent
  NON_STD 2..9: note = D amino acid
  NON_STD 21..30: note = D amino acid
  MOD_RES 32: note = C terminal Cy3
XEEEEEEEEXGGGHARLVHVGRRRRRRRRRXK

SEQ ID NO: 189 - protein, 32 aa, synthetic construct; identical to SEQ ID NO: 169
  REGION 1..32: note = Synthetic peptide
  SITE 1: note = misc_feature - Xaa can be succinoyl or is absent
  SITE 10: note = misc_feature - Xaa can be 6-aminohexanoyl or is absent
  SITE 31: note = misc_feature - Xaa can be 6-aminohexanoyl or is absent
  NON_STD 2..9: note = D amino acid
  NON_STD 22..30: note = D amino acid
  MOD_RES 32: note = C terminal Cy3

SEQ ID NO: 190 - protein, 32 aa, synthetic construct
  REGION 1..32: note = Synthetic peptide
  SITE 1: note = misc_feature - Xaa can be succinoyl or is absent
  SITE 10: note = misc_feature - Xaa can be 6-aminohexanoyl or is absent
  SITE 31: note = misc_feature - Xaa can be 6-aminohexanoyl or is absent
  NON_STD 2..9: note = D amino acid
  NON_STD 22..30: note = D amino acid
  MOD_RES 32: note = C terminal Cy5
XEEEEEEEEXGGQTCKCSCKGRRRRRRRRRXK

SEQ ID NO: 191 - protein, 30 aa, synthetic construct; identical to SEQ ID NO: 176
  REGION 1..30: note = Synthetic peptide
  SITE 1: note = misc_feature - Xaa can be succinoyl or is absent
  SITE 10: note = misc_feature - Xaa can be 6-aminohexanoyl or is absent
  SITE 29: note = misc_feature - Xaa can be 6-aminohexanoyl or is absent
  NON_STD 2..9: note = D amino acid
  NON_STD 12: note = D amino acid
  NON_STD 14..18: note = D amino acid
  NON_STD 20..28: note = D amino acid
  MOD_RES 30: note = C terminal Cy5

SEQ ID NO: 192 - protein, 30 aa, synthetic construct
  REGION 1..30: note = Synthetic peptide
  SITE 11: note = misc_feature - Xaa can be 5-amino-3-oxopentanoyl or another residue for physical distancing and/or stabilization, or is absent
  MOD_RES 1: note = N terminal QSY21
  NON_STD 1..10: note = D amino acid
  NON_STD 21..29: note = D amino acid
  MOD_RES 30: note = C terminal Cy5
EEEEEEEEECXGSGRSANAGRRRRRRRRRK

SEQ ID NO: 193 - protein, 30 aa, synthetic construct; identical to SEQ ID NO: 192
  REGION 1..30: note = Synthetic peptide
  SITE 11: note = misc_feature - Xaa can be 5-amino-3-oxopentanoyl or another residue for physical distancing and/or stabilization, or is absent
  MOD_RES 1: note = N terminal QSY21
  NON_STD 1..10: note = D amino acid
  NON_STD 21..29: note = D amino acid
  MOD_RES 30: note = C terminal Cy5

SEQ ID NO: 194 - protein, 62 aa, synthetic construct
  REGION 1..62: note = Synthetic peptide
  SITE 1: note = misc_feature - Xaa can be succinoyl or is absent
  SITE 10: note = misc_feature - Xaa can be 6-aminohexanoyl or is absent
  SITE 15: note = misc_feature - Xaa can be succinoyl or is absent
  SITE 24: note = misc_feature - Xaa can be 6-aminohexanoyl or is absent
  SITE 44: note = misc_feature - Xaa can be 6-aminohexanoyl or is absent
  SITE 61: note = misc_feature - Xaa can be 6-aminohexanoyl or is absent
  NON_STD 2..9: note = D amino acid
  NON_STD 12..14: note = D amino acid
  NON_STD 16..23: note = D amino acid
  NON_STD 35..43: note = D amino acid
  NON_STD 46..50: note = D amino acid
  NON_STD 52..60: note = D amino acid
  MOD_RES 62: note = C terminal 5FAM
XEEEEEEEEXGPVPXEEEEEEEEXGRQRRALEKGRRRRRRRRRXKLSLVMGRRRRRRRRRXK

SEQ ID NO: 195 - protein, 31 aa, synthetic construct
  REGION 1..31: note = Synthetic peptide
  SITE 11: note = misc_feature - Xaa can be 5-amino-3-oxopentanoyl or another residue for physical distancing and/or stabilization, or is absent
  MOD_RES 1: note = N terminal QSY21
  NON_STD 1..10: note = D amino acid
  NON_STD 22..30: note = D amino acid
  MOD_RES 31: note = C terminal Cy5
EEEEEEEEECXGPVPLSLVMGRRRRRRRRRK

SEQ ID NO: 196 - protein, 31 aa, synthetic construct; identical to SEQ ID NO: 195
  REGION 1..31: note = Synthetic peptide
  SITE 11: note = misc_feature - Xaa can be 5-amino-3-oxopentanoyl or another residue for physical distancing and/or stabilization, or is absent
  MOD_RES 1: note = N terminal QSY21
  NON_STD 1..10: note = D amino acid
  NON_STD 13..20: note = D amino acid
  NON_STD 22..30: note = D amino acid
  MOD_RES 31: note = C terminal Cy5

SEQ ID NO: 197 - protein, 14 aa, synthetic construct [sequence omitted]

SEQ ID NO: 198 - protein, 14 aa, synthetic construct [sequence omitted]

SEQ ID NO: 199 - protein, 14 aa, synthetic construct [sequence omitted]

SEQ ID NO: 200 - protein, 14 aa, synthetic construct [sequence omitted]

SEQ ID NO: 201 - protein, 14 aa, synthetic construct [sequence omitted]

SEQ ID NO: 202 - protein, 14 aa, synthetic construct [sequence omitted]

SEQ ID NO: 203 - protein, 14 aa, synthetic construct [sequence omitted]

SEQ ID NO: 204 - protein, 14 aa, synthetic construct [sequence omitted]

SEQ ID NO: 205 - protein, 14 aa, synthetic construct [sequence omitted]

SEQ ID NO: 206 - protein, 14 aa, synthetic construct [sequence omitted]

SEQ ID NO: 207 - protein, 14 aa, synthetic construct [sequence omitted]

SEQ ID NO: 208 - protein, 14 aa, synthetic construct [sequence omitted]

SEQ ID NO: 209 - protein, 14 aa, synthetic construct [sequence omitted]

SEQ ID NO: 210 - protein, 14 aa, synthetic construct [sequence omitted]

SEQ ID NO: 211 - protein, 10 aa, synthetic construct
GGPQGIWGQC

SEQ ID NO: 212 - protein, 9 aa, synthetic construct
GGPVGLIGC

SEQ ID NO: 213 - protein, 10 aa, synthetic construct
GGPVPLSLVM

SEQ ID NO: 214 - protein, 10 aa, synthetic construct
GGPLGLRSWC

SEQ ID NO: 215 - protein, 10 aa, synthetic construct
GGPLGVRGKC

SEQ ID NO: 216 - protein, 10 aa, synthetic construct
  NON_STD 3: note = D amino acid
GGFPRSGGGC

SEQ ID NO: 217 - protein, 11 aa, synthetic construct
GGLGPKGQTGC

SEQ ID NO: 218 - protein, 13 aa, synthetic construct
GGGSGRSANAKGC

SEQ ID NO: 219 - protein, 12 aa, synthetic construct
GGKPISLISSGC

SEQ ID NO: 220 - protein, 12 aa, synthetic construct
GGILSRIVGGGC

SEQ ID NO: 221 - protein, 12 aa, synthetic construct
GGSGSKIIGGGC

SEQ ID NO: 222 - protein, 10 aa, synthetic construct
GGPLGMRGGC

SEQ ID NO: 223 - protein, 10 aa, synthetic construct
  NON_STD 4: note = X is 3-Cyclohexylalanine
  MOD_RES 6: note = methyl-cysteine
GGPXGCHAGC

SEQ ID NO: 224 - protein, 11 aa, synthetic construct
GGAPFEMSAGC

SEQ ID NO: 225 - protein, 11 aa, synthetic construct
  NON_STD 1..9: note = D amino acid
  NON_STD 10: note = X is 6-aminohexanoyl
  NON_STD 11: note = D amino acid
RRRRRRRRRXK

SEQ ID NO: 226 - protein, 10 aa, synthetic construct
GGPQGIWGQC

SEQ ID NO: 227 - protein, 10 aa, synthetic construct
GIQQRSLGGG

SEQ ID NO: 228 - protein, 9 aa, synthetic construct
GGLVPRGSG

(19) United States
(12) Patent Application Publication
(10) Pub. No.: US 2025/0003968 A1
(43) Pub. Date: Jan. 2, 2025

(54) ACTIVITY-BASED CELL SORTING

(71) Applicants: MASSACHUSETTS INSTITUTE OF TECHNOLOGY, Cambridge, MA (US); PRESIDENT AND FELLOWS OF HARVARD COLLEGE, Cambridge, MA (US)

(72) Inventors: Sangeeta BHATIA, Lexington, MA (US); Jesse KIRKPATRICK, Cambridge, MA (US); Ava SOLEIMANY, Newport Coast, CA (US)

(21) Appl. No.: 18/708,719

(22) PCT Filed: Nov. 9, 2022
(86) PCT No.: PCT/US2022/079553

§ 371 Date: May 9, 2024

Related U.S. Application Data
(60) Provisional application No. 63/278,234, filed on Nov. 11, 2021.

(51) Int. Cl. G01N 33/573; G01N 33/542

(52) U.S. Cl. CPC G01N 33/573; G01N 33/542; G01N 2333/96425

(57) ABSTRACT

Hie disclosure provides zymography probes of the general formula X1-X2-X3, wherein one of XI and X3 is a cationic peptide linked to a fluorophore, and the other is an anionic peptide, and X2 is protease-cleavable peptide, and their use for activity based cell sorting.

### CROSS REFERENCE

This application claims priority to U.S. Provisional Patent Application Ser. No. 63/278,234 filed Nov. 11, 2021, incorporated by reference herein in its entirety.

### SEQUENCE LISTING STATEMENT

A computer readable form of the Sequence Listing is filed with this application by electronic submission and is incorporated into this application by reference in its entirety. The sequence listing submitted herewith is contained in the file created Nov. 9, 2022, entitled “21-1392-WO_Sequence-Listing.xml” and is 398 kilobytes in size.

### BACKGROUND

Protease activity is dysregulated across multiple disease states, including cancer, fibrosis, and infection. As a result of this dysregulation, proteases are considered a potential diagnostic and therapeutic target of diseases including cancer. However, there is currently a dearth of methods to dissect protease activity in human disease.

### SUMMARY

In one aspect, the disclosure provides methods for activity based cell sorting, comprising
(a) contacting a biological sample with an activatable zymography probe (AZP), wherein the AZP comprises the general formula X1-X2-X3, wherein
   (i) one of X1 and X3 is a cationic peptide linked to a fluorophore, and the other is an anionic peptide; and
   (ii) X2 is protease-cleavable peptide,
wherein degradation of the protease-cleavable peptide by proteases present in the biological sample liberates the fluorophore-linked cationic peptide so that it binds cells at or near a site of protease degradation of the protease-cleavable peptide to produce fluorophore-tagged cells, and
(b) isolating the fluorophore tagged cells from the biological sample by fluorescence activated cell sorting (FACS).

In various embodiments, the cationic peptide only includes R, K, and/or H residues; and/or the anionic peptide only includes D and/or E residues. In one embodiment, the cationic peptide is a polyR peptide. In another embodiment, the anionic peptide is a polyE peptide. In various embodiments, the cationic domain and/or the anionic domain may include D amino acids, or may be exclusively D amino acids. In some embodiments, the fluorophore is selected from the group consisting of fluorescein phosphoramidides, rhodamine, polymethadine dye derivative, phosphores, Texas red, green fluorescent protein, Cy3, Cy5, and Cy7. In other embodiments, the fluorophore is a fluorophore component of a fluorophore-quencher pair, and the anionic peptide linked to the quencher component of the fluorophore-quencher pair. In other embodiments, the protease-cleavable peptide comprises the sequence selected from the group consisting of SEQ ID NO:1-196. In one embodiment, the AZP comprises the sequence selected from the group consisting of SEQ ID NO:167-196.

In another aspect, the disclosure provides probes, comprising the general formula X1-X2-X3, wherein
one of X1 and X3 is a cationic peptide linked to a detectable marker and the other is a anionic peptide;
X2 is a protease-sensitive peptide.

In various embodiments, the cationic peptide only includes R, K, and/or H residues; and/or the anionic peptide only includes D and/or E residues. In one embodiment, the cationic peptide is a polyR peptide. In another embodiment, the anionic peptide is a polyE peptide. In various embodiments, the cationic domain and/or the anionic domain may include D amino acids, or may be exclusively D amino acids. In some embodiments, the detectable marker may be a fluorophore selected from the group consisting of fluorescein phosphoramidides, rhodamine, polymethadine dye derivative, phosphores, Texas red, green fluorescent protein, Cy3, Cy5, and Cy7. In other embodiments, the fluorophore is a fluorophore component of a fluorophore-quencher pair, and the anionic peptide linked to the quencher component of the fluorophore-quencher pair. In other embodiments, the protease-cleavable peptide comprises the sequence selected from the group consisting of SEQ ID NO:1-196. In one embodiment, the AZP comprises the sequence selected from the group consisting of SEQ ID NO:167-196.

In another aspect, the disclosure provides methods for localizing protease activity in a tissue section, comprising
(a) contacting a biological tissue section that is not formalin preserved with a probe, comprising the general formula X1-X2-X3, wherein
one of X1 and X3 is a cationic peptide linked to a detectable marker and the other is a anionic peptide;
X2 is a protease-sensitive peptide, wherein proteases present in the biological tissue section cleave the protease-sensitive peptide, resulting in binding of the detectably tagged cationic peptide to the tissue; and
(b) detecting signal from the biological tissue section, wherein the fluorescence indicates where protease activity is present in the biological tissue section.

### DESCRIPTION OF THE FIGURES

### Example 1 Figure Legends

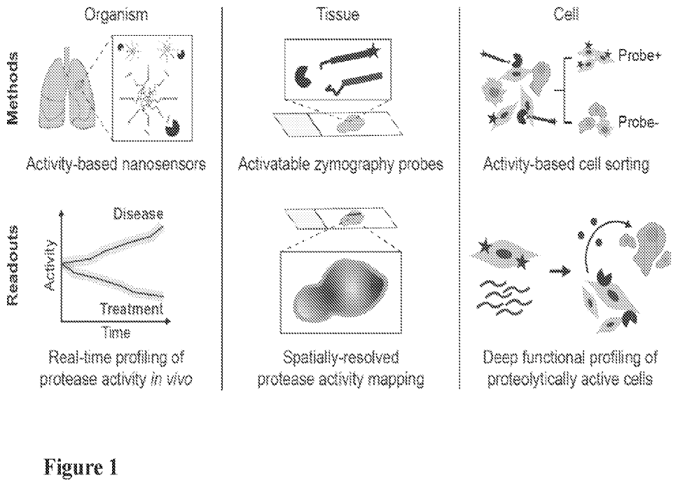

FIG. 1. Multiscale profiling of protease activity in cancer. (Top) Methods for profiling protease activity across the organism, tissue, and cellular scales. Noninvasive activity-based nanosensors can be translated into activatable probes for in situ zymography and activity-based cell sorting. (Bottom) Method readouts enable noninvasive, real-time monitoring of in vivo protease activity over tumor progression and treatment response; spatially-resolved activity mapping of the TME; and single-cell isolation and multimodal characterization of proteolytically active cells.

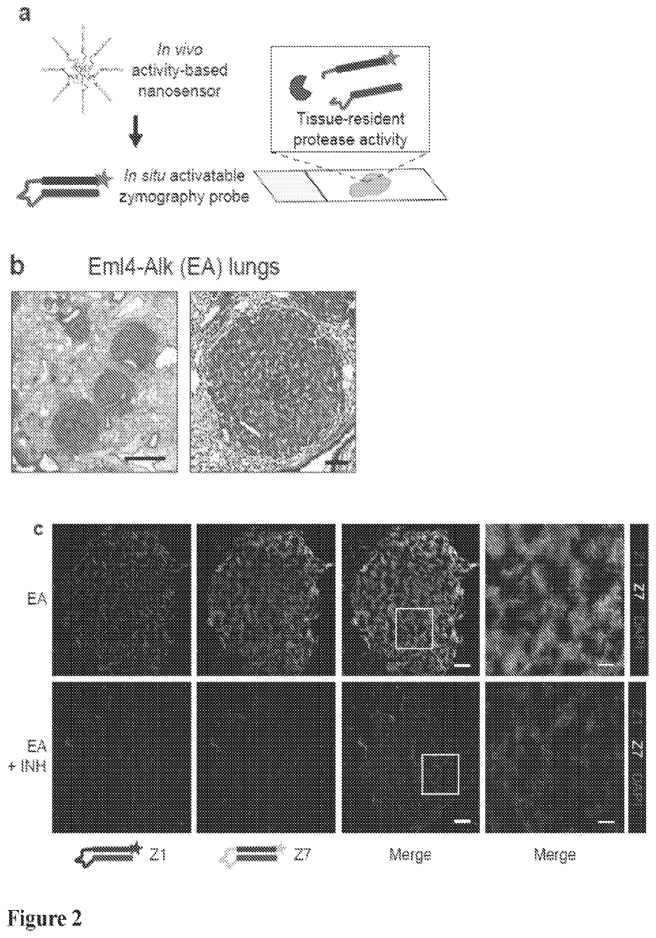

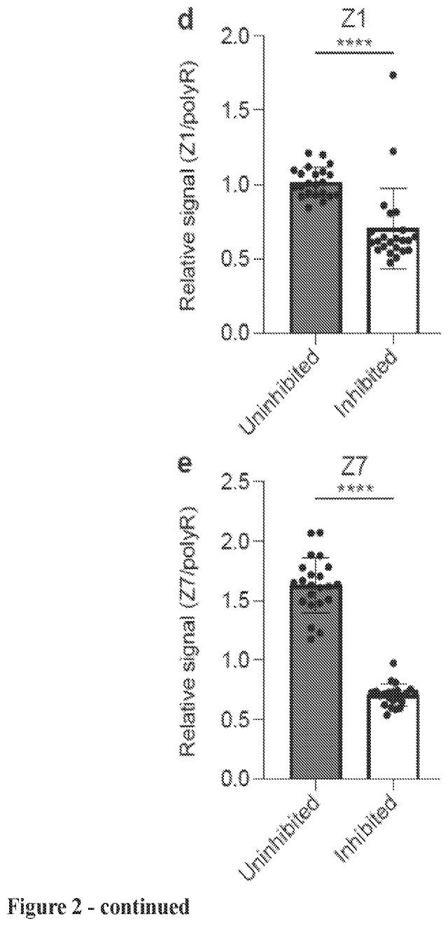

FIG. 2. Multiplexed AZPs reveal spatially distinct protease activity patterns. (a) Substrates nominated from in vivo profiling were translated into in situ AZPs to measure and spatially localize tissue-resident enzyme activity in frozen tissue sections. (b) AZPs were applied to fresh-frozen lung tissue sections from Eml4-Alk (EA) tumor-bearing mice. Haematoxylin and eosin (H&E) stains of representative Eml4-Alk tumors. Scale bars=500 μm (left), 100 μm (right). (c) Application of a multiplexed AZP cocktail of Z1 and Z7, either uninhibited (EA) or with broad-spectrum protease inhibitors (EA+INH), to Eml4-Alk lung tissue sections. Scale bars=100 μm. Higher magnification images of boxed regions show localization patterns from multiplexed AZP labeling. Scale bars=25 μm. (d-e) Quantification of relative Z1 (d) and Z7 (e) intensity, normalized to polyR binding control signal on a per-cell basis across Eml4-Alk tumors, either in the absence of protease inhibitors (Uninhibited; n=22 tumors) or in the presence of a broad-spectrum cocktail of protease inhibitors (Inhibited; n=23 tumors) (mean±s.d.; unpaired two-sided t-test, ****P<0.0001).

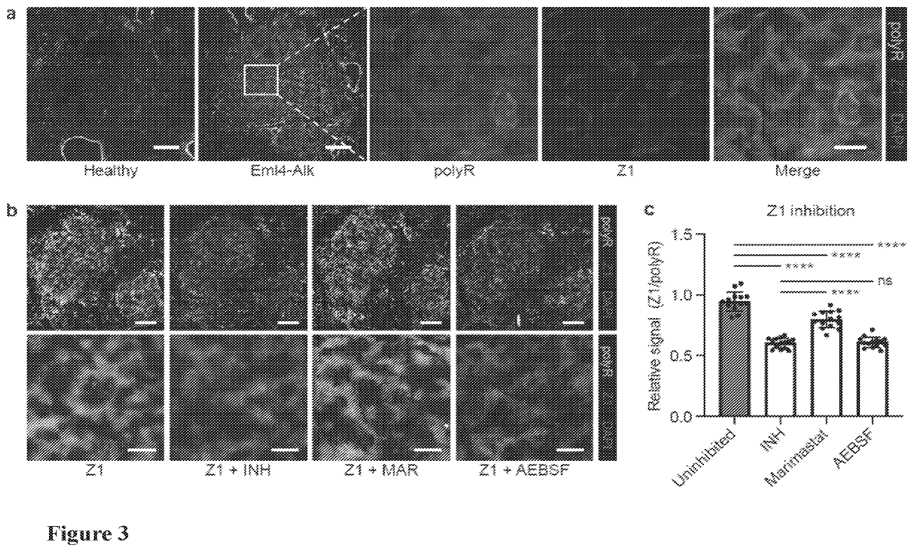

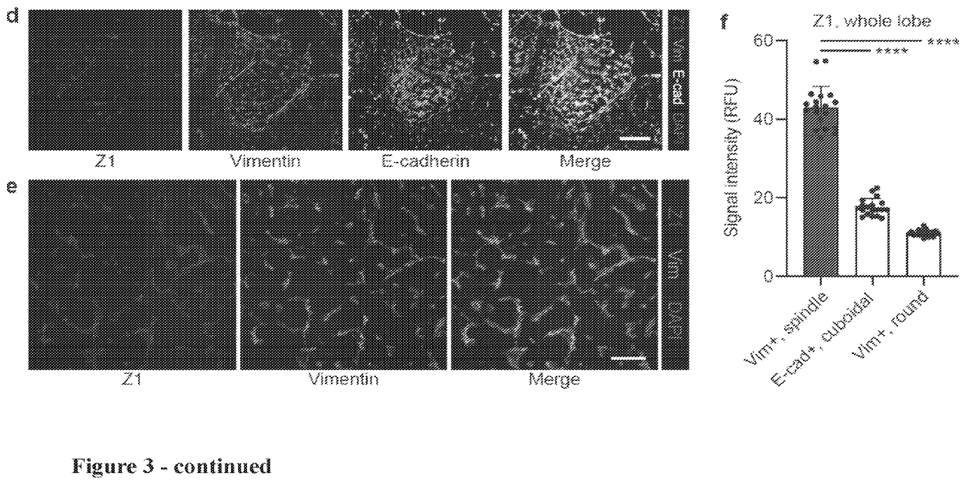

FIG. 3. AZPs identify mechanistic class- and cell type-specific protease activity. (a) Staining of lung tissue sections from healthy control and Eml4-Alk mice with Z1, polyR, and DAPI counterstain. Higher magnification images show staining in a representative Eml4-Alk tumor region. Scale bars=200 μm, 50 μm (lower and higher magnification, respectively). (b) Application of Z1 to Eml4-Alk lung tissue sections, either alone (Z1) or in the presence of inhibitors: a broad-spectrum cocktail of protease inhibitors (Z1+INH), the MMP inhibitor marimastat (Z1+MAR), or the serine protease inhibitor AEBSF (Z1+AEBSF). Sections were stained with a polyR binding control and counterstained with DAPI. Scale bars=200 μm (top), 50 μm (bottom). (c) Quantification of relative Z1 intensity, normalized to polyR signal, from Eml4-Alk tumors, either in the absence of protease inhibitors (Uninhibited), or in the presence of INH, MAR, or AEBSF (n=14 tumors; mean±s.d.; unpaired two-sided t-test,****P<0.0001, nsP=0.7127). (d) Application of Z1 to Eml4-Alk lung tissues sections with co-staining for vimentin and E-cadherin. Scale bar=200 μm. (e) Higher-magnification images of a second tumor region, independent of that shown in (d), showing Z1 and vimentin stains. Scale bar=50 μm. (f) Quantification of Z1 staining intensity for per-tumor cell populations, across an entire lung lobe (n=19 tumors, with intensities averaged across all cells in a tumor; mean±s.d.; paired two-sided t-test, ****P<0.0001).

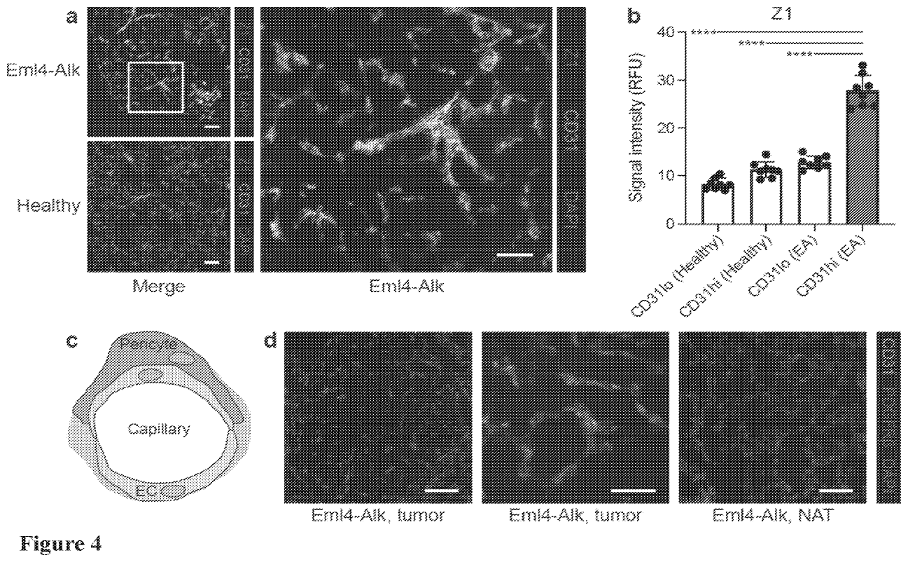

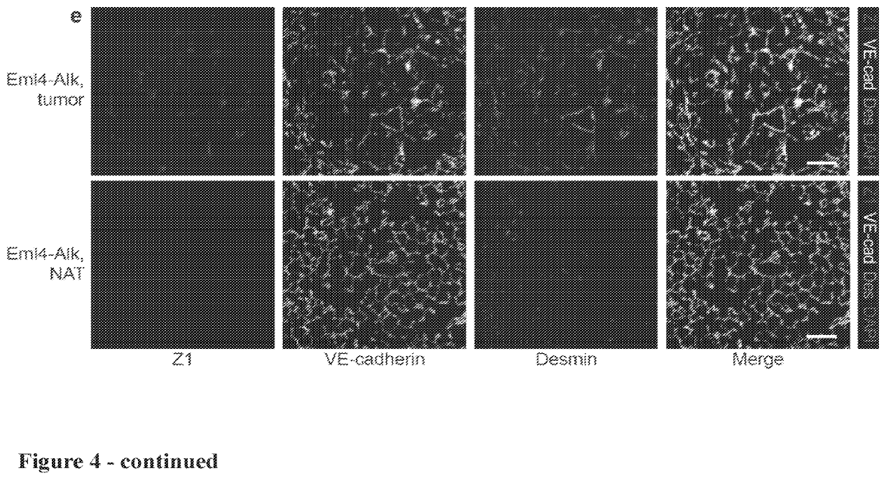

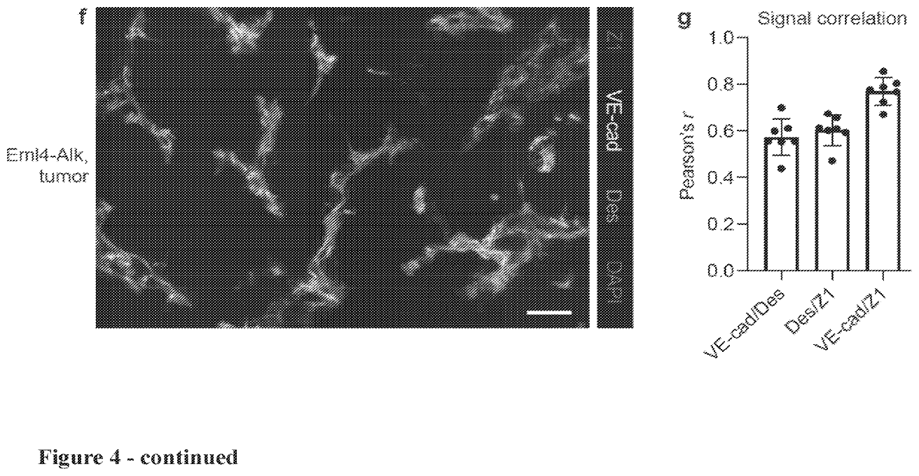

FIG. 4. Z1 localizes to pericyte-invested vasculature in Eml4-Alk tumors. (a) Application of Z1 to Eml4-Alk and healthy lung tissue sections with co-staining for CD31. Scale bars=100 μm, 50 μm (lower and higher magnification, respectively). (b) Quantification of Z1 staining intensity in CD31-low (CD30lo) and CD31-high (CD31hi) cells (n=8 regions per condition; mean f s.d.; one-way ANOVA with Tukey correction for multiple comparisons, ****P<0.0001). (c) Capillary vessels are lined by endothelial cells (EC); pericytes support and wrap around endothelial cells. (d) Staining for endothelial cells (CD31) and pericytes (PDGFRβ) in formalin-fixed, paraffin-embedded Eml4-Alk lung tissue sections, with images from representative tumor (left, middle) and normal adjacent tissue (NAT; right) regions shown. Scale bar=100 μm, 20 μm (lower and higher magnification, respectively). (e) Application of Z1 to Eml4-Alk lung tissues with co-staining for VE-cadherin (VE-cad) and desmin (Des). Scale bars=100 μm. (f) Higher magnification image of representative Eml4-Alk tumor region showing localization of Z1, VE-cadherin, and desmin. Scale bar=20 μm. (g) Pearson's correlation of pixel-wise signal intensities for each pairwise combination of Z1, VE-cadherin, and desmin (n=7 tumors; mean+s.d.).

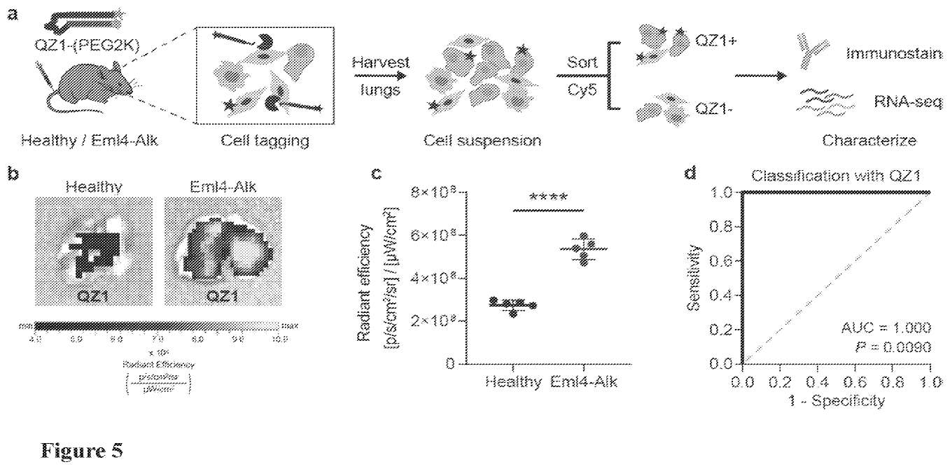

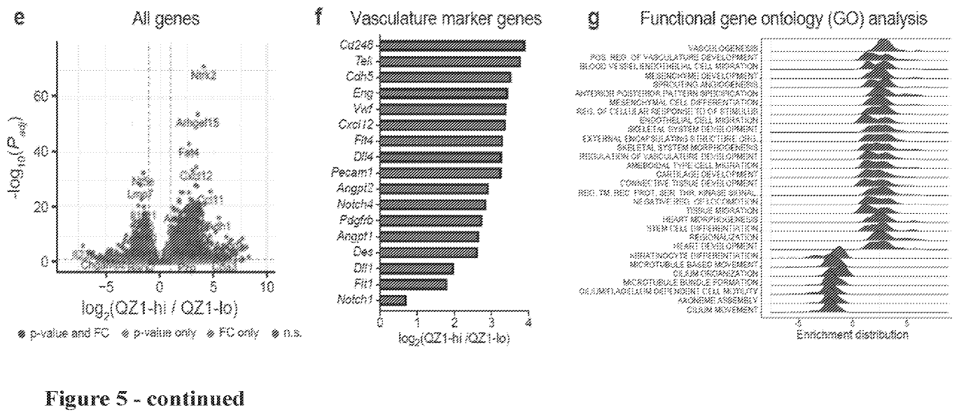

FIG. 5. Activity-based cell sorting enables multimodal phenotypic characterization of proteolytically active cells of the tumor microenvironment. (a) The quenched probe QZ1-(PEG2K), consisting of a Cy5-tagged polyR (star+rectangle) and quencher-tagged polyE (hexagon+rectangle), was administered intravenously into age-matched healthy and Eml4-Alk mice. Lungs were excised, imaged, and dissociated into single cell suspension. Cells were sorted on Cy5 fluorescence and then characterized via immunostaining and bulk RNA-seq. (b) Images of representative lungs from healthy and Eml4-Alk mice 2 hours after QZ1-(PEG2K) administration. (c) Quantification of epifluorescence radiant efficiency from experiment in (b) (n=5 mice per group; mean±s.d.; unpaired two-sided t-test, ****P<0.0001). (d) ROC curve showing performance of QZ1-(PEG2K) signal in discriminating healthy from Eml4-Alk lung explants (AUC=1.000, 95% confidence interval 1.000-1.000; P=0.0090 from random classifier shown in dashed line). (e) Differential expression analysis of RNA-seq data from sorted QZ1-hi and QZ1-lo cells. Each point represents one gene and is colored according to the significance level for that gene. Significance was calculated by Wald test followed by adjustment for multiple hypotheses using the Benjamini-Hochberg correction. Dotted line is at Padj=0.05.\

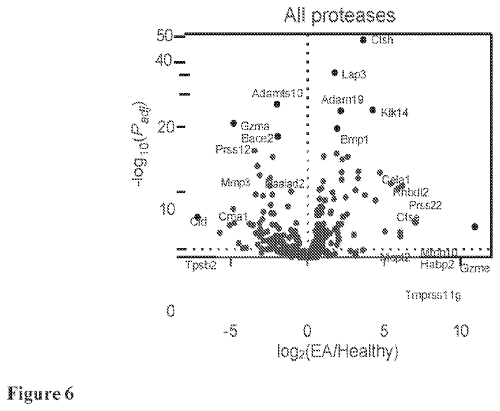

FIG. 6. Proteases are differentially expressed in the Eml4-Alk mouse model of NSCLC. An existing bulk RNA-seq dataset from the Eml4-Alk model [26] was analyzed to identify endoproteases differentially expressed between Eml4-Alk mice (EA) and healthy controls (Healthy). Each point represents one protease gene. Significance was calculated by Wald test followed by adjustment for multiple hypotheses using the Benjamini-Hochberg correction. Dotted line is at P_{adj}=0.05.

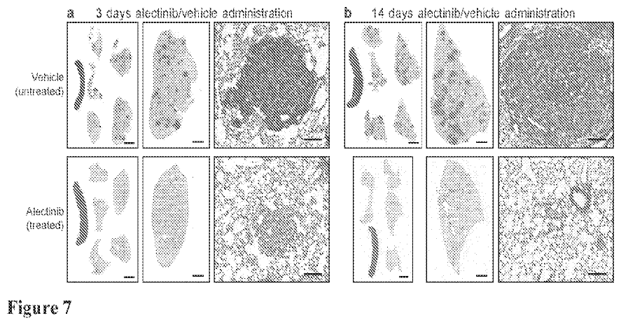

FIG. 7. Alectinib treatment induces tumor regression in the Eml4-Alk model. (a, b) Images of representative lung sections and tumors (4 whole-lung sections per group; 5 lobes per lung set; 10-20 tumor regions per lobe) from vehicle control (untreated) and alectinib treated-mice, 3 days (a) and 14 days (b) post initiation of alectinib or vehicle administration. Scale bars=2 mm, 1 mm, 100 μm for all lobes, single lobe, and individual tumors, respectively.

FIG. 8. AZPs nominated from in vivo profiling label Eml4-Alk tumors in a protease-dependent manner. (a-c) Individual application of Z1 (a), Z7 (b), or Z10 (c), either alone or with broad-spectrum protease inhibitors (+INH), to Eml4-Alk lung tissue sections. Sections were counterstained with DAPI Boxes show regions in higher magnification images on right. Scale bars=200 μm, 50 μm (lower and higher magnification, respectively). Tumors shown are representative of all tumors in assayed tissue, with 7-10 tumors per lung tissue section. (d) Application of a multiplexed AZP cocktail of Z1, Z7, and Z10, either uninhibited (EA) or with broad-spectrum protease inhibitors (EA+INH), to Eml4-Alk lung tissue sections. Scale bars=100 μm.

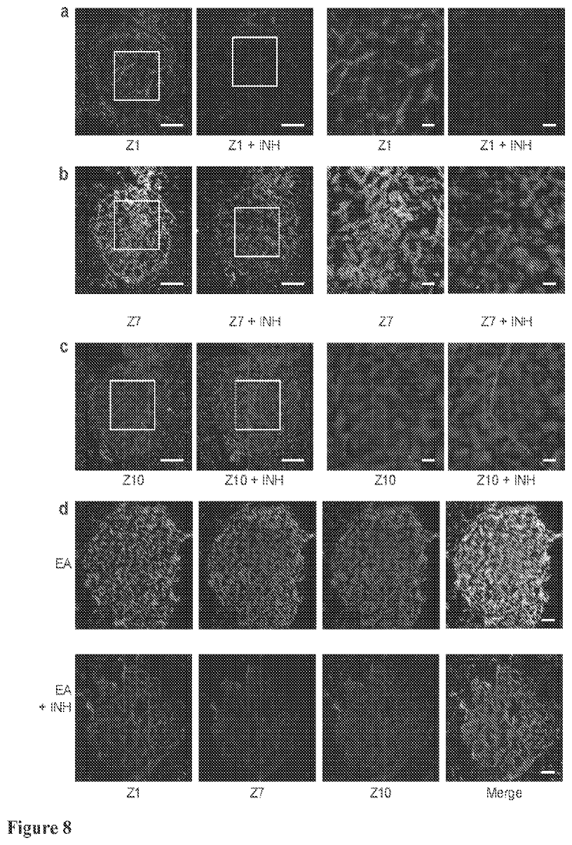

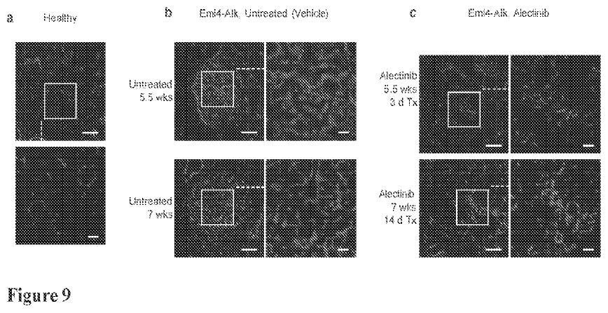

FIG. 9. Z1 labeling of Eml4-Alk tumors is abrogated by treatment with alectinib. (a) Staining of representative section from healthy lungs with Z1. (b) Staining of representative tumor regions from untreated Eml4-Alk mice at 5.5 (top) and 7 (bottom) weeks post tumor induction. (c) Staining of representative tumor regions from alectinib-treated Eml4-Alk mice at 5.5 (top) and 7 (bottom) weeks post tumor induction, corresponding to 3 (top) and 14 (bottom) days post initiation of alectinib treatment. All sections were counterstained with DAPI. Scale bars=200 μm, 50 μm (lower and higher magnification, respectively). Tumors shown are representative of all tumors in assayed tissue, with 7-10 tumors per lung tissue section.

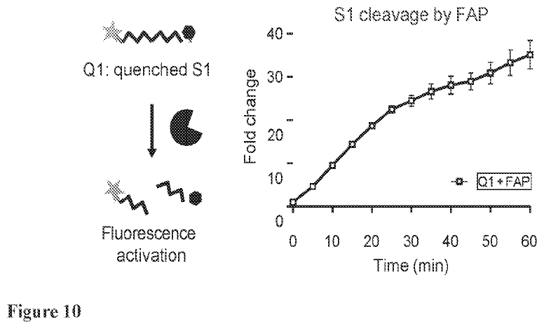

FIG. 10. Fibroblast activation protein (FAP) cleaves S1 in vitro. The quenched probe Q1, incorporating the substrate S1, was incubated with recombinant FAP, and fluorescence activation was monitored over time (n=2 replicates; mean t s.d.).

FIG. 11. Z1 localization pattern is not due to nonspecific binding. (a, b) Tissue labeling and localization of intact (a) or FAP-precleaved Z1 (PC; b), together with free polyR control, within Eml4-Alk tumors. Probes were incubated at 37° C. for 4 hours to allow cleavage of intact Z1 by endogenous, tissue-resident proteases. Scale bars=100 μm, 25 μm (lower and higher magnification, respectively). Tumors shown are representative of all tumors in assayed tissue, with 7-10 tumors per lung tissue section.

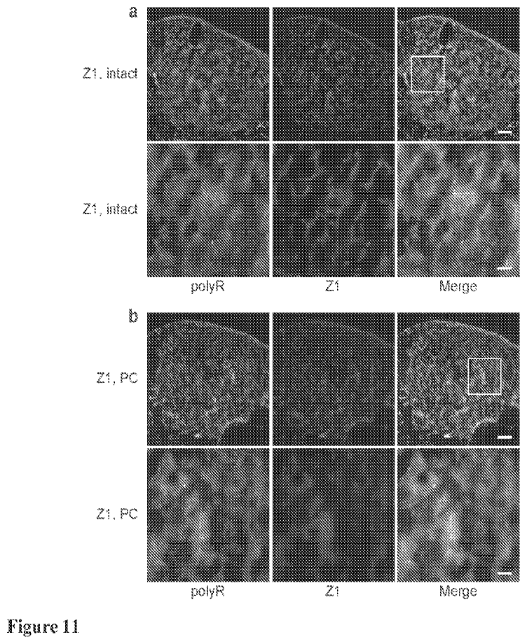

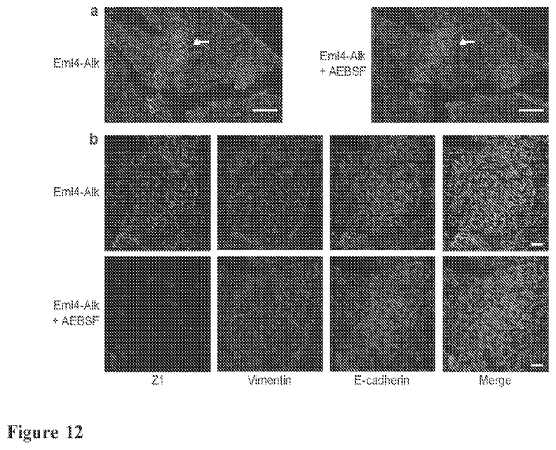

FIG. 12. Z1 staining is abrogated by serine protease inhibitor in tissues co-stained for vimentin and E-cadherin. (a) Staining of Eml4-Alk lungs with Z1, together with the mesenchymal marker vimentin and the epithelial marker E-cadherin, with or without the serine protease inhibitor AEBSF. Scale bars=500 μm. (b) Z1, vimentin, and E-cadherin staining in representative Eml4-Alk tumor regions, indicated by arrows in (a). Scale bars=100 μm. Tumors shown are representative of all tumors in assayed tissue, with 7-10 tumors per lung tissue section.

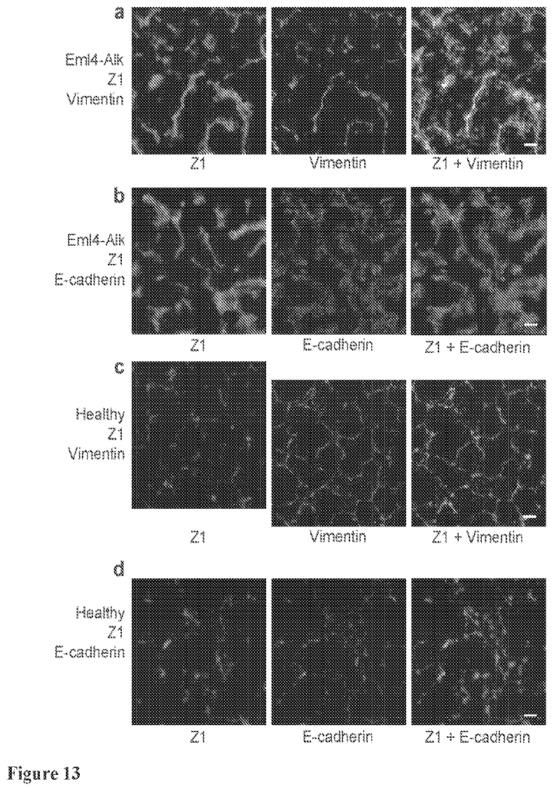

FIG. 13. Z1 localization pattern aligns closely with vimentin. (a) Co-staining of representative Eml4-Alk tumor region with Z1 together with vimentin. (b) Co-staining of representative Eml4-Alk tumor region with Z1 together with E-cadherin. (c) Co-staining of healthy lungs with Z1 together with vimentin. (d) Co-staining of healthy lungs with Z1 together with E-cadherin. Scale bars=25 μm. Tumors shown are representative of all tumors in assayed tissue, with 7-10 tumors per lung tissue section. Representative healthy regions are representative of four independent healthy lung sections assayed.

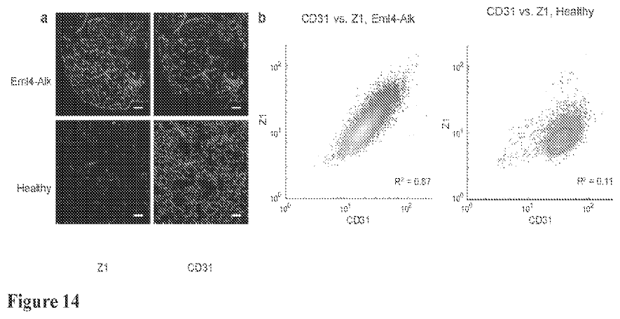

FIG. 14. Z1 staining correlates with CD31 staining in Eml4-Alk tumors. (a) Application of Z1 to lung tissue sections from Eml4-Alk and healthy control mice, together with the endothelial marker CD31. Scale bars=100 μm. Tumors shown are representative of all tumors in assayed tissue, with 7-10 tumors per lung tissue section. Representative healthy regions are representative of four independent healthy lung sections assayed. (b) Cell-by-cell quantification and correlation of Z1 and CD31 fluorescence intensity in Eml4-Alk (left) and healthy (right) lung tissue sections (R²=0.67. R²=0.11 for Eml4-Alk and healthy, respectively).

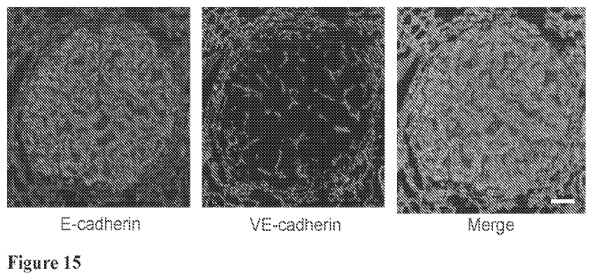

FIG. 15. Eml4-Alk tumors stain positively for E-cadherin and VE-cadherin. Immunofluorescence staining for E-cadherin and the endothelial marker VE-cadherin in formalin-fixed, paraffin-embedded Eml4-Alk lung tissue sections, with images from a representative tumor shown. Sections were counterstained with DAPI. Scale bar=50 μm. Tumors shown are representative of all tumors in assayed tissue, with approximately 20-30 tumors per lung tissue section.

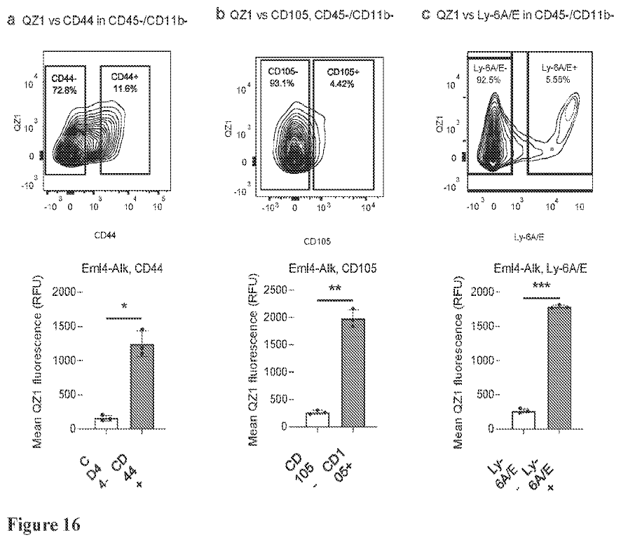

FIG. 16. QZ1 signal in Eml4-Alk tumors is increased in cell populations positive for mesenchymal markers. (a-c) Flow cytometry plot (top) and quantification (bottom) of Cy5 (QZ1) fluorescence intensity in CD45−, CD11b− cells from Eml4-Alk lungs, gated by the cell surface markers CD44 (a), CD105 (b), and Ly-6A/E (c) (n=3 biological replicates; mean s.d.; paired two-sided t-test, *P=0.0120 for CD44, **P=0.00285 for CD105, ***P=0.000374 for Ly-6A/E).

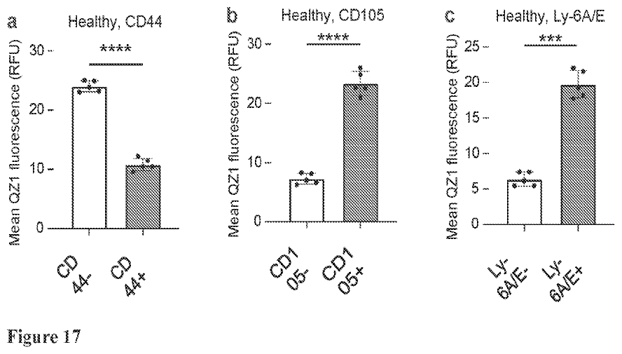

FIG. 17. Cells positive for mesenchymal markers in healthy lungs exhibit variable QZ1 labeling. (a-c) Quantification of Cy5 (QZ1) fluorescence intensity in CD45−, CD11b− cells from healthy lungs, gated by the cell surface markers CD44 (a), CD105 (b), and Ly-6A/E (c) (n=3 biological replicates; mean s.d.; paired two-sided t-test, ****P<0.0001 for CD44, ****P<0.0001 for CD105, ***P=0.0002 for Ly-6A/E.

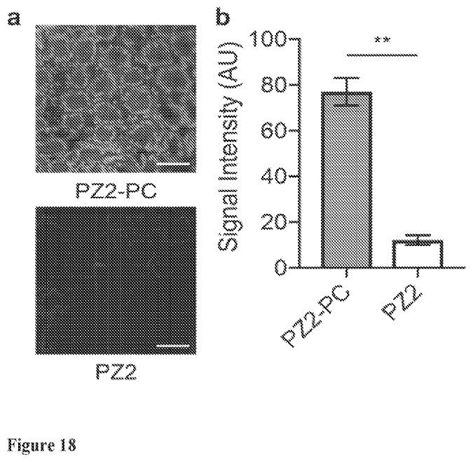

FIG. 18. Cleavage-dependent tissue labeling with AZPs. (a) Staining of frozen colon sections with PZ2 pre-cleaved with PRSS3 (PZ2-PC; top) and intact PZ2 (PZ2; bottom). Scale bars 50 μm. (b) Quantification of signal intensity in sections stained with PZ2-PC or PZ2 (**P=0.0049, n=2 technical replicates).

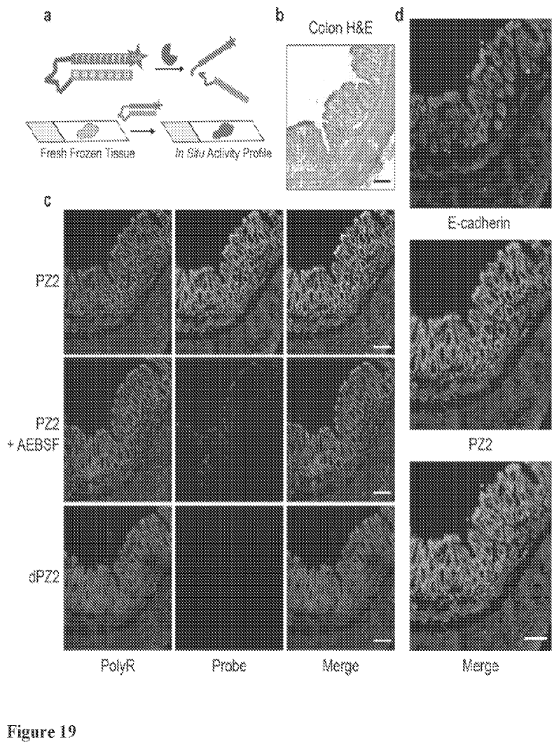

FIG. 19. In situ localization of protease activity with AZPs. (a) Design of AZPs, wherein a poly-arginine domain with fluorophore tag (star) is linked to a poly-glutamic acid domain via protease-cleavable linker. Application of AZP to fresh frozen tissue section allows for spatial localization of protease activity directly in situ. (b) Hematoxylin and eosin (H&E) staining of a region of healthy mouse colon. (c) Staining of frozen colon sections with PolyR-Cy7 (left column) and either uPA-activatable PZ2-Cy5 (top and middle rows; PZ2) or d-stereoisomer dPZ2-Cy5 (bottom row; dPZ2). Top and middle rows show staining of consecutive sections without (top) and with (bottom) the serine protease inhibitor AEBSF. (d) Region of colon tissue showing cell-level colocalization of activated PZ2 with staining for the epithelial cell marker E-cadherin. All scale bars=100 μm

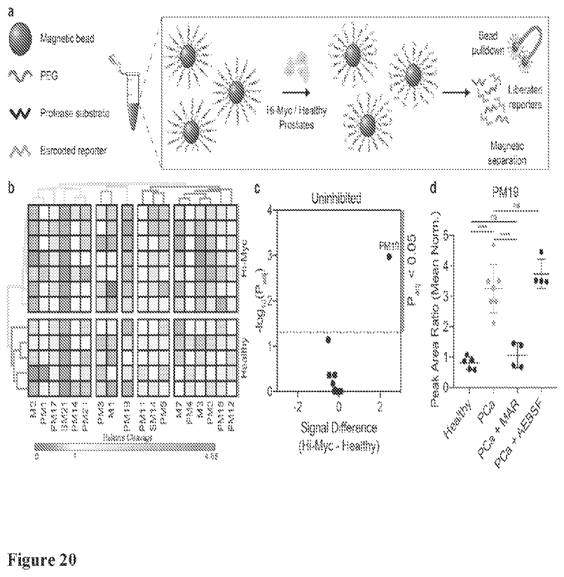

FIG. 20. Discovery of MMP dysregulation in Hi-Myc PCa GEMM via multiplexed substrate screen. (a) Protease-cleavable peptide substrates with barcoded reporters are conjugated onto the surface of a magnetic bead. Following incubation with prostate homogenates and proteolysis, magnetic pull-down enables isolation and identification of cleavage products. (b) Hierarchical clustering of cleavage data from peptide screen against homogenates of prostates from healthy and Hi-Myc GEMM mice. (c) Mean normalized cleavage product concentrations in Hi-Myc (n=7) and healthy (n=5) prostate homogenates were compared, and −log₁₀(P_{adj}) was plotted against relative signal between Hi-Myc and healthy. Significance was calculated by two-tailed t-test followed by adjustment for multiple hypotheses using the Holm-Sidak method. Dotted line is at P_{adj}=0.05. (d) Quantification of PM19 cleavage for healthy homogenates and Hi-Myc homogenates, either uninhibited (PCa), in the presence of the MMP inhibitor marimastat (PCa+MAR), or in the presence of the serine protease inhibitor AEBSF (PCa+AEBSF) (one-way ANOVA with Tukey's correction for multiple comparisons, ****P<0.0001, ns=not significant).

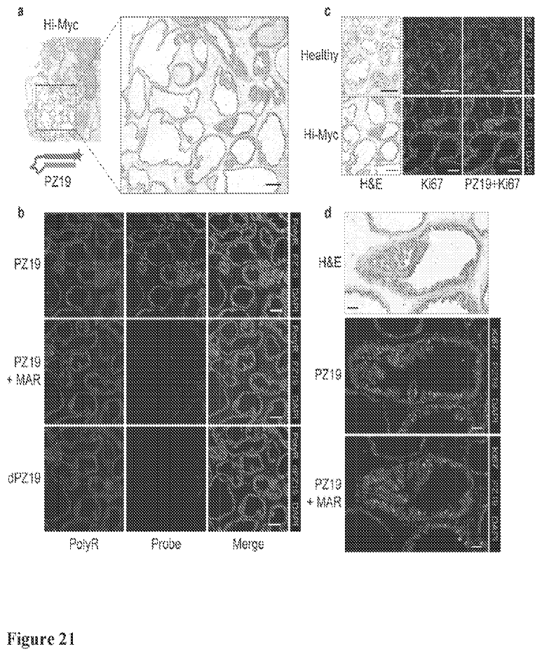

FIG. 21. AZPs enable localization of MMP dysregulation in Hi-Myc PCa GEMM. (a) H&E staining of Hi-Myc prostate tissue showing epithelial glands that appear histologically normal. The MMP-responsive AZP PZ19 was applied to Hi-Myc prostates to measure and localize substrate-specific cleavage events in situ. Scale bar=200 μm. (b) Staining of consecutive sections of the region of Hi-Myc tissue from (a) with PolyR-Cy7 and either MMP-activatable PZ19-FAM (top and middle rows; PZ19) or d-stereoisomer dPZ19-FAM (bottom row; dPZ19). Top and middle rows show staining of consecutive sections without (top) and with (middle) MAR. Sections were counterstained with DAPI. Scale bars=200 μm. (c) Application of PZ19 to prostate tissues of healthy (top) and Hi-Myc (bottom) mice with co-staining for the proliferation marker Ki67. Scale bars=200 μm. (d) Higher-magnification image of glandular region of Hi-Myc prostate showing co-localization of MMP-activated PZ19 and Ki67. A consecutive section was stained with PZ19 and Ki67 in the presence of MAR. Scale bars=50 μm.

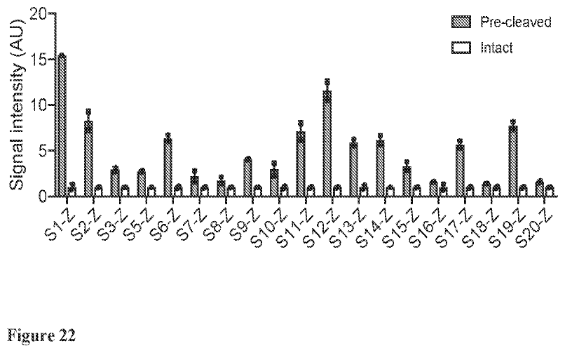

FIG. 22. Characterization of AZP library. AZPs, either intact or with linkers pre-cleaved by a cognate recombinant protease, were applied to fresh-frozen colon tissue for 30 minutes, and fluorescent signal intensity of bound probes was quantified (n=1-2 replicates per probe; mean±s.d.).

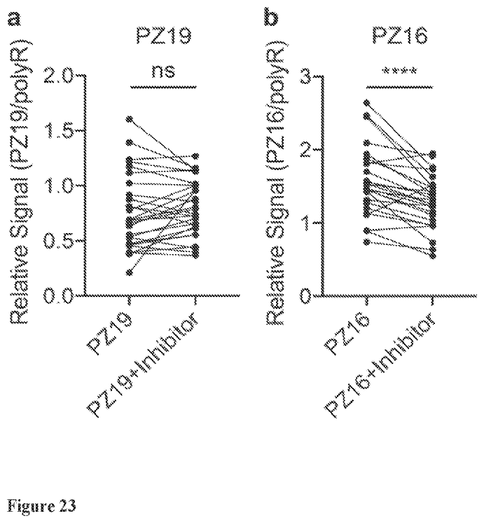

FIG. 23. Evaluation of MMP-responsive PZ19 and PZ16 on human PCa TMA. (a, b) Quantification of relative PZ19 (a) and PZ16 (b) signal, measured as fold change between AZP signal intensity and polyR binding control, following application to a human PCa TMA, with and without protease inhibitors (two-tailed paired t-test; ****P<0.0001).

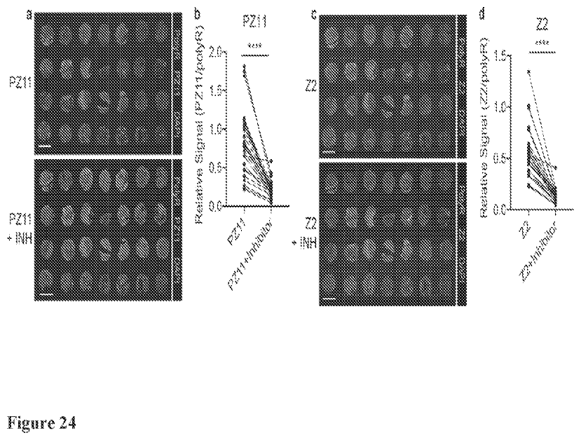

FIG. 24. AZPs are responsive to proteolytic activity in human tissues. (a, c) Application of PZ11 (a) and Z2 (c) AZPs to a human PCa tissue microarray (TMA) consisting of 24 prostate adenocarcinoma samples and 4 normal prostate samples (top). A consecutive TMA was stained with PZ11 (a) or Z2 (c) along with a cocktail of protease inhibitors (bottom). Scale bars=2 mm. (b, d) Quantification of relative PZ11 (b) and Z2 (d) signal, measured as fold change between PZ11 or Z2 and polyR binding control signal intensities, for TMA cores (n=28) incubated with PZ11 or Z2, with and without protease inhibitors (two-tailed paired t-test, ****P<0.0001).

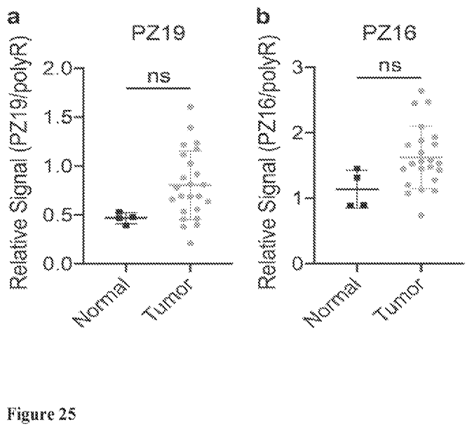

FIG. 25. Evaluation of discriminative power of PZ19 and PZ16 on human PCa TMA. (a, b) Quantification of relative PZ19 (a) and PZ16 (b) signal from normal (n=4) and PCa tumor (n=24) cores (Student's t-test, ns=not significant). Relative signal is measured as fold change between AZP and polyR binding control signal intensities.

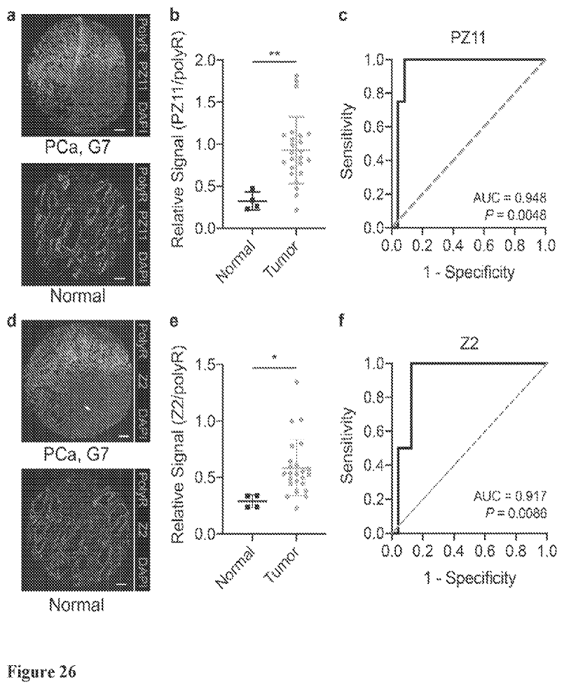

FIG. 26. AZPs enable ex vivo classification of human PCa. (a) Higher-magnification image of select cores from PZ11-stained TMA (FIG. 23a) showing a patient sample of Gleason 7 PCa (top) and a sample of normal prostate (bottom). Scale bars=200 μm. (b) Quantification of relative PZ11 signal from normal (n=4) and PCa tumor (n=24) cores (Student's t-test, **P=0.0063). Relative PZ11 signal is measured as fold change between PZ11 and polyR binding control signal intensities. (c) Receiver-operating characteristic (ROC) curve by relative AZP signal (PZ11/polyR) discriminated normal from PCa tumor cores with an AUC of 0.948 (95% confidence interval 0.8627-1.000; P=0.0048 from random classifier shown in dashed line). (d) Higher-magnification image of select cores from Z2-stained TMA (FIG. 23c) showing a patient sample of Gleason 7 PCa (top) and a sample of normal prostate (bottom). Scale bars=200 μm. (e) Quantification of relative Z2 signal from normal (n=4) and PCa tumor (n=24) cores (Student's t-test, *P=0.0284). Relative Z2 signal is measured as fold change between Z2 and polyR binding control signal intensities. (f) Receiver-operating characteristic (ROC) curve by relative AZP signal (Z2/polyR) discriminated normal from PCa tumor cores with an AUC of 0.917 (95% confidence interval 0.8103-1.000; P=0.0086 from random classifier shown in dashed line).

### DETAILED DESCRIPTION

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

All embodiments of any aspect of the invention can be used in combination, unless the context clearly dictates otherwise.

In a first aspect, the disclosure provides methods for activity based cell sorting, comprising
(a) contacting a biological sample with an activatable zymography probe (AZP), wherein the AZP comprises the general formula X1-X2-X3, wherein
   (i) one of X1 and X3 is a cationic peptide linked to a fluorophore, and the other is an anionic peptide; and
   (ii) X2 is protease-cleavable peptide,
wherein degradation of the protease-cleavable peptide by proteases present in the biological sample liberates the fluorophore-linked cationic peptide so that it binds cells at or near a site of protease degradation of the protease-cleavable peptide to produce fluorophore-tagged cells; and
(b) isolating the fluorophore tagged cells from the biological sample by fluorescence activated cell sorting (FACS).

As described in the examples, the methods of the disclosure permit isolating cells based on their protease activity.

The methods may utilize any suitable biological sample, including but not limited to urine, fecal, blood, saliva, or bodily samples; a whole organism (including but not limited to test animals), a tissue section, live/fresh or frozen; a tissue specimen; any vitro or ex vivo cell cultures in 2D, 4D, etc., or any other suitable sample.

In one embodiment, the cationic peptide linked to the fluorophore is located amino-terminal to X2 (i.e.: it is X1); in another embodiment, the cationic peptide linked to the fluorophore is located carboxy-terminal to X2 (i.e.: it is X3).

Any suitable cationic peptide and anionic peptide may be used in the methods of the disclosure. Any sufficiently positively-charged peptide interfaced with a reciprocally negatively-charged peptide, where the electrostatic interaction is strong enough to form a complex may be used. In various embodiments, the cationic peptide may be poly Arginine (polyR), poly Lysine (polyK), poly Histidine (polyHis), or any combination of R, K, and/or H residues. In further embodiments, the anionic peptide may be poly glutamic acid (polyE), poly aspartic acid (polyD), or any combination of E and D residues. The cationic peptide and anionic peptide may include other residues so long as they are sufficiently positively-charged peptide interfaced with a reciprocally negatively-charged peptide, where the electrostatic interaction is strong enough to form a complex. In one embodiment, the cationic peptide only includes R, K, and/or H residues. In another embodiment, the anionic peptide only includes D and/or E residues.

The cationic and anionic peptides may independently be of any length that provides an electrostatic interaction strong enough to form a complex between the two. In one embodiment, the cationic and anionic peptides are independently at least 2, 3, 4, 5, 6, 7, 8, 9, 10, or more amino acids in length. In another embodiment, the cationic and anionic peptides are independently between 2-30 amino acids in length; in other embodiments, independently between 3-30, 4-30, 5-30, 6-30, 7-30, 8-30, 3-25, 4-25, 5-25, 6-25, 7-25, 8-25, 3-20, 4-20, 5-20, 6-20, 7-20, 8-20, 3-15, 4-15, 5-15, 6-15, 7-15, 8-15, 3-10, 4-10, 5-10, 6-10, 7-10, or 8-10 amino acids in length.

The cationic and anionic peptides may be the same length or different lengths, so long as the charges of each are sufficiently close and oppositely signed such that an electrostatic complex can be maintained, as will be understood by those of skill in the art based on the teachings herein.

Any fluorophore may be used as deemed suitable for an intended use. In various non-limiting embodiments, the fluorophore may comprise fluorescein phosphoramidides such as fluoreprime (Pharmacia, Piscataway, N.J.), fluoredite (Millipore, Bedford, Mass.), FAM (ABI, Foster City, Calif.), rhodamine, polymethadine dye derivative, phosphores, Texas red, green fluorescent protein, Cy3, Cy5, and Cy7.

In one embodiment, the fluorophore may be a fluorophore component of a fluorophore-quencher pair, and the anionic peptide linked to the quencher component of the fluorophore-quencher pair. Any fluorophore-quencher pair may be used, as suitable for an intended use. In one such embodiment, the fluorophore-quencher pair is selected from the group consisting of:
5-Carboxyfluorescein (5-FAM) and CPQ2;
FAM and DABCYL;
Cy5 and QSY21; and
Cy3 and QSY7

In another embodiment, the anionic peptide may be linked to a second fluorophore to permit ratiometric imaging with dual fluorophores. Any fluorophore can be linked to the anionic peptide, as suitable for an intended use. In one such embodiment, one of the cationic peptide or the anionic peptide is linked to Cy5 and the other is linked to Cy7.

Any suitable protease-cleavable peptide comprises may be used as suitable for an intended purpose. In various non-limiting embodiments, the protease-cleavable peptide comprises the sequence selected from the group consisting of SEQ ID NOS: 1-166, wherein:
Residues in lower case are D amino acids;
Orn is ornithine;
[Cha] is 3-cyclohexylalanine;
[Cys(Me)] is methyl-cysteine;
[Nval] is Norvaline;
[Phe(homo)] is homo-phenylalanine;
[Pip] is pipecolinic acid;
[Tic] is 1,2,3,4-tetrahydroisoquinoline-3-carboxylic acid;
[Nle(O-Bzl)] is Hydroxynorleucine(Bzl);
Met(O)2] is Dioxymethionine/Methionine sulfone;
[Oic] is octahydroindole-2-carboxylic acid; and
[Abu] is 2-Aminobutanoic acid.

See Table 1 and, for example, US20200096514, incorporated by reference herein in its entirety).

TABLE 1

| SEQ ID | |
|---|---|
| Number | Sequence |
| 1 | GGPQGIWGQG |
| 2 | GGLGPKGQTGG |
| 3 | GILSRIVGGG |
| 4 | GGPQGIWGQK |
| 5 | GGPVGLIGK |
| 6 | GGPVPLSLVMK |
| 7 | GGPLGLRSWK |
| 8 | GGPLGVRGKK |
| 9 | GGfPRSGGGK |
| 10 | GGLGPKGQTGK |
| 11 | GGGSGRSANAKG |
| 12 | GKPISLISSG |
| 13 | GILSRIVGGG |
| 14 | GSGSKIIGGG |
| 15 | GGPLGMRGG |
| 16 | GAPFEMSAG |
| 17 | GRQRRVVGG |
| 18 | GRQARAVGG |
| 19 | GRRARVVGG |
| 20 | GPLGLRSWG |
| 21 | GWEAVRSAMWG |
| 22 | GWIGFRNAGAG |
| 23 | GPLGFRVG |
| 24 | GPLGLRG |
| 25 | GATPKIFNTEG |
| 26 | GETRIIKGFEG |
| 27 | GPLALWARG |
| 28 | GALVPRGSAG |
| 29 | GGLVPRGSG |
| 30 | GRQARQVGG |
| 31 | GLQARVVGG |
| 32 | GGGSGRSANAKG |
| 33 | GGVPRGG |
| 34 | GRQSRIVGGG |
| 35 | GILSRIVGGG |
| 36 | GGRKRKVGGSG |
| 37 | GDEKGKARDFFG |
| 38 | GKPISLISSG |
| 39 | GLAQAFRSG |
| 40 | GRPLALWESQG |
| 41 | GG[Orn]RSHPFTLYTA |
| 42 | GRQRRALEKG |
| 43 | GIQQRSLGGG |
| 44 | GLRGGKGGPPPPG |
| 45 | GGGAPFEMSA |
| 46 | GPRPFNYLG |
| 47 | GAGPRKAAKG |
| 48 | GAKIRKAKKG |
| 49 | GAKIRGQAKG |
| 50 | GNSGRAVTYG |
| 51 | GTYSRSRYLG |
| 52 | GRPKPQQFWG |
| 53 | GMAALIVRPDLG |
| 54 | GGRIFLRTAG |
| 55 | GSGDRMWggG |
| 56 | GSGERMMGgG |
| 57 | GsDDRRAGgG |
| 58 | GKLRVVGGHPG |
| 59 | GMAALITRPDFG |
| 60 | GELIQRNLSPAG |
| 61 | GSNLTRIVGGQG |
| 62 | GYQIKPLKSTDG |
| 63 | GNIPMGLLYNKG |
| 64 | GPQGRIVGG |
| 65 | GIKPRIVGG |
| 66 | GPMKRLTLG |
| 67 | GGLGPKGQTG |
| 68 | GGAGGAQMGA |
| 69 | GGATDVTTTP |
| 70 | GGLSLPETGE |
| 71 | GSPLAQAVRSSS |
| 72 | GSSMAQTLTLRSSS |
| 73 | GGGPLGLARG |
| 74 | GMERMGG |
| 75 | GGGGPGG |
| 76 | GEWWMDYQG |
| 77 | GFLRRQFKVVT |
| 78 | GVGRPEWWMDG |
| 79 | GLGALLRVKRLE |
| 80 | GLASASTMDG |
| 81 | GGKGRGLSLG |
| 82 | GHGDQMAQKS |
| 83 | GRAEQQRLKG |
| 84 | GRAEQQRLG |
| 85 | GDQMAQKSQG |
| 86 | GAIEFDSG |
| 87 | GGPQGIWGQ |
| 88 | GGPVPLSLVM |
| 89 | GGPLGVRGK |
| 90 | GGEPRSGGG |
| 91 | GSGSKIIGGG |
| 92 | GPLGMRG |
| 93 | GAPFEMSAG |
| 94 | GGP[Cha]G[Cys(Me)]HAGC |
| 95 | GGPVGLIG |
| 96 | GGAAEAISDA |
| 97 | GGAQPDALNV |
| 98 | GGDIVTVANA |
| 99 | GGDLGLKSVP |
| 100 | GGDVMASQKR |
| 101 | GGESDELQTI |
| 102 | GGFHPLHSKI |
| 103 | GGGHARLVHV |
| 104 | GGHIANVERV |
| 105 | GGKAAATQKK |
| 106 | GGLATASTMD |
| 107 | GGNLAGILKE |
| 108 | GGNPGMSEPV |
| 109 | GGPFGCHAK |
| 110 | GGPLGLRWW |
| 111 | GGQMGVMQGV |
| 112 | GGQTCKCSCK |
| 113 | GGQWAGLVEK |
| 114 | GGRPAVMTSP |
| 115 | GGTLRELHLD |
| 116 | GGTPPPSQGK |
| 117 | GGTSEDLVVQ |
| 118 | GGVWAAEAIS |
| 119 | GRPKPVE[Nval]WRKG |
| 120 | GHSSKLQG |
| 121 | GSSQYSSNGG |
| 122 | GQKGRYKQEG |
| 123 | GGKAFRRSGG |
| 124 | GAANLTRG |
| 125 | GGGELRG |
| 126 | GLAQA[Phe(homo)]RSG |
| 127 | GSPLAQAVRSSG |
| 128 | GPVPLSLVMG |
| 129 | GSQPRIVGGG |
| 130 | GSYRIFGG |
| 131 | RPKPVEGG |
| 132 | GRRRGGAAN[C(OMeBzl)]RMGG |
| 133 | GVPLSLTMGG |
| 134 | GAPFEFSAG |
| 135 | GRPLALWRSG |
| 136 | GRPLALEESQG |
| 137 | GRPLALWRSQG |
| 138 | GRNALAVERTASG |
| 139 | GRPKPVE[Nval]WRG |
| 140 | GSGSNPYKYTAG |
| 141 | GSGSNPYGYTAG |
| 142 | GSGTLSELHTAG |
| 143 | GSGTISHLHTAG |
| 144 | GSG[Orn]RSHP[Phe(homo)]TLYTAG |
| 145 | GSG[Orn]RSHG[Phe(homo)]FLYTAG |
| 146 | GSGESLAYYTAG |
| 147 | GSGHMHAALTAG |
| 148 | GSGKGPRQITAG |
| 149 | GSGPLFYSVTAG |
| 150 | GSGRSENIRTAG |
| 151 | GGPWGIWGQG |
| 152 | GGf[Pip]RSGGG |
| 153 | GGf[Pip]KSGGG |
| 154 | GGHPGGPQG |
| 155 | GGGVFRMLSVG |
| 156 | GGGLFRSLSSG |
| 157 | GGGLLYGKGG |
| 158 | Ggy[Tic]TNG |
| 159 | GGIPSIQSRGLG |
| 160 | GGNLARALKQTIG |
| 161 | GGHMVQHLIQWHG |
| 162 | GGPRAAA[Phe(homo)]TSPG |
| 163 | GGTGPPGYTG |
| 164 | GGTGLPVYQG |
| 165 | GG[Nle(O-Bzl)][Met(O)2][Oic][Abu] |
| 166 | GGAAFAG; |

The AZPs may further comprise any other functional units as deemed appropriate for an intended use. In one embodiment, the AZP further comprises a stabilization domain. Any suitable stabilization domain may be used, including but not limited to a targeting peptide and polyethylene glycol (PEG). The stabilization domain may be linked to the AZP using any technique, including but not limited to adding a cysteine residue to the AZP to facilitate binding to the stabilization domain via cysteine-maleimide chemistry.

In another embodiment, the amino acid residues in the AZP may be modified as suitable for an intended purpose. In one embodiment, the AZP may include D amino acids for increased stability. In one such embodiment, the cationic domain and/or the anionic domain include D amino acids, or are exclusively D amino acids.

In one embodiment:
the cationic domain is polyR in D amino acid form;
the anionic domain is polyE in D amino acid form; and
the fluorophore-quencher pair is Cy5 and QSY21.

In other embodiments, the AZP comprises:

(QSY21)-eeeeeeeee-c(PEG2K)-oGGPQGIWGQG-rrrrrrrrr-k(Cy5) (SEQ ID NO: 167),
wherein:
lowercase amino acid residues are in D amino acid form;
PEG2K: (poly)ethylene-glycol, MW 2000 g/mol; and
o is 5-amino-3-oxopentanoyl, or another residue for physical distancing and/or stabilization, or is absent.

In other embodiments, the AZP probe may comprise:

(SEQ ID NO: 168)
UeeeeeeeeXGGPQGIWGQGrrrrrrrrrX-k(Cy5);
(SEQ ID NO: 169)
UeeeeeeeeXGGLGPKGQTGGrrrrrrrrrX-k(Cy3);
(SEQ ID NO: 170)
UeeeeeeeeXGILSRIVGGGrrrrrrrrrX-k(FAM);
(SEQ ID NO: 167)
(QSY21)-eeeeeeeee-c(PEG2K)-oGGPQGIWGQG-
rrrrrrrrr-k(Cy5);

wherein:
lowercase amino acid residues are D-amino acids;
PEG2K is (poly)ethylene-glycol, MW 2000 g/mol;
“o” is 5-amino-3-oxopentanoyl or another residue for physical distancing and/or stabilization, or is absent;
U is succinoyl or is absent; and
X is 6-aminohexanoyl or is absent.

In one embodiment, “o”, “U”, and X are present.

In other embodiments, the AZP probe may comprise:

(SEQ ID NO: 171; S1-Z in FIG. 22)
U-eeeeeeee-X-GGPQGIWGQG-rrrrrrrrr-X-K(Cy3);
(SEQ ID NO: 172; S2-Z in FIG. 22)
U-eeeeeeee-X-GGLVPRGSGG-rrrrrrrrr-X-K(Cy5);
(SEQ ID NO: 173; S3-Z in FIG. 22)
U-eeeeeeee-X-GGPVGLIGG-rrrrrrrrr-X-K(5FAM);
(SEQ ID NO: 174)
U-eeeeeeee-X-GPLGVRGKG-rrrrrrrrr-X-K(5FAM);
(SEQ ID NO: 175; S5-Z in FIG. 22)
U-eeeeeeee-X-GRQRRALEKG-rrrrrrrrr-X-K(Cy3);
(SEQ ID NO: 176; S6-Z in FIG. 22)
U-eeeeeeee-X-GSGRSANAG-rrrrrrrrr-X-K(Cy5);
(SEQ ID NO: 177; S7-Z in FIG. 22)
U-eeeeeeee-X-GKPISLISSG-rrrrrrrrr-X-K(5FAM);
(SEQ ID NO: 178; S8-Z in FIG. 22)
U-eeeeeeee-X-GILSRIVGGG-rrrrrrrrr-X-K(5FAM);
(SEQ ID NO: 179; S9-Z in FIG. 22)
U-eeeeeeee-X-GRPKPVE(Nval)WRKG-rrrrrrrrr-X-K
(5FAM);
(SEQ ID NO: 180; S10-Z in FIG. 22)
U-eeeeeeee-X-GIQQRSLGGG-rrrrrrrr-X-K(5FAM);
(SEQ ID NO: 181; S11-Z in FIG. 22)
U-eeeeeeee-X-GGGVPRGGG-rrrrrrrr-X-K(5FAM);
(SEQ ID NO: 182; S12-Z in FIG. 22)
U-eeeeeeee-X-GSGSKIIGGG-rrrrrrrrr-X-K(Cy3);
(SEQ ID NO: 183; S13-Z in FIG. 22)
U-eeeeeeee-X-GGAANLTRGG-rrrrrrrrr-X-K(Cy3);
(SEQ ID NO: 184; S14-Z in FIG. 22)
U-eeeeeeee-X-GLAQAPhe(homo)RSG-rrrrrrrrr-X-K(Cy3);
(SEQ ID NO: 185; S15-Z in FIG. 22)
U-eeeeeeee-X-GSPLAQAVRSSG-rrrrrrrrr-X-K(Cy3);
(SEQ ID NO: 186; S16-Z in FIG. 22)
U-eeeeeeee-X-GPVPLSLVMG-rrrrrrrrr-X-K(5FAM);
(SEQ ID NO: 187; S17-Z in FIG. 22)
U-eeeeeeee-X-GSQPRIVGGG-rrrrrrrrr-X-K(Cy5);
(SEQ ID NO: 188; S18-Z in FIG. 22)
U-eeeeeeee-X-GGGHARLVHVG-rrrrrrrrr-X-K(Cy3);
(SEQ ID NO: 189; S19-Z in FIG. 22)
U-eeeeeece-X-GGLGPKGQTGG-rrrrrrrrr-X-K(Cy3);
(SEQ ID NO: 190; S20-Z in FIG. 22)
U-eeeeeeee-X-GGQTCKCSCKG-rrrrrrrrr-X-K(Cy5);
(SEQ ID NO: 191)
U-eeeeeeee-X-GsGrsanaG-rrrrrrrrr-X-K(Cy5);
(SEQ ID NO: 192)
(QSY21)-eeeeeeeee-c-o-GSGRSANAG-rrrrrrrrr-K(Cy5);
(SEQ ID NO: 193)
(QSY21)-eeeeeeeee-c-o-GsGrsanaG-rrrrrrrrr-K(Cy5);
(SEQ ID NO: 194)
U-eeeeeeee-X-Gpvp U-eeeeeeee-X-GRQRRALEKG-
rrrrrrrrr-X-K lslvmG-rrrrrrrrr-X-K(5FAM);
(SEQ ID NO: 195)
(QSY21)-eeeeeeeee-c-o-GPVPLSLVMG-rrrrrrrrr-K(Cy5);
(SEQ ID NO: 196)
(QSY21)-eeeeeeeee-c-o-GpvplslvmG-rrrrrrrrr-K(Cy5);

wherein lowercase amino acid residues are D-amino acids; “o” is 5-amino-3-oxopentanoyl or another residue for physical distancing and/or stabilization, or is absent, and U is succinoyl or is absent; and X is 6-aminohexanoyl or is absent. In one embodiment, “o”, “U”, and X are present.

The methods comprise isolating the fluorophore tagged cells from the biological sample by fluorescence activated cell sorting (FACS), using standard techniques. Exemplary FACS techniques are as described in the examples.

The isolated cells may then be characterized in any way deemed appropriate for an intended purpose. In one embodiment, the further characterizing comprises gene expression analysis of the isolated tagged cells, such as described in the examples.

In a second aspect, the disclosure provides probes that can be used, for example, in the methods of the invention. All aspects of the AZPs as disclosed above are equally applicable to the probes of the disclosure. In one embodiment, the probe comprises the general formula X1-X2-X3, wherein
one of X1 and X3 is a cationic peptide linked to a detectable marker and the other is a anionic peptide;
X2 is a protease-sensitive peptide.

In one embodiment of the probes, the cationic peptide linked to the fluorophore is located amino-terminal to X2 (i.e.: it is X1); in another embodiment, the cationic peptide linked to the fluorophore is located carboxy-terminal to X2 (i.e.: it is X3).

Any suitable cationic peptide and anionic peptide may be used in the probes of the disclosure. Any sufficiently positively-charged peptide interfaced with a reciprocally negatively-charged peptide, where the electrostatic interaction is strong enough to form a complex may be used. In various embodiments, the cationic peptide may be poly Arginine (polyR), poly Lysine (polyK), poly Histidine (polyHis), or any combination of R, K, and/or H residues. In further embodiments, the anionic peptide may be poly glutamic acid (polyE), poly aspartic acid (polyD), or any combination of E and D residues. The cationic peptide and anionic peptide may include other residues so long as they are sufficiently positively-charged peptide interfaced with a reciprocally negatively-charged peptide, where the electrostatic interaction is strong enough to form a complex. In one embodiment, the cationic peptide only includes R, K, and/or H residues. In another embodiment, the anionic peptide only includes D and/or E residues.

The cationic and anionic peptides may independently be of any length that provides an electrostatic interaction strong enough to form a complex between the two. In one embodiment, the cationic and anionic peptides are independently at least 2, 3, 4, 5, 6, 7, 8, 9, 10, or more amino acids in length. In another embodiment, the cationic and anionic peptides are independently between 2-30 amino acids in length; in other embodiments, independently between 3-30, 4-30, 5-30, 6-30, 7-30, 8-30, 3-25, 4-25, 5-25, 6-25, 7-25, 8-25, 3-20, 4-20, 5-20, 6-20, 7-20, 8-20, 3-15, 4-15, 5-15, 6-15, 7-15, 8-15, 3-10, 4-10, 5-10, 6-10, 7-10, or 8-10 amino acids in length.

The cationic and anionic peptides may be the same length or different lengths, so long as the charges of each are sufficiently close and oppositely signed such that an electrostatic complex can be maintained, as will be understood by those of skill in the art based on the teachings herein.

The cationic and anionic peptides may independently be of any length that provides an electrostatic interaction strong enough to form a complex between the two. In one embodiment, the cationic and anionic peptides are independently at least 2, 3, 4, 5, 6, 7, 8, 9, 10, or more amino acids in length. In another embodiment, the cationic and anionic peptides are independently between 2-30 amino acids in length; in other embodiments, independently between 3-30, 4-30, 5-30, 6-30, 7-30, 8-30, 3-25, 4-25, 5-25, 6-25, 7-25, 8-25, 3-20, 4-20, 5-20, 6-20, 7-20, 8-20, 3-15, 4-15, 5-15, 6-15, 7-15, 8-15, 3-10, 4-10, 5-10, 6-10, 7-10, or 8-10 amino acids in length.

Any detectable marker may be used in the probes of this second aspect as deemed suitable for an intended use, including but not limited to an epitope (i.e.: detectable by an antibody recognizing the epitope), a DNA barcode, a fluorescent marker, etc In one embodiment, the detectable marker is a fluorophore. Any fluorophore may be used as deemed suitable for an intended use. In various non-limiting embodiments, the fluorophore may comprise fluorescein phosphoramidides such as fluoreprime (Pharmacia, Piscataway, N.J.), fluoredite (Millipore, Bedford, Mass.), FAM (ABI, Foster City, Calif.), rhodamine, polymethadine dye derivative, phosphores, Texas red, green fluorescent protein, Cy3, Cy5, and Cy7.

In one embodiment, the fluorophore may be a fluorophore component of a fluorophore-quencher pair, and the anionic peptide linked to the quencher component of the fluorophore-quencher pair. Any fluorophore-quencher pair may be used, as suitable for an intended use. In one such embodiment, the fluorophore-quencher pair is selected from the group consisting of:
5-Carboxyfluorescein (5-FAM) and CPQ2;
FAM and DABCYL;
Cy5 and QSY21; and
Cy3 and QSY7

In another embodiment of the probes, the anionic peptide may be linked to a second fluorophore to permit ratiometric imaging with dual fluorophores. Any fluorophore can be linked to the anionic peptide, as suitable for an intended use. In one such embodiment, one of the cationic peptide or the anionic peptide is linked to Cy5 and the other is linked to Cy7.

Any suitable protease-cleavable peptide comprises may be used in the probes as suitable for an intended purpose. In various non-limiting embodiments, the protease-cleavable peptide comprises the sequence selected from the group consisting SEQ ID NO:1-166 (See Table 1 and, for example, US20200096514, incorporated by reference herein in its entirety).

The probes may further comprise any other functional units as deemed appropriate for an intended use. In one embodiment, the probe further comprises a stabilization domain. Any suitable stabilization domain may be used, including but not limited to polyethylene glycol (PEG). The stabilization domain may be linked to the probe using any technique, including but not limited to adding a cysteine residue to the probe to facilitate binding to the stabilization domain.

In another embodiment, the amino acid residues in the probe may be modified as suitable for an intended purpose. In one embodiment, the probe may include D amino acids for increased stability. In one such embodiment, the cationic domain and/or the anionic domain include D amino acids, or are exclusively D amino acids.

In one embodiment:
the cationic domain is polyR in D amino acid form;
the anionic domain is polyE in D amino acid form; and
the fluorophore-quencher pair is Cy5 and QSY21.

In other embodiments, the probe comprises:

(SEQ ID NO: 167)
(QSY21)-eeeeeeeee-c(PEG2K)-oGGPQGIWGQG-rrrrrrrrr-
k(Cy5),

wherein:
lowercase amino acid residues are in D amino acid form,
PEG2K: (poly)ethylene-glycol, MW 2000 g/mol; and
o is 5-amino-3-oxopentanoyl or another residue for physical distancing and/or stabilization, or is absent.

In other embodiments, the AZP probe may comprise:

(SEQ ID NO: 168)
UeeeeeeeeXGGPQGIWGQGrrrrrrrrrX-k(Cy5);
(SEQ ID NO: 169)
UeeeeeeeeXGGLGPKGQTGGrrrrrrrrrX-k(Cy3);
(SEQ ID NO: 170)
UeeeeeeeeXGILSRIVGGGrrrrrrrrrX-k(FAM);
(SEQ ID NO: 167)
(QSY21)-eeeeeeeee-c(PEG2K)-oGGPQGIWGQG-
rrrrrrrrr-k(Cy5);

wherein lowercase amino acid residues are D-amino acids; PEG2K is (poly)ethylene-glycol, MW 2000 g/mol; “o” is 5-amino-3-oxopentanoyl or another residue for physical distancing and/or stabilization, or is absent, U is succinoyl or is absent; and X is 6-aminohexanoyl or is absent. In one embodiment, “o”, “U”, and X are present.

In other embodiments, the AZP probe may comprise:

(SEQ ID NO: 171)
U-eeeeeeee-X-GGPQGIWGQG-rrrrrrrrr-X-K(Cy3);
(SEQ ID NO: 172)
U-eeeeeeee-X-GGLVPRGSGG-rrrrrrrrr-X-K(Cy5);
(SEQ ID NO: 173)
U-eeeeeeee-X-GGPVGLIGG-rrrrrrrrr-X-K(5FAM);
(SEQ ID NO: 174)
U-eeeeeeee-X-GPLGVRGKG-rrrrrrrrr-X-K(5FAM);
(SEQ ID NO: 175)
U-eeeeeeee-X-GRQRRALEKG-rrrrrrrrr-X-K(Cy3);
(SEQ ID NO: 176)
U-eeeeeeee-X-GSGRSANAG-rrrrrrrrr-X-K(Cy5);
(SEQ ID NO: 177)
U-eeeeeeee-X-GKPISLISSG-rrrrrrrrr-X-K(5FAM);
(SEQ ID NO: 178)
U-eeeeeeee-X-GILSRIVGGG-rrrrrrrrr-X-K(5FAM);
(SEQ ID NO: 179)
U-eeeeeeee-X-GRPKPVE(Nval)WRKG-rrrrrrrrr-X-K
(5FAM);
(SEQ ID NO: 180)
U-eeeeeeee-X-GIQQRSLGGG-rrrrrrrr-X-K(5FAM);
(SEQ ID NO: 181)
U-eeeeeeee-X-GGGVPRGGG-rrrrrrrr-X-K(5FAM);
(SEQ ID NO: 182)
U-eeeeeeee-X-GSGSKIIGGG-rrrrrrrrr-X-K(Cy3);
(SEQ ID NO: 183)
U-eeeeeeee-X-GGAANLTRGG-rrrrrrrrr-X-K(Cy3);
(SEQ ID NO: 184)
U-eeeeeeee-X-GLAQAPhe(homo)RSG-rrrrrrrrr-X-K(Cy3);
(SEQ ID NO: 185)
U-eeeeeeee-X-GSPLAQAVRSSG-rrrrrrrrr-X-K(Cy3);
(SEQ ID NO: 186)
U-eeeeeeee-X-GPVPLSLVMG-rrrrrrrrr-X-K(5FAM);
(SEQ ID NO: 187)
U-eeeeeeee-X-GSQPRIVGGG-rrrrrrrrr-X-K(Cy5);
(SEQ ID NO: 188)
U-eeeeeeee-X-GGGHARLVHVG-rrrrrrrrr-X-K(Cy3);
(SEQ ID NO: 189)
U-eeeeeece-X-GGLGPKGQTGG-rrrrrrrrr-X-K(Cy3);
(SEQ ID NO: 190)
U-eeeeeeee-X-GGQTCKCSCKG-rrrrrrrrr-X-K(Cy5);
(SEQ ID NO: 191)
U-eeeeeeee-X-GsGrsanaG-rrrrrrrrr-X-K(Cy5);
(SEQ ID NO: 192)
(QSY21)-eeeeeeeee-c-o-GSGRSANAG-rrrrrrrrr-K(Cy5);
(SEQ ID NO: 193)
(QSY21)-eeeeeeeee-c-o-GsGrsanaG-rrrrrrrrr-K(Cy5);
(SEQ ID NO: 194)
U-eeeeeeee-X-Gpvp U-eeeeeeee-X-GRQRRALEKG-
rrrrrrrrr-X-K lslvmG-rrrrrrrrr-X-K(5FAM);
(SEQ ID NO: 195)
(QSY21)-eeeeeeeee-c-o-GPVPLSLVMG-rrrrrrrrr-K(Cy5);
(SEQ ID NO: 196)
(QSY21)-eeeeeeeee-c-o-GpvplslvmG-rrrrrrrrr-K(Cy5);

wherein lowercase amino acid residues are D-amino acids; “o” is 5-amino-3-oxopentanoyl or another residue for physical distancing and/or stabilization, or is absent; U is succinoyl or is absent; and X is 6-aminohexanoyl or is absent. In one embodiment, “o”, “U”, and X are present.

In a third aspect, the disclosure provides methods for localizing protease activity in a tissue section, comprising
(a) contacting a biological tissue section that is not formalin preserved with a probe, comprising the general formula X1-X2-X3, wherein
one of X1 and X3 is a cationic peptide linked to a detectable marker and the other is a anionic peptide;
X2 is a protease-sensitive peptide, wherein proteases present in the biological tissue section cleave the protease-sensitive peptide, resulting in binding of the detectably tagged cationic peptide to the tissue; and
(b) detecting signal from the biological tissue section, wherein the fluorescence indicates where protease activity is present in the biological tissue section.

The detectable maker may be any marker deemed appropriate for an intended use. In various embodiments, the marker may be an epitope (i.e.: detectable by an antibody recognizing the epitope), a DNA barcode, a fluorescent marker, etc. In one embodiment, the probe is a probe described herein.

The tissue section may be any tissue section, so long as it is not formalin preserved. In various embodiments, the tissue section is live/fresh or is frozen.

In one embodiment, the probe comprises the probe of any embodiment or combination of embodiment of the second aspect of the disclosure.

### Example 1

Diverse processes in cancer are mediated by enzymes, which most proximally exert their function through their activity. High-fidelity methods to profile enzyme activity are therefore critical to understanding and targeting the pathological roles of enzymes in cancer. Here, we present an integrated set of methods for measuring specific protease activities across scales, and deploy these methods to study treatment response in an autochthonous model of A/k-mutant lung cancer. We leverage multiplexed nanosensors and machine learning to analyze in vivo protease activity dynamics in lung cancer, identifying significant dysregulation that includes enhanced cleavage of a peptide, S1, which rapidly returned to healthy levels with targeted therapy. Through direct on-tissue localization of protease activity, we pinpoint S1 cleavage to the tumor vasculature. To link protease activity to cellular function, we design a high-throughput method to isolate and characterize proteolytically active cells, uncovering a pro-angiogenic phenotype in S1-cleaving cells. These methods provide a framework for functional, multiscale characterization of protease dysregulation in cancer.

### Introduction

Diverse processes in tumor progression not only rely on changes in abundance, but on the dynamics in activity of biomolecules. Methods to quantitatively track protein activity within the cellular, tissue, and organismic contexts are therefore critical to advance understanding of cancer biology and to design next-generation precision cancer medicines.

Methods to analyze enzyme activity at the organism, tissue, and cellular scales and the biological insights that they provide could open new diagnostic and therapeutic avenues in cancer. Recent years have seen a push to develop biosensors that measure biomolecular activity in vivo to generate synthetic signals that can be read out noninvasively. However, such in vivo readouts have largely treated the body as a black box, sacrificing information on spatial localization within the tumor microenvironment (TME), precluding dissection of phenotypic heterogeneity at the single-cell level, and thus reducing biological interpretation. Therefore, there remains a need for methods capable of generating and unifying molecular activity measurements across biological scales.

In this work, we present an integrated set of methods to profile protease activity in cancer across the organism, tissue, and cellular scales (FIG. 1). In the in vivo setting, we leverage multiplexed protease-responsive nanosensors together with machine learning to noninvasively detect and longitudinally monitor disease progression in preclinical mouse models. To explore tissue-level organization within the TME, we establish a multiplexed assay for on-tissue spatial localization of protease activity against target peptide substrates. Finally, to link protease activity to other measurement modalities at the cellular scale, we design a single-cell method, termed activity-based cell sorting, that uses peptide probes and flow cytometry to sort individual cells based on their associated enzymatic activity.

We unified these methods into a hierarchical framework (FIG. 1) and applied it to study tumor progression and early drug response in an autochthonous mouse model of Alk-mutant non-small-cell lung cancer (NSCLC). We uncovered significant shifts in protease activity that occur after targeted therapy with the ALK inhibitor alectinib. Spatial and single-cell profiling linked a treatment-responsive activity signature to pericytes and endothelial cells of the angiogenic tumor vasculature, suggesting dynamic cross-talk between cancer cells and cells of the TME. We envision that these methods to detect protease activity across scales could yield rich functional data about the tumor microenvironment and translate to cancer diagnostics and therapeutics.

### Results

### Profiling Protease Activity In Vivo to Monitor Tumor Progression and Treatment Response

We first sought to establish the ability of our activity-based profiling framework to noninvasively detect and monitor disease over tumor progression and treatment response. We utilized an autochthonous mouse model of ALK⁺ NSCLC as a model system, in which intrapulmonary administration of an adenovirus encoding two guide RNAs and Cas9 resulted in oncogenic rearrangement of the Eml4 and Alk genes leading to the formation of lung tumors that histologically resembled human lung adenocarcinoma [25]. Hereafter, we refer to this Eml4-Alk driven model of NSCLC as the Eml4-Alk model. We queried a bulk RNA sequencing (RNA-seq) dataset of Eml4-Alk lungs [26] and identified several proteases overexpressed in Eml4-Alk mice (FIG. 6). To noninvasively monitor protease activity in the Eml4-Alk model, we engineered a multiplexed panel of activity-based nanosensors that can be selectively activated by dysregulated proteases within the TME to release mass-barcoded peptide reporters that clear into the urine[22]. Critically, these nanosensors (Table 2) use peptide substrates that can be recognized in vitro by a range of metallo-, serine, and aspartic proteases [22] and that require substrate cleavage for activation and release of the mass-barcoded urinary reporters. As early as 3.5 weeks after tumor induction, we intratracheally administered the nanosensor panel into Eml4-Alk and healthy control mice, and observed that several nanosensors were differentially cleaved by proteases in the pulmonary microenvironment enabling differentiation of tumor-bearing and healthy mice at an early stage of tumor progression

TABLE 2
Reporter and substrate sequences for in vivo activity-based nanosensors.
Lowercase letters: d-amino acids; ANP: 3-Amino-3-(2-nitro-phenyl)propionic acid;
Cha: 3-Cyclohexylalanine; Cys(Me): methyl-cysteine

| | | Photolabile | | |
|---|---|---|---|---|
| Name | Reporter | group | Substrate | Nanocarrier |
| PP01 | e(+2G)(+6V)ndneeGFFsAr | ANP | GGPQGIWGQC | PEG-8 |
| | (SEQ ID No: 197) | | (SEQ ID No: 211) | 40 kDa |
| PP02 | eG(+6V)ndneeGF(+1F)s(+1A)r | ANP | GGPVGLIGC | PEG-8 |
| | (SEQ ID No: 198) | | (SEQ ID No: 212) | 40 kDa |
| PP03 | e(+3G)(+1V)ndneeGFFs(+4A)r | ANP | GGPVPLSLVM | PEG-8 |
| | (SEQ ID No: 199) | | (SEQ ID No: 213)C | 40 kDa |
| PP04 | e(+2G)Vndnec(+2G)FFs(+4A)r | ANP | GGPLGLRSWC | PEG-8 |
| | (SEQ ID No: 200) | | (SEQ ID No: 214) | 40 kDa |
| PP05 | eGVndnee(+3G)(+1F)Fs(+4A)r | ANP | GGPLGVRGKC | PEG-8 |
| | (SEQ ID No: 201) | | (SEQ ID No: 215) | 40 kDa |
| PP06 | e(+2G)(+6V)ndnec(+3G)(+1F) | ANP | GGfPRSGGGC | PEG-8 |
| | (+1F)s(+1A)r (SEQ ID No: 202) | | (SEQ ID No: 216) | 40 kDa |
| PP07 | eG(+6V)ndnee(+3G)(+1F)Fs | ANP | GGLGPKGQTGC | PEG-8 |
| | (+4A)r (SEQ ID No: 203) | | (SEQ ID No: 217) | 40 kDa |
| PP08 | e(+3G)(+1V)ndneeG(+10F)FsAr | ANP | GGGSGRSANAKGC | PEG-8 |
| | (SEQ ID No: 204) | | (SEQ ID No: 218) | 40 kDa |
| PP09 | eGVndneeGF(+10F)s(+4A)r | ANP | GGKPISLISSGC | PEG-8 |
| | (SEQ ID No: 205) | | (SEQ ID No: 219) | 40 kDa |
| PP10 | e(+2G)(+6V)ndneeG(+10F)(+1F) | ANP | GGILSRIVGGGC | PEG-8 |
| | s(+1A)r (SEQ ID No: 206) | | (SEQ ID No: 220) | 40 kDa |
| PP11 | e(+3G)(+1V)ndnee(+2G)(+10F) | ANP | GGSGSKIIGGGC | PEG-8 |
| | Fs(+4A)r (SEQ ID No: 207) | | (SEQ ID No: 221) | 40 kDa |
| PP12 | eGVndneeG(+10F)(+10F)sAr | ANP | GGPLGMRGGC | PEG-8 |
| | (SEQ ID No: 208) | | (SEQ ID No: 222) | 40 kDa |
| PP13 | e(+2G)(+6V)ndnee(+3G)(+10F) | ANP | GGP-(Cha)-G- | PEG-8 |
| | (+1F)s(+4A)r (SEQ ID No: 209) | | Cys(Me)-HAGC | 40 kDa |
| | | | (SEQ ID No: 223) | |
| PP14 | e(+3G)(+1V)ndnee(+2G)(+10F) | ANP | GGAPFEMSAGC | PEG-8 |
| | (+10F)sAr (SEQ ID No: 210) | | (SEQ ID No: 224) | 40 kDa |

We then assessed whether activity-based nanosensors could rapidly and quantitatively monitor the dynamics of tumor progression and regression. We treated Eml4-Alk mice with the first-line clinical ALK inhibitor alectinib [27] and monitored changes in pulmonary protease activity over a two-week treatment course that resulted in rapid and robust tumor regression (FIG. 7a-b). Strikingly, we observed that alectinib treatment significantly altered pulmonary protease activity within just 3 days of treatment initiation, with 12 of 14 reporters exhibiting differential enrichment in the urine of vehicle-versus alectinib-treated mice Signal trajectories for each of the individual nanosensors revealed distinct patterns in their dynamics. Notably, cleavage of a subset of nanosensors (e.g., PP01, PP07, PP10) increased over time in vehicle-treated mice as tumors progressed but rapidly regressed following alectinib treatment, while the cleavage of other nanosensors (e.g., PP04) transiently increased upon initiation of alectinib treatment and then returned towards baseline levels. Principal component analysis (PCA) revealed that protease activity in vehicle-treated Eml4-Alk mice grew more divergent from healthy controls over the course of tumor progression, whereas that of alectinib-treated mice became more similar to healthy controls. As such, a random forest classifier trained on urinary reporter signatures from a subset of Eml4-Alk mice achieved highly accurate classification of therapeutic response to ALK inhibition (Table 3).

TABLE 3
Composition of cohorts for random forest classification. Cohort numbers
used to train and test random forest classifiers applied Time points
designate days post-treatment initiation and weeks post-tumor induction.
Individual samples across the three time points were randomly selected
for train or test cohorts per independent classification trial, according
to a 50-50 split between train and test cohorts.

| | Eml4-Alk, Vehicle | Em14-Alk, Alectinib |
|---|---|---|
| 3 days treatment (5.5 wks) | 19 | 18 |
| 7 days treatment (6 wks) | 13 | 12 |
| 14 days treatment (7 wks) | 14 | 14 |
| Total | 46 | 44 |
| Train | 23 | 22 |
| Test | 23 | 22 |

### Multiplexed Spatial Localization of Protease Activity

We next sought to investigate the biological drivers of the observed protease activity dysregulation in Eml4-Alk mice. To this end, we reasoned that tissue-level spatial localization of protease activity against target peptide substrates could facilitate biological interpretation. For instance, understanding where in the tumor microenvironment PP01 is cleaved would point us toward proteolytically active cells that may play important roles in tumorigenesis and thus represent potential diagnostic or therapeutic targets. Because our in vivo nanosensors use peptide cleavage as their mechanism of release and measurement, we translated their substrates into in situ activatable zymography probes (AZPs) that also rely on substrate-specific proteolytic cleavage for activation [28]. Within an AZP, a protease-cleavable substrate links a fluorophore-tagged, positively-charged domain (polyR) with a negatively-charged domain; this structure remains complexed in the absence of proteolytic activation. When AZPs are applied to fresh-frozen tissue sections in a manner analogous to immunofluorescence staining, substrate cleavage by tissue-resident enzymes liberates the tagged polyR to electrostatically interact with and bind the tissue, enabling localization of protease activity by microscopy.

We thus leveraged AZPs for on-tissue spatial localization of protease activity against target peptide substrates nominated from in vivo profiling. We selected three nanosensors whose signals tracked with tumor progression and alectinib treatment response (PP01, PP07, PP10 and incorporated them into individual AZPs with orthogonal fluorophores (Z1, Z7, Z10, respectively; FIG. 2a). We applied these three AZPs to consecutive sections of lung tissue from Eml4-Alk mice 7 weeks after tumor induction and observed protease-mediated, tumor-specific labeling of Z1 and Z7, but not Z10 (FIG. 8a-c). Intriguingly, we observed that the Z1 staining pattern appeared to follow a spindle-like pattern, distinct from the more diffuse Z7 staining pattern. Thus, we sought to multiplex the three AZPs on a single slide and assess any differences in staining pattern. Once again, we detected protease-mediated, tumor-specific labeling of Z1 and Z7 (FIG. 2c-d). We also observed fluorescence signal in the Z10 (FITC) channel (FIG. 8d), which we presumed to be due to spectral overlap from the Z7 (TRITC) channel given the results of the single color stain (FIG. 8c). This multiplexed in situ labeling revealed a distinct pattern of spatial localization for Z1 relative to Z7 (FIG. 2d). Qualitatively, while Z7 exhibited broad, diffuse staining throughout Eml4-Alk tumor tissue, Z1 exhibited a prominent spindle-like pattern, suggesting different cells of origin for the proteases cleaving the two probes (FIG. 2d). Tissue labeling of both Z1 and Z7 was significantly abrogated by addition of a cocktail of protease inhibitors (P<0.0001; FIG. 2e-f).

Delineating protease class- and cell type-specific activity with AZPs Having demonstrated that orthogonal AZPs could be simultaneously multiplexed, we next endeavored to show that they could be used to identify protease families and cell compartments contributing to their cleavage. Due to its prominent in situ localization pattern and the significant in vivo correlation of PP01 with tumor progression, we nominated Z1 for further investigation and sought to understand the processes driving cleavage of this peptide (“S1” for cleavage motif; Table 4). Whereas healthy lungs exhibited undetectable Z1 staining, Eml4-Alk tumors exhibited strong, spindle-like staining that was distinct from the uniform staining pattern of a free polyR binding control (FIG. 3a, FIG. 9a-b). Furthermore, lungs from alectinib-treated Eml4-Alk mice exhibited fewer regions of Z1 staining (FIG. 9c). To further verify that the Z1 localization pattern truly reflected specific protease expression by the labeled cells, rather than nonspecific labeling (i.e., due to non-uniform distribution of charge), we precleaved Z1 in vitro with recombinant fibroblast activation protein (FAP; FIG. 10) and compared its tissue labeling to that of intact Z1 activated by tissue-resident enzymes (FIG. 11a-b). While intact Z1 maintained its spindle-like spatial pattern, precleaved Z1 exhibited diffuse, uniform labeling that mirrored that of a free polyR, verifying that probe localization depended on local in situ activation (FIG. 11a-b).

TABLE 4
Peptide sequences for activatable zymography probes.
Lowercase letters: d-amino acids; QSY21-Cy5: FRET pair, with Cy5 as fluorophore and QSY21
as quencher; PEG2K: (poly)ethylene-glycol, MW 2000 g/mol; o: 5-amino-3-oxopentanoyl;
U: succinoyl; X: 6-aminohexanoyl

| Name | Sequence | Readout |
|---|---|---|
| Z1 | UeeeeeeeeXGGPQGIWGQGrrrrrrrrrX-k(Cy5) SEQ ID NO: 168 | Fluorescence (in situ) |
| Z7 | UeeeeeeeeXGGLGPKGQTGGrrrrrrrrrX-k(Cy3) SEQ ID NO: 169 | Fluorescence (in situ) |
| Z10 | UeeeeeeeeXGILSRIVGGGrrrrrrrrrX-k(FAM) (SEQ ID NO: 170) | Fluorescence (in situ) |
| QZ1 | (QSY21)-eeeeeeeee-c(PEG2K)-oGGPQGIWGQG-rrrrrrrrr-k(Cy5) | Fluorescence (in vitro/ |
| | (SEQ ID NO: 167) | in vivo) |
| polyR | rrrrrrrrrX-k(Cy7) (SEQ ID No: 225) | Fluorescence (in situ) |
| S1 | GGPQGIWGQC (SEQ ID No: 226) | Cleavage motif |

To determine class-specific contributions to its cleavage, we applied Z1, whose substrate can be recognized by both matrix metalloproteinases (MMPs) and serine proteases [15, 29, 22], to Eml4-Alk lung tissue sections in the absence of protease inhibitors, with a broad-spectrum cocktail of protease inhibitors, with the MMP inhibitor marimastat, or with the serine protease inhibitor 4-(2-aminoethyl)benzenesulfonyl fluoride hydrochloride (AEBSF; FIG. 3b). As expected, incubation with broad-spectrum protease inhibitors significantly abrogated Z1 labeling (P<0.0001; FIG. 3c). Qualitatively, Z1 signal was largely preserved in sections incubated with marimastat (FIG. 3b). While marimastat did reduce Z1 signal, it remained significantly increased relative to the broad-spectrum inhibitor condition (P<0.0001; FIG. 3c). In contrast, incubation with AEBSF completely abrogated Z1 tissue labeling to the level of broad-spectrum inhibition (P<0.0001 uninhibited vs. AEBSF, P=0.7127 broad-spectrum inhibition vs. AEBSF; FIG. 3c), suggesting that serine proteases are primarily responsible for cleaving Z1 in Eml4-Alk tumors.

Though Eml4-Alk tumors are adenocarcinomas and thus consist primarily of epithelial cells, the distinct spindle-like labeling pattern of Z1 raised the possibility that Z1 might be cleaved by proteases expressed by non-epithelial cells of the TME. To this end, we applied Z1 to Eml4-Alk lung tissue sections and simultaneously stained for both E-cadherin, an epithelial cell marker, and vimentin, the intermediate filament of mesenchymal cells (FIG. 3d-e, FIG. 12). We observed an overlap between Z1 and vimentin in Eml4-Alk tissue (FIG. 3e), confirmed by staining for the aforementioned two markers alone (FIG. 13a). In contrast, Eml4-Alk tumor regions exhibited distinct localization of Z1 relative to epithelial cells marked by E-cadherin (FIG. 13b), while there was diminished Z1 labeling in healthy lungs altogether (FIG. 13c-d). Quantitatively, vimentin-positive spindle-like cells exhibited increased Z1 intensity relative to E-cadherin-positive cells or vimentin-positive rounded cells (likely tumor-associated macrophages) (FIG. 3f, P<0.0001; see Methods for details). These results suggest that vimentin-positive, spindle-like cells are associated with the serine protease activity cleaving Z1 and, more broadly, that AZPs can delineate class-specific and cell type-associated activity patterns.

### Multimodal Spatial Profiling to Functionally Query the TME

Next, we sought to further investigate the cell type(s) responsible for Z1 cleavage, as their protease activity suggested aberration and potential contribution to tumor progression. The distinct spatial pattern of Z1 staining led us to hypothesize that this probe could be labeling cells of the tumor vasculature, rather than cells of immune or other mesenchymal compartments. We thus applied Z1 to Eml4-Alk and healthy lungs and co-stained for the endothelial cell marker CD31 (PECAM-1; FIG. 14a). Qualitatively, while both Eml4-Alk and healthy lungs exhibited an abundance of endothelial cells as evidenced by CD31-positivity, Z1 labeling was enriched in Eml4-Alk tumors relative to healthy lungs and tended to colocalize with CD31-positive cells (FIG. 4a). Cell-by-cell quantification of Z1 and CD31 staining intensities across entire lung tissue sections identified a strong positive correlation in Eml4-Alk tissue (R²=0.67; FIG. 14b). Indeed, Z1 staining was significantly increased in CD31-high cells in Eml4-Alk lung tissue sections relative to CD31-high cells in healthy lungs, as well as relative to CD31-low cells in both Eml4-Alk and healthy tissue (P<0.0001; FIG. 4b), suggesting specific activity associated with the Eml4-Alk tumor endothelium. Furthermore, immunostaining for VE-cadherin, a strictly endothelial-specific adhesion molecule, revealed a spindle-like pattern of expression within Eml4-Alk tumors that mimicked Z1 staining (FIG. 15).

In addition to endothelial cells, the vasculature also contains contractile vascular smooth muscle cells that line the vessel walls. Capillaries and microvessels, such as those within the lungs, contain a mural, periendothelial mesenchymal cell population known as pericytes (FIG. 4c), which help regulate vascular function and can be actively recruited into the vasculature during angiogenesis. Eml4-Alk tumors stained positively for α-smooth muscle actin (αSMA), a canonical vascular smooth muscle cell marker that can be expressed by tumor pericytes but is often absent in quiescent pericytes in normal tissues. Indeed, normal adjacent tissue (NAT) showed reduced αSMA expression. To further corroborate the likely presence of pericytes within the tumor vasculature, we stained Eml4-Alk and healthy lungs for CD31 and a second pericyte marker, the muscular intermediate filament desmin, and observed desmin-positive cells surrounding CD31-positive endothelial cells within Eml4-Alk tumors but not in NAT nor healthy tissue. Finally, we stained Eml4-Alk and healthy lung tissue sections for the pericyte-associated marker PDGFRβ. The PDGF-B/PDGFRβ signaling pathway is a key axis of interaction between endothelial cells and pericytes, wherein PDGF-B released from angiogenic endothelial cells binds to PDGFRβ on the surface of pericytes, facilitating their recruitment. Eml4-Alk tumors stained positively for both CD31 and PDGFRβ, while NAT from Eml4-Alk lungs (FIG. 4d) and healthy tissue from healthy lungs did not express PDGFRβ despite abundant CD31 expression. Within the tumor vasculature specifically, PDGFRβ-positive cells wrapped around CD31-positive cells, consistent with the expected localization and function of pericytes (FIG. 4d).

To assess its localization with respect to cells of the tumor vasculature, we applied Z1 to Eml4-Alk lung tissue sections with concurrent staining for both the endothelial marker VE-cadherin and the pericyte marker desmin. We observed robust Z1 labeling together with VE-cadherin and desmin expression within Eml4-Alk tumors (FIG. 4e). However, NAT displayed decreased Z1 and desmin staining despite maintaining VE-cadherin positivity. Closer inspection of Z1 labeling within Eml4-Alk tumors revealed an association between all three stains (FIG. 4f). Colocalization analysis demonstrated a correlation between desmin and VE-cadherin staining, consistent with the close proximity of both cell types within capillaries, and additionally showed that both desmin and VE-cadherin correlated with Z1 labeling (FIG. 4g). Through coupling of AZPs with other spatial measurements, these results suggest that the sensor S1 reads out specific functional (i.e., serine protease activity) and compositional (i.e., increased pericyte coverage) changes within the Eml4-Alk TME.

Complementing Protease Activity Measurements with Single-Cell Transcriptomics to Characterize the Eml4-Alk TME

We next sought to further characterize the phenotypes of the identified S1-associated, tumor vasculature cell populations in order to understand potential mechanisms for the dysregulation of these cells. To complement our in vivo and in situ activity measurements, we performed single-cell RNA sequencing (scRNA-seq) to obtain an unbiased view of the cellular and transcriptomic landscape of Eml4-Alk lungs. Graph-based clustering of uniform manifold approximation and projection (UMAP) captured the transcriptomic landscape of Em14-Alk lungs, where we annotated eight significant groups of cell types based on expression of previously reported marker genes (Table 5). Given that S cleavage in situ localized to cells of the tumor vasculature, we defined marker gene modules for both endothelial and pericyte populations and L computed their expression scores across all cells in Eml4-Alk lungs. The identified population of endothelial cells expressed several markers within a module of 28 genes canonically associated with angiogenesis. Marker gene analysis additionally revealed a small population of pericytes within the larger stromal cluster.

TABLE 5
Marker genes used to annotate cell types in scRNA-
seq data from Eml4-Alk and healthy lungs.

| Annotated cell type label | Marker genes |
|---|---|
| Capillary endothelium | Cd36, Cd93, Gpihbp1, Plvap, Ptprb |
| (general) | |
| Capillary endothelium | Car4, Ednrb, Fibin, Prx |
| (aerocytes) | |
| Venous endothelium | Amigo2, Slc6a2, Vegfc |
| Lymphatic endothelium | Mmrn1, Pdpn, Pecam1, Prox1, Thy1 |
| Alveolar epithelium: | Annotated based on AT1 & AT2 |
| AT1 & AT2 | marker genes |
| Alveolar type 1 | Ager, Cdh1, Epcam, Hopx, Pdpn |
| Alveolar type 2 | Aqp5, Cdh1, Epcam, Etv5, Muc1, |
| | Nkx2-1, Sftpb, Sftpc |
| Club cells | Cckar, Cdh1, Epcam, Scgb1a1, |
| | Scgb3a2 |
| Ciliated epithelium | Ccdc78, Cdh1, Epcam, Fam 183b, |
| | Foxj1 |
| Stroma | Annotated based on below marker |
| | genes |
| Pericyte | Acta2, Cspg4, Des, Higd1b, Nes, |
| | Pdgfrb, Trpc6 |
| Adventitial fibroblast | Entpd2, Pi16, Serpinf1 |
| Alveolar fibroblast | Fgfr4, Slc7a10, Slc38a5 |
| Matrix fibroblast | Colla2, Colla1, Fn1, Tcf21 |
| Myofibroblast | Aspn, Fgf18, Pdgfra, Rgs2, Wif1 |
| Mesothelial | Msln, Upk3b, Wt1 |
| Smooth muscle | Acta2, Cnn1, Tagln |

Spatial profiling had indicated the presence of cells positive for each of αSMA, desmin, and PDGFRβ within Em14-Alk tumors but not within NAT or healthy lung tissue (FIG. 4c), raising the question of whether pericytes were specifically recruited into the TUE. We queried scRNA-seq data from healthy mouse lungs for the pericyte marker gene module and identified a small population of cells exhibiting this signature, in line with pericyte identification reported in previous transcriptomic cell atlas studies in human [35] and mouse [34] lungs. PDGF signaling has been shown to facilitate recruitment of pericytes into the tumor vasculature as a means to stabilize vessels and promote the establishment of an angiogenic, reactive TME. We therefore queried expression of both PDGF ligands (Pdgfa, Pdgfb) and receptors (Pdgfra, Pdgfrb) in the Eml4-Alk scRNA-seq dataset and found that expression of Pdgfra and Pdgfrb was exclusive to the stromal cluster. This analysis also revealed robust and specific expression of Pdgfb in endothelial cell populations. Visualization via UMAP corroborated that expression levels of Pdgfb and Pdgfrb matched the distributions of endothelial cell and pericyte populations, respectively.

These results raised the possibility of paracrine PDGF signaling between endothelial cells and PDGFR-positive stromal cells in Eml4-Alk tumors. To investigate whether this axis was transcriptionally dysregulated, we conducted an integrative analysis of scRNA-seq data from Eml4-Alk and healthy lungs. Differential gene expression analysis across this integrated dataset showed that Pdgfb was overexpressed in cells from both capillary endothelial cell compartments in the TME relative to healthy lungs (P_{adj}<0.0001; Table 6). However, expression of the PDGF receptors Pdgfra and Pdgfrb remained consistent between the total stromal populations from both conditions (Table 6).

TABLE 6
PDGF signaling genes are differentially expressed in scRNA-seq data from Eml4-Alk and healthy lungs.
Differential gene expression analysis was conducted on scRNA-seq data from cells within each of the
indicated cell compartments, and results were filtered for genes from the PDGF signaling axis. Significance
was calculated by the Wilcoxon rank-sum two-sided test with Benjamini-Hochberg correction.

| | Cap. endothelium (general) | | Stroma | | Cap. endothelium (aerocyte) | | Ciliated epithelium | |
|---|---|---|---|---|---|---|---|---|
| Gene | log₂(EA/WT) | P_{adj} | log₂(EA/WT) | P_{adj} | log₂(EA/WT) | P_{adj} | log₂(EA/WT) | P_{adj} |
| Pdgfa | 0.578 | 8.29 × 10⁻¹ | 0.07840 | 1.00 | 1.670 | 3.85 × 10⁻¹ | 0.999 | 1.00 |
| Pdgfb | 0.676 | 1.39 × 10⁻²⁵ | 0.824 | 1.00 | 0.835 | 1.77 × 10⁻¹¹ | 10.331 | 1.00 |
| Pdgfra | −0.396 | 1.00 | −0.0295 | 1.00 | −0.145 | 1.00 | 11.335 | 1.00 |
| Pdgfrb | −1.776 | 1.00 | 0.178 | 1.00 | −7.183 | 1.00 | 0.000 | 1.00 |
| Cxcl12 | 1.453 | 1.73 × 10⁻¹⁹⁵ | 1.542 | 5.77 × 10⁻¹⁹ | 1.537 | 1.32 × 10⁻² | 11.335 | 1.00 |

These observations motivated the hypothesis that altered ligand expression by endothelial cells in the Eml4-Alk TME could be implicated in the association of pericytes to the tumor vasculature. In addition to Pdgfb, the chemokine Cxcl12, shown to play functional roles in angiogenesis and vascular recruitment of stromal cells, was robustly expressed in endothelial cells from Eml4-Alk lungs. Endothelial cells from Eml4-Alk and healthy lungs exhibited differential transcriptional landscapes, with Cxcl12 expression significantly increased in endothelial cells from Eml4-Alk lungs relative to those from healthy controls (log₂ FC=1.453. P_{adj}<0.0001; Table 6). Intriguingly, previous reports have shown that overexpression of PDGF-B can increase tumor pericyte content via induction of CXCL12 expression by endothelial cells within the TME [38].

Finally, the rapid and profound reduction in PP01 signal (in vivo) and Z1 staining (in situ) after treatment with alectinib, which theoretically should only induce apoptosis in ALK+ cancer cells, led us to investigate the role that Alk-mutant tumor cells themselves play in regulating the angiogenic tumor microenvironment. To this end, we established tumor organoids in vitro by inducing Eml4-Alk fusions in alveolar type 2 (AT2) organoids via CRISPR-Cas9 [39]. Transcriptomic analysis of Eml4-Alk-mutant organoids revealed enrichment of genes associated with angiogenesis, including Pdgfb, suggesting that Alk-mutant tumor cells themselves may contribute directly to endothelial cell and pericyte recruitment. These results also suggest a potential mechanism by which alectinib treatment may indirectly impact the tumor vasculature and its associated protease activity.

### Activity-Based Cell Sorting Enables Multimodal Phenotypic Characterization of Eml4-Alk Lung Cancer

Our results thus far suggested that alectinib, a therapy targeted toward ALK-positive tumor cells, induced rapid and dramatic changes in the proteolytic activity of presumably ALK-negative vascular cells within the tumor microenvironment. Follow-up transcriptomic profiling unearthed a potential mechanism of communication between ALK+ tumor cells, endothelial cells, and pericytes mediated by PDGF and CXCL12. However, as the protease profiling and transcriptomic methods were decoupled, it is impossible to prove that the cells analyzed in the transcriptomic experiments were equivalent to the proteolytically active cells identified in our in situ experiments. We therefore sought to establish a method to isolate individual cells on the basis of their protease activity. We hypothesized that AZPs containing fluorophore-quencher pairs could function as activatable cellular tags in vivo to label cells with membrane-bound or proximal protease activity, such that tagged cells could be subsequently sorted via flow cytometry (FIG. 5a). In this design, following systemic administration, degradation of the protease-cleavable linker activates fluorescence and liberates the fluorophore-tagged polyR such that it can bind and tag nearby cells, functioning analogously to a cell penetrating peptide. Thus, we reasoned that probe labeling after proteolytic activation (e.g., by cell-surface or proximal proteases) would facilitate isolation of tagged cells via fluorescence-activated cell sorting (FACS) and enable subsequent downstream phenotypic characterization (FIG. 5a).

We applied this activity-based cell sorting assay to directly isolate and then phenotypically characterize the Eml4-Alk cell compartment associated with S1 cleavage (FIG. 5a). We designed a fluorescent quenched probe, QZ1, that incorporated S1 as a protease-cleavable linker. Cy5-labeled QZ1 was PEGylated to improve stability and drive tissue accumulation [40, 28], and administered intravenously into age-matched Eml4-Alk and healthy mice. Eml4-Alk mice were assessed at 12 weeks post tumor induction, at which point the lungs contain multiple lung adenocarcinoma lesions [25]. Two hours post-injection, significantly increased Cy5 fluorescence was found in explanted Eml4-Alk lungs relative to healthy lungs (P<0.0001; FIG. 5b-c), enabling perfect discrimination of Eml4-Alk and healthy mice (AUC=1.000; FIG. 5d).

Following imaging, single-cell suspensions were prepared from dissociated Eml4-Alk lungs, and fluorescence-activated cell sorting (FACS) was used to sort all live, non-hematopoietic nucleated cells by QZ1 signal, demonstrating the feasibility of the activity-based cell sorting method (FIG. 5a). We also performed co-immunostaining for markers associated with mesenchymal cell lineages. Intriguingly, whereas Eml4-Alk lungs exhibited increased QZ1 staining across cells positive for the mesenchymal cell markers (FIG. 16), healthy lungs exhibited a more variable pattern in a separate experiment (FIG. 17). Bulk RNA-seq on sorted QZ1-high (QZ1-hi) and QZ1-low (QZ1-lo) populations from Eml4-Alk lungs was used to characterize gene expression differences between the two compartments (FIG. 5e). Several canonical markers of endothelial cells (Cdh5, Eng, Vwf, Pecam1), pericytes (Cd248, Pdgfrb, Des), as well as vascularization and angiogenesis were among the most upregulated genes in the QZ1-hi population (FIG. 5f). Gene set enrichment analysis corroborated that the dominant cell types associated with QZ1 labeling were endothelial and mesenchymal cell types, including pericytes, while gene sets associated with epithelial cells were significantly downregulated. Cxcl12 and Pdgfrb were overexpressed in the QZ1-hi compartment, as were markers of additional angiogenesis signaling axes, including the VEGF (Flt1/4), Notch (Notch1/4, Dll1/4), and Tie (Tek, Angpt1/2) pathways (FIG. 5f). Indeed, the QZ1-hi compartment was significantly enriched for functional modules associated with vasculogenesis, vascular development, endothelial cell migration, and mesenchymal recruitment (FIG. 5g). These results provide further evidence that S1 is cleaved by proteases associated with the angiogenic tumor vasculature. More broadly, they validate that activity-based cell sorting can be used to isolate cells on the basis of their protease activity, enabling deep phenotypic characterization.

### Discussion

Our results establish a workflow for profiling protease activity across multiple scales—at the organism, tissue, and cellular levels- and demonstrate the utility of our methods for noninvasive monitoring and functional characterization of tumor progression and treatment response, showcased in the context of Eml4-Alk mutant lung cancer treated with targeted therapy (FIG. 1). We first demonstrated that multiplexed panels of protease-responsive nanosensors can quantitatively track disease dynamics in vivo to yield activity-based biomarkers of tumor progression and targeted therapy response. We directly translated substrates nominated from in view) profiling into in situ protease activity probes (AZPs) and thus identified a tumor-specific serine protease activity signal that increased with tumor progression, rapidly decayed after therapy, and localized specifically to the pericyte-invested tumor vasculature. We complemented our activity measurements with single-cell transcriptomic analysis, which identified overexpression of paracrine signaling factors in endothelial cells from tumor-bearing lungs and suggested a possible mechanism for endothelial cell-pericyte cross-talk within the TME. Finally, we designed a high-throughput method to isolate cells based on their protease activity and leveraged it to discover a population of proteolytically active, vasculature-associated cells harboring pro-angiogenic transcriptional programs. Together, these methods revealed that the functionally aberrant tumor vasculature rapidly responds to tumor cell-targeted inhibition of oncogenic signaling and demonstrated that protease activity serves as an informative proxy for this process.

Our work establishes a multiplexed in situ activity assay that enables direct on-tissue comparison of spatial localization patterns of distinct proteases. We also demonstrate that AZPs can be used to delineate protease class-specific activity signatures through targeted inhibitor ablations in situ. Given that our results indicate that serine proteases cleave S1 in the Eml4-Alk model (FIG. 3c), additional molecular profiling will be necessary to identify which protease(s) are responsible. The angiogenesis-associated proteases Plat (tPA) and Dpp4 (DPP4), as well as the membrane serine protease Fap (FAP or seprase), which can be selectively expressed by tumor pericytes [42, 43], were amongst the serine proteases overexpressed in the QZ1+ population. In vitro screening, targeted small molecule inhibition, or specific knockout of individual enzyme targets could help identify the serine protease(s) that cleave S1 in the Eml4-Alk model. Parallel functional studies, in particular in tumor-derived organoids or vascularized co-culture systems, may help determine the specific contributions of candidate serine proteases to vascular remodeling and angiogenesis in ALK⁺ lung cancer.

This work establishes AZPs as an activity-based cellular tag for sorting individual cells based on endogenous protease activity. Administration of AZPs in vivo, followed by tissue dissociation and FACS, enabled isolation of cells exhibiting a specific pattern of protease activity. By coupling this assay to immunostaining and RNA-seq, we demonstrate that activity-based cell sorting can enable multimodal characterization across the activity, protein, and gene expression levels. Probes similar in concept to AZPs could extend activity-based cell sorting to other classes of enzymes. In addition, integrating activity-based cell sorting with large-scale omics measurements and machine learning could inspire single-cell multiomics that ends at the level of actuated biological function. We envision that the ability to sort cells by enzymatic activity could yield insights into enzymatic dysregulation in disease, enable multimodal approaches to characterize biological systems, and inform diagnostic and therapeutic interventions.

Our results demonstrate that protease activity directly complements measurements of protein and transcript abundance, and that this multimodal profiling enables discovery-based functional characterization of the TME. By applying our activity-based profiling methods to the Eml4-Alk model of NSCLC, we discovered aberrant serine protease activity that is specific to the tumor vasculature and rapidly responds to inhibition of an adjacent cancer-cell specific pathway. Through a combination of spatial profiling and scRNA-seq analysis, we found evidence suggestive of increased pericyte coverage within the Eml4-Alk tumor vasculature, potentially mediated by altered paracrine signaling via PDGF and CXCL12. PDGF-expressing endothelial cells can produce CXCL12, a chemokine shown to facilitate recruitment of stromal cells and to promote angiogenesis. This represents one altered function of the Eml4-Alk vasculature, whose dysregulation and angiogenic phenotype can be read out by altered protease activity measured by the protease sensor S1. Additional investigation into CXCL12 induction and its downstream effects will help determine whether or not chemokine production causally depends on the protease activity, or if it is a parallel altered function of the dysregulated tumor vasculature.

Though mechanistic experiments will be necessary to ascertain whether pericytes are actively recruited into the TME, our findings raise the possibility that S1 cleavage, which is elevated within Eml4-Alk tumors and localizes specifically to the vasculature, could be a result of the coordinated action of intratumoral pericytes and endothelial cells associated with neoangiogenic vessels. Necessary future work to establish tractable ex vivo models, such as vascularized Eml4-Alk tumor-derived organoids or co-culture systems, will in turn enable such functional studies to identify and validate mechanistic targets. Our finding that the functionally aberrant tumor vasculature rapidly responds to targeted therapy motivates exploration of whether anti-angiogenic drugs, which have been clinically approved in combination with cytotoxic chemotherapy or immunotherapy [44, 45, 46], could have additive benefits when combined with molecularly targeted therapeutics like alectinib. Our study in the Eml4-Alk model serves as an example for how our activity profiling methods can be leveraged to spawn and advance hypotheses about the complex crosstalk between cancer and non-cancer cells, though complementary mechanistic and functional work is necessary to fully validate these hypotheses and establish causal mechanisms.

Finally, the activity-based profiling methods presented here could have utility in precision medicine applications. Precision cancer medicine requires granular information that cannot be accessed by traditional noninvasive imaging approaches, necessitating serial biopsies that carry significant risks and sample only a small fraction of the disease site. The ability to gain high-dimensional biological insight into a disease state with a completely noninvasive test would present an advance towards functional precision medicine. Here, we establish the capacity of noninvasive, multiplexed protease activity nanosensors to query the function and activity of specific intratumoral cell subsets over the course of tumor progression and in response to therapy. Given the modularity of this approach, high-throughput screening and generative machine learning methods could optimize activity sensors to target orthogonal axes of cancer biology. For instance, activity sensors that detect angiogenesis could be administered in combination with probe sets that read out immune invasion or metastasis risk. As a complement to this noninvasive test, a targeted panel of in situ AZPs could be used to molecularly profile individual patient biopsies for indication of signaling pathways or processes active in a patient's specific tumor. Protease activity sensors can empower patients and physicians with real-time, high-quality information to personalize treatment decisions, such as rapid prediction of immunotherapy efficacy, surveillance for recurrence after targeted therapy, or discrimination of aggressive versus indolent disease.

In summary, we present an integrated suite of protease activity-profiling methods that form a direct link between noninvasive enzyme sensors, high-resolution spatial profiling, and high-throughput, single-cell analytical methods like flow cytometry and RNA-seq. The modular methods described here can be readily generalized to other cancer types and hold promise for both fundamental biological investigation and translational research. We envision that these methods for profiling protease activity will help facilitate functional characterization of cancer for medical and discovery applications alike.

### Methods

### Eml4-Alk Mouse Model of Non-Small-Cell Lung Cancer

All animal studies were approved by the Massachusetts Institute of Technology (MIT) committee on animal care and were conducted in compliance with institutional and national policies. Reporting was in compliance with Animal Research: Reporting In Vivo Experiments (ARRIVE) guidelines. Tumors were initiated in 6-10 week old female C57BL/6J mice (Jackson Labs) by intratracheal administration of 50 μL adenovirus expressing the Ad-EA vector (VQAd Cas9 ALK EML4072415; Viraquest™; 1.5*10⁸ PFU in Opti-MEM™ with 10 mM CaCl₂). These mice are referred throughout the manuscript as “Eml4-Alk” mice. Criteria for euthanasia, as dictated by the MIT Committee on Animal Care, was body weight loss of greater than 10%, significant dyspnea, or poor body condition. Animals were monitored daily throughout all studies, and the criteria for euthanasia were not met. Healthy control cohorts consisted of age- and sex-matched mice (i.e., female C57BL/6J, Jackson Labs) that did not undergo intratracheal administration of Ad-EA adenovirus.

### Alectinib Treatment

Eml4-Alk mice were randomized to receive either control vehicle or alectinib (MedChemExpress), at 20 mg/kg prepared directly in drug vehicle, daily by oral gavage for 14 consecutive days. Drug vehicle consisted of: 10% (v/v) dimethylsulfoxide (DMSO; Sigma Aldrich), 10% (v/v) Cremophor™ EL (Sigma Aldrich), 15% (v,v) poly(ethylene glycol)-400 (PEG400; Sigma Aldrich), 15% (w/v) (2-Hydroxypropyl)-β-cyclodextrin; Sigma Aldrich). Mice were monitored daily for weight loss and clinical signs. Investigators were not blind with respect to treatment.

### In Vivo Characterization of Activity-Based Nanosensors

All activity-based nanosensor experiments were performed in accordance with institutional guidelines. Tumor-bearing mice and age-matched controls were administered activity-based nanosensor constructs via intratracheal intubation at 3.5, 5, 5.5, 6, and 7 weeks after tumor induction, with treatment of vehicle control or alectinib beginning at 5 weeks after tumor induction in Eml4-Alk mice and continuing for 2 weeks. Nanosensors for urinary experiments were synthesized by CPC Scientific. The urinary reporter glutamate-fibrinopeptide B (Glu-Fib) was mass barcoded for detection by mass spectrometry. Sequences are provided in Table 2. Nanosensors were dosed (50 μL total volume, 20 μM each nanosensor) in mannitol buffer (0.28 M mannitol, 5 mM sodium phosphate monobasic, 15 mM sodium phosphate dibasic, pH 7.0-7.5) by intratracheal intubation. Anesthesia was induced by isoflurane inhalation, and mice were monitored during recovery. For intratracheal instillation, a volume of 50 μL was administered by passive inhalation following intratracheal intubation with a 22G flexible plastic catheter (Exel). Intratracheal instillation was immediately followed by a subcutaneous injection of PBS (200 μL) to increase urine production. Bladders were voided 60 minutes after nanosensor administration, and all urine produced 60-120 min after administration was collected using custom tubes in which the animals rest upon 96-well plates that capture urine. Urine was pooled and frozen at −80° C. until analysis by LC-MS/MS.

### LC-MS/MS Reporter Quantification

LC-MS/MS was performed by Syneos Health using a Sciex™ 6500 triple quadrupole instrument. Briefly, urine samples were treated with ultraviolet irradiation to photocleave the 3-Amino-3-(2-nitro-phenyl)propionic Acid (ANP) linker and liberate the Glu-Fib reporter from residual peptide fragments. Samples were extracted by solid-phase extraction and analyzed by multiple reaction monitoring by LC-MSMS to quantify concentration of each Glu-Fib mass variant. Analyte quantities were normalized to a spiked-in internal standard and concentrations were calculated from a standard curve using peak area ratio (PAR) to the internal standard. Mean scaling was performed on PAR values to account for mouse-to-mouse differences in activity-based nanosensor inhalation efficiency and urine concentration. tatistical and machine learning analysis of urinary reporter data

Analyses of urinary reporter data were conducted using the analytic pipelines of the protease activity analysis (PAA) package [52], a publicly available Python package designed to process and visualize enzymatic activity datasets. For all urine experiments, PAR values were normalized to nanosensor stock concentrations and then mean-scaled across all reporters in a given urine sample prior to further statistical analysis. To identify differential urinary reporters, reporters were subjected to unpaired two tailed t-test followed by correction for multiple hypotheses using the Holm-Sidak method. P_{adj}<0.05 was considered significant. For treatment response classification based on urinary activity-based nanosensor signatures, randomly assigned sets of paired data samples consisting of features (the mean scaled PAR values) and labels (for example, EA, Alectinib) were used to train random forest classifiers with 100 trees. Estimates of out-of-bag error were used for cross-validation, and trained classifiers were tested on randomly assigned, held-out, independent test cohorts. Ten independent train-test trials were run for each classification problem, and classification performance was evaluated with ROC statistics. Classifier performance was reported as the mean accuracy and AUC across the ten independent trials.

### AZP Peptide Synthesis and Sequences

All AZPs were synthesized by CPC Scientific (Sunnyvale, CA) and reconstituted in dimethylformamide (DMF) unless otherwise specified. AZP sequences are provided in Table 4.

In Situ Zymography with Activatable Zymography Probes

Mice were euthanized by isoflurane overdose. Lungs were then filled with undiluted optimal-cutting-temperature (OCT) compound through catheterization of the trachea; the trachea was subsequently clamped; and lungs were extracted. Individual lobes were dissected and then immediately embedded and frozen in optimal-cutting-temperature (OCT) compound (Sakura).

Cryosectioning was performed at the Koch Institute Histology Core. Prior to staining, slides were air dried, fixed in ice-cold acetone for 10 minutes, and then air dried. After hydration in PBS (3×5 minutes), tissue sections were blocked in protease assay buffer (50 mM Tris, 300 mM NaCl, 10 mM CaCl₂), 2 mM ZnCl₂, 0.02% (v/v) Brij-35, 1% (w/v) BSA, pH 7.5) for 30 minutes at room temperature. Blocking buffer was aspirated, and solution containing fluorescently labeled AZPs (1 μM each AZP) and a free poly-arginine control (polyR, 0.1 μM) diluted in the protease assay buffer was applied. Slides were incubated in a humidified chamber at 37° C. for 4 hours. For inhibited controls, 400 μM AEBSF (Sigma Aldrich), 1 mM marimastat (Sigma Aldrich), or protease inhibitor cocktail (P8340, Sigma Aldrich) spiked with AEBSF and marimastat was added to the buffer at both the blocking and cleavage assay steps. For uninhibited conditions, dimethyl sulfoxide (DMSO) was added to the assay buffer to a final concentration of 3% (v/v). For co-staining experiments, primary antibodies (E-cadherin, AF748, R&D Systems, 4 μg/mL; vimentin, ab92547, Abcam, 0.5 μg/mL; CD31, AF3628, R&D Systems, 10 μg/mL; desmin, ab227651, Abcam, 1.32 μg/mL) were included in the AZP solution. Following AZP incubation, slides were washed in PBS (3×5 minutes), stained with Hoechst (5 μg/mL, Invitrogen) and the appropriate secondary antibody if relevant (Invitrogen, 1:500), washed in PBS (3×5 minutes), and mounted with ProLong™ Diamond Antifade Mountant (Invitrogen). Slides were scanned on a Pannoramic 250 Flash III whole slide scanner (3DHistech).

### AZP Pre-Cleavage Characterization

The Z1 AZP (10 μmol/L) was incubated with recombinant fibroblast activation protein (FAP) in FAP assay buffer (50 mM Tris, 1 M NaCl, pH 7.5) overnight at 37° C. to run the cleavage reaction to completion. After precleavage with recombinant FAP, the AZP solution was diluted to a final peptide concentration of 0.1 μM in protease assay buffer. Cognate intact Z1 AZP (1 μmol/L) and precleaved Z1 AZP, each with a free polyR control (0.1 μM), were applied to fresh-frozen Eml4-Alk lung tissue sections (slide preparation described above) and incubated at 37° C. for 4 hours. After AZP incubation, slides were washed, stained with Hoechst, mounted, and scanned.

### Immunohistochemistry and Immunofluorescence Staining

Lungs were excised and either embedded in OCT, as previously described, or fixed in 10% (v/v) formalin and embedded in paraffin. Prior to staining, slides with formalin-fixed, paraffin-embedded sections were subject to deparaffinization and antigen retrieval. Prior to staining, slides with fresh-frozen sections were air dried, fixed in ice-cold acetone for 10 minutes, air dried, and re-hydrated in PBS. Sections were stained with IgG isotype controls (ThermoFisher) and primary antibodies against vimentin (ab92547, Abcam, 1.0 μg/mL), E-cadherin (AF748, R&D Systems, 4.0 μg/mL), α-SMA (ab124964, Abcam, 1.5 μg/mL), CD31 (AF3628, R&D Systems, 10 μg/mL), VE-cadherin (36-1900, Invitrogen, 10 μg/mL), PDGFRβ (3169, Cell Signaling, 1:100), and desmin (ab227651, Abcam, 1.32 μg/mL), as appropriate. For immunohistochemistry with α-SMA, slides were incubated with Rabbit-on-Rodent HRP-Polymer (RMR622, Biocare Medical) at native concentration for 30 minutes. For immunofluorescence, slides were washed in PBS, incubated with the appropriate secondary antibody (Invitrogen, 1:500) and Hoechst (5 μg/mL, Invitrogen) for 30 minutes at room temperature, and washed in PBS. Slides were scanned as previously described.

### Quantification of AZP and Immunofluorescence Staining

AZP and immunofluorescence staining was quantified in QuPath™ 0.2.3[53] and in ImageJ™ (NIH, v1.53). To perform cell-by-cell analysis, cell segmentation was performed using automated cell detection on the DAPI (nuclear) channel. For quantification of activity inhibition, AZP staining was calculated as a fold change of the mean nuclear AZP signal over the mean nuclear polyR signal. All nuclei within an individual tumor were averaged across that given tumor. Nuclei with a polyR intensity of less than 3 were excluded from analysis. For quantification of AZP intensity based on cell morphology and marker expression, cells were annotated as “vimentin-positive, spindle” if they were spindle-shaped and expressed vimentin; “E-cadherin-positive, cuboidal” if they were cuboidal-shaped and expressed E-cadherin; “vimentin-positive, round” if they were rounded and expressed vimentin. A random forest classifier was trained on all annotated cells (at least 20 cells per class) using multiple cellular features, including nuclear area and eccentricity, and mean cellular fluorescence intensity across all channels. The trained classifier was then applied to all cells across all tumors in the tissue section, and mean cellular fluorescence intensity was quantified. To assess relationship between Z1 and CD31, cell segmentation was performed as described above and correlation was assessed between mean cellular Cy5 (Z1) intensity and mean cellular FITC (CD31) intensity. Density plots were generated using the dscatter function in MATLAB (R2019b). For quantification of co-localization, JACoP™ (Just Another Co-localization Plug-in) [54] was used to determine pixel intensity-based correlations. Tumors were selected as regions of interest, and thresholds were chosen automatically using the Costes' method. Co-localization was assessed via the pairwise correlation of pixel intensities within each tumor region of interest.

### In Vivo Administration of QZ1

QZ1 (Table 4) was reconstituted to 1 mg/mL in water, then reacted with mPEG-Maleimide, MW 2000 g/mol (Laysan Bio), for PEG coupling via maleimide-thiol chemistry. After completion of the reaction, the final compound was purified using HPLC. All reactions were monitored using HPLC connected with mass spectrometry. Characterization of the final compound, QZ1-(PEG2K), using HPLC and MALDI-MS indicated that products were obtained with more than 90% purity and at the expected molecular weight. Eml4-Alk mice (11-12 weeks post tumor induction) and age- and sex-matched C57BL/6J healthy controls (Jackson Labs; 18-22 weeks) were anesthetized using isoflurane inhalation (Zoetis). QZ1-(PEG2K) (4.5 nmoles in 0.9% NaCl) was administered intravenously via tail vein injection. Two hours after probe injection, mice were imaged on an in vivo imaging system (IVIS, PerkinElmer) by exciting Cy5 at 640 nm and measuring emission at 680 nm. Mice were subsequently euthanized by isoflurane overdose followed by cervical dislocation. Lungs were dissected and explanted for imaging via IVIS. Fluorescence signal intensity was quantified using the Living Image software (PerkinElmer, v4).

### Preparation of Single-Cell Suspensions

Eml4-Alk mice (10-12 weeks post tumor induction) and age- and sex-matched C57BL/6J healthy controls (Jackson Labs; 18-22 weeks) were euthanized by isoflurane overdose, and lungs were excised, separated into lobes, and kept in a round cell culture dish (ThermoFisher) on ice. For tumor-bearing lungs, tumors were separated from healthy tissue using forceps and scissors under a dissecting microscope, and the dissected tumors and surrounding tissue were kept in 5 mL Eppendorf tubes (Sigma Aldrich) for preparation into single-cell suspension. Tissue was minced using Noyes spring scissors (Fine Science Tools) until pieces were less than 1 cm in size, with the visual appearance of ground meat. Minced tissue was then treated with digestion buffer, comprised of Hank's Balanced Salt Solution (HBSS) without Ca²⁺, Mg²⁺ (ThermoFisher) with 2% (v/v) heat-inactivated fetal bovine serum (FBS), supplemented with DNase (40 U/mL, Sigma Aldrich) and collagenase (0.5 mg/mL, Sigma Aldrich). Samples were kept on ice during preparation, and subsequently incubated at 37 C for 30 minutes with end-over-end rotation. Samples were filtered using a 70 μm filter and diluted with RPMI-1640 (ThermoFisher)+2% heat-inactivated FBS. Cell suspension was centrifuged at 625 g for 5 minutes and the pellet was resuspended in ACK lysis buffer (ThermoFisher) for 2 minutes, followed by quenching with FACS buffer (PBS+2% (v/v) heat-inactivated FBS). Cell suspension was centrifuged and supernatant was discarded.

For single cell RNA-seq, CD45+ cell depletion and viability enrichment was performed according to manufacturer's instructions (StemCell Technologies). For depletion of CD45+ cells, the EasySep™ Mouse CD45 Positive Selection Kit (StemCell Technologies), together with a magnet for holding round-bottom or conical tubes (StemCell Technologies), was used for immunomagnetic positive selection of CD45+ leukocytes from the lung tissue preparation, with the goal of ultimately discarding isolated CD45+ cells. Briefly, target CD45+ cells were labeled with antibodies and magnetic particles, and then separated using the magnet. The supernatant suspension containing unlabeled (i.e., desired CD45− cells) were subsequently transferred into a fresh, clean tube. For viability enrichment, the EasySep™ Dead Cell Removal (Annexin V) Kit (StemCell Technologies), together with a magnet for holding round-bottom or conical tubes (StemCell Technologies), was used for column-free immunomagnetic depletion of apoptotic cells from the lung tissue preparation. Briefly, unwanted apoptotic cells were labeled with Annexin V, antibodies, and magnetic particles. Labeled cells were then magnetically separated from the remainder of the suspension, preserving desired cells that were subsequently transferred into a fresh, clean tube. Approximately 70% of cells from Eml4-Alk and healthy lungs were alive following viability enrichment. Following depletion of CD45+ cells and viability enrichment, FACS sorting was not performed prior to single cell RNA-seq.

### Activity-Based Cell Sorting

Single cell lung suspensions from Eml4-Alk mice administered QZ1 were stained with the following antibodies (catalog number, vendor, clone, fluorophore, dilution): CD44 (563508, BD, IM7, BV605, 1:200), CD105 (564746, BD, MJ7/18, BV786, 1:200), Ly6-A/E (12-5981-81, ThermoFisher, D7, PE, 1:200), CD11b (557657, BD, M1/70, APC-Cy7, 1:200), CD45 (566439, BD, 30-F11, AF488, 1:400), and EpCAM (118216, BioLegend™, G8.8, PE-Cy7, 1:200). Cells were stained for 20 minutes, and DAPI (1:10,000) was added immediately prior to sort. FACS sorting was performed on a FACSAria™ II (BD). Flow cytometry data was analyzed by the FlowJo software (Treestar). At least 100,000 cells from each of the QZ1+ and QZ1− compartments were collected into RPMI-1640+2% heat-inactivated FBS and pelleted via centrifugation at 300 g for 5 minutes. Cell pellets were lysed in Trizol™ (ThermoFisher), and RNA was extracted using RNEasy™ Mini Kits (Qiagen). Bulk RNA sequencing was performed by the MIT BioMicro Center. Libraries were prepared using the Clontech SMARTer™ Stranded Total RNAseq™ Kit (Clontech), precleaned, and sequenced using an Illumina NextSeq™500 on an Illumina NextSeq™ flow cell. Feature counting was performed on BAM files using the Rsubread package in R (v4). Differential expression analysis on QZ1+vs QZ1− cells was performed using the DESeq2 package in R (v4). GSEA was performed using the clusterProfiler package and visualized using the enrichplot package in R (v4).

### Analysis of Em14-Alk Bulk RNA-Seq Dataset

Differential expression analysis over the entire transcriptome was performed on a bulk RNA-seq dataset from the Eml4-Alk mouse model of NSCLC, reported by Li et al. [26], using the DESeq2 package in R (v4). The gene list was subsequently filtered to protease genes for visualization.

### RNA-Seq of Eml4-Alk Alveolar Organoids

Alveolar type 2 (AT2) organoids were derived from Trp53^{fl/fl}Rosa26^{LSL-Cas9-2A-eGFP/+} (N=2) and Trp53^{fl/fl}Rosa26^{LSL-tdTomato/+} (N=1) mice. All source mice were females on a C57BL/6 background, with source Rosa26 and Trp53 strains acquired from Jackson Labs. Mice were between 8 and 15 weeks of age at the time of organoid derivation. Organoids were generated according to the protocol described in [39]. These organoid lines were then infected with an adenovirus expressing Cre recombinase (Ad5-CMV-Cre) to generate Trp53-deficient, TdTomato-expressing (PT) and Trp53-deficient, Cas9-expressing (PC) organoids [39]. The Eml4-Alk (EA) inversion was induced in PT organoids using an adenovirus expressing sgRNAs targeting the EA inversion breakpoints and also expressing Cas9 [25]. On the other hand, PC organoids were treated with a lentivirus expressing the same sgRNAs and Cre recombinase. PT Eml4-Alk (PTEA) and PC Eml4-Alk (PCEA) cultures were then incubated in media lacking FGF7, HGF, and NOGGIN to enrich for EA mutant cells. Whole RNA was then extracted from PTEA, PCEA, and two PC cultures (grown in full media) using phenol-chloroform extraction with TRIzol (Invitrogen), followed by purification with a Qiagen RNAeasy MinElute Cleanup Kit. RNA purity was determined by UV-Vis spectrophotometry (NanoDrop™) and all samples exhibited 260/280 ratios of greater than 1.98. Bulk RNA sequencing was performed by the MIT BioMicro Center. Libraries were sequenced using an Illumina NextSeq™500 on an Illumina NextSeq™ flow cell with 75 nt read lengths. Feature counting was performed on BAM files using the Rsubread package in R (v4). Differential expression analysis on Eml4-Alk-mutant vs. control PC organoids was performed using the DESeq2 package in R (v4). GSEA was performed using the clusterProfiler package and visualized using the enrichplot package in R (v4).

Single Cell RNA Sequencing (scRNA-Seq)

Following preparation of the single-cell suspension after depletion of CD45+ cells and viability enrichment, single cells were processed using the 10× Genomics Single Cell 3′ platform using the Chromium Single Cell 3′ Library & Gel Bead Kit V2 kit (10× Genomics), per manufacturer's protocol. Briefly, approximately 10,000 cells were loaded onto each channel and partitioned into Gel Beads in Emulsion (GEMs) in the 10× Chromium instrument. No FACS sorting was performed prior to loading on the 10× Chromium instrument. Following lysis of the captured cells, the released RNA was barcoded through reverse transcription in individual GEMs, and complementary DNA was generated and amplified. Libraries were constructed using a Single Cell 3′ Library and Gel Bead kit. The libraries were sequenced using an Illumina NovaSeq6000 sequencer on an Illumina NovaSeq™ SP flow cell with a read length of 100 nucleotides. scRNA-seq was performed by the MIT BioMicro Center.

### Single Cell RNA-Seq Data Analysis

Raw gene expression matrices were generated for each sample by the Cell Ranger (v.3.0.2) Pipeline coupled with mouse reference version GRCm38. The output filtered gene expression matrices were analyzed by Python software (v.3.9.0) with the Scanpy™ package (v.1.7.2) [55]. The mean sequencing depth (mean number of raw reads per cell) was 9,727 reads per cell for the Eml4-Alk lungs dataset and 15,929 reads per cell for the healthy lungs dataset. Genes expressed in at least three cells in the data and cells with >200 genes detected were selected for further analyses. Low quality cells were removed based on the number of total counts and percentage of mitochondrial genes expressed. Specifically, cells with fewer than 4000 genes per cell (approximately <15,000 counts per cell) and less than 5% mitochondrial genes were retained, with thresholds selected based on the distribution of genes per cell vs. library size and the distribution of the percentage of counts in mitochondrial genes vs. library size. After removal of low quality cells, gene count matrices were total-count normalized, i.e. library-size normalized, to correct for library size, such that counts became comparable across cells. The gene counts for each cell were normalized by total counts over all genes with a scaling factor of 10,000, such that every cell had the same total of 10,000 after normalization. Normalized counts were log transformed (i.e., log(1+x) where x is the number of counts) to stabilize variance and facilitate comparison of relative differences in gene expression. The dataset was additionally filtered to remove cells expressing Ptprc (CD45). Features with high cell-to-cell variation were calculated. Principal component analysis (PCA) was conducted on highly variable genes using the scanpy.tl.pca function with default parameters on normalized and scaled data. A k-nn neighborhood graph was computed over the PCA representation of the data, using the scanpy.pp.neighbors function with default parameters. The neighborhood graph was subsequently embedded via uniform manifold approximation and projection (UMAP) for dimensionality reduction, and cells were clustered in the UMAP embedding space using the Louvain algorithm with resolution 0.25. Cell types were annotated based on expression of known lung cell type marker genes (Table 5) curated from the literature [34, 35]. All analyses and visualizations were implemented in Python with support from Scanpy™ [55].

### Statistics and Reproducibility

Differential gene expression analysis for bulk RNA-seq data was performed in R. PCA and machine learning classification of activity-based nanosensor data was performed in Python (v.3.9.0) using the Protease Activity Analysis (PAA) package. scRNA-seq data analysis was performed in Python (v.3.9.0) using the Scanpy™ (v.1.7.2) package [55]. All remaining statistical analyses were conducted in Prism™ 9.0 (GraphPad). Sample sizes, statistical tests, and p-values are specified in figure legends.

Activity-based nanosensor, scRNA-seq, Eml4-Alk organoid, and activity-based cell sorting experiments were repeated twice with similar results. All other experiments (including AZP, immunohistochemistry, and immunofluorescence staining experiments) were repeated three times with similar results. Details on the reproducibility of representative images are provided in the relevant figure legends.

### REFERENCES

[1] Hanahan, D. & Weinberg, R. A. Hallmarks of cancer: The next generation. Cell 144, 646-674 (2011).
[2] Letai, A. Functional precision cancer medicine-moving beyond pure genomics. Nature Medicine 23, 1028-1035 (2017).
[3] Karczewski, K. J. & Snyder, M. P. Integrative omics for health and disease. Nature Reviews Genetics 19, 299-310 (2018).
[4] Patel, A. P. et al. Single-cell rna-seq highlights intratumoral heterogeneity in primary glioblastoma. Science 344, 1396-1401 (2014).
[5] Navin, N. E. The first five years of single-cell cancer genomics and beyond. Genome research 25, 1499-1507(2015).
[6] Stegle, O., Teichmann, S. A. & Marioni, J. C. Computational and analytical challenges in single-cell transcriptomics. Nature Reviews Genetics 16, 133-145 (2015).
[7] Pon\acute{t}n, F. et al. A global view of protein expression in human cells, tissues, and organs. Molecular systems biology 5, 337 (2009).
[8] Goltsev, Y. et al. Deep profiling of mouse splenic architecture with codex multiplexed imaging. Cell 174, 968-981 (2018).
[9] Lee, J. H. et al. Highly multiplexed subcellular ma sequencing in situ. Science 343, 1360-1363 (2014).
[10] Chen, K. H., Boettiger, A. N., Moffitt, J. R., Wang, S. & Zhuang, X. Spatially resolved, highly multiplexed ma profiling in single cells. Science 348 (2015).
[11] StAhl, P. L. et al. Visualization and analysis of gene expression in tissue sections by spatial transcriptomics. Science 353, 78-82 (2016)
[12] Lewis, S. M. et al. Spatial omics and multiplexed imaging to explore cancer biology. Nature Methods 1-16 (2021).
[13] Weissleder, R., Tung, C. H., Mahmood, U. & Bogdanov, A. In vivo imaging of tumors with protease-activated near-infrared fluorescent probes. Nature Biotechnology 17, 375-378 (1999).
[14] Blum, G., Von Degenfeld, G., Merchant, M. J., Blau, H. M. & Bogyo, M. Noninvasive optical imaging of cysteine protease activity using fluorescently quenched activity-based probes. Nature Chemical Biology 3, 668-677 (2007).
[15] Kwong, G. A. et al. Mass-encoded synthetic biomarkers for multiplexed urinary monitoring of disease. Nature Biotechnology 31, 63-70 (2013).
[16] Lakshmanan, A. et al. Acoustic biosensors for ultrasound imaging of enzyme activity. Nature chemical biology 16, 988-996 (2020).
[17] Soleimany, A. P. & Bhatia, S. N. Activity-Based Diagnostics: An Emerging Paradigm for Disease Detection and Monitoring. Trends in Molecular Medicine 26, 450-468 (2020).
[18] Kwong, G. A. et al. Synthetic biomarkers: a twenty-first century path to early cancer detection. Nature Reviews Cancer 1-14 (2021).
[19] Anahtar, M. et al. Host protease activity classifies pneumonia etiology. Proceedings of the National Academy of Sciences 119, e2121778119 (2022).
[20] Kwon, E. J., Dudani, J. S. & Bhatia, S. N. Ultrasensitive tumour-penetrating nanosensors of protease activity. Nature Biomedical Engineering 1, 1-10 (2017).
[21] Loynachan, C. N. et al. Renal clearable catalytic gold nanoclusters for in vivo disease monitoring. Nature Nanotechnology 14, 883-890 (2019).
[22] Kirkpatrick, J. D. et al. Urinary detection of lung cancer in mice via noninvasive pulmonary protease profiling. Science Translational Medicine 12, eaaw0262 (2020).
[23] Hao, L. et al. Microenvironment-triggered multimodal precision diagnostics. Nature Materials 1-9 (2021).
[24] Walker-Samuel, S. et al. In vivo imaging of glucose uptake and metabolism in tumors. Nature medicine 19, 1067-1072 (2013).
[25] Maddalo, D. et al. In vivo engineering of oncogenic chromosomal rearrangements with the CRISPR/Cas9 system. Nature 516, 423-428 (2014).
[26] Li, X. et al. High-resolution in vivo identification of miRNA targets by halo-enhanced Ago2 pull-down. Molecular Cell 79, 167-179 (2020).
[27] Peters, S. et al. Alectinib versus crizotinib in untreated ALK-positive non-small-cell lung cancer. New England Journal of Medicine 377, 829-838 (2017).
[28] Soleimany, A. P. et al. Activatable zymography probes enable in situ localization of protease dysregulation in cancer. Cancer Research 81, 213-224 (2021).
[29] Dudani, J. S., Ibrahim, M., Kirkpatrick, J., Warren, A. D. & Bhatia, S. N. Classification of prostate cancer using a protease activity nanosensor library. Proc. Natl. Acad. Sci. U.S.A. 115, 8954-8959(2018).
[30] Vestweber, D. VE-cadherin: the major endothelial adhesion molecule controlling cellular junctions and blood vessel formation. Arterioscler. Thromb. Vasc. Biol. 28, 223-232(2008).
[31] Armulik, A., Genové, G. & Betsholtz, C. Pericytes: developmental, physiological, and pathological perspectives, problems, and promises. Developmental Cell 21, 193-215 (2011).
[32] Raza, A., Franklin, M. J. & Dudek, A. Z. Pericytes and vessel maturation during tumor angiogenesis and metastasis. American Journal of Hematology 85, 593-598 (2010).
[33] Armulik, A., Abramsson, A. & Betsholtz, C. Endothelial/pericyte interactions. Circulation Research 97, 512-523 (2005).
[34] Tabula Muris Consortium. Single-cell transcriptomics of 20 mouse organs creates a Tabula Muris. Nature 562, 367-372 (2018).
[35] Travaglini, K. J. et al. A molecular cell atlas of the human lung from single-cell RNA sequencing. Nature 587, 619-625 (2020).
[36] Andrae, J., Gallini, R. & Betsholtz, C. Role of platelet-derived growth factors in physiology and medicine. Genes & Development 22, 1276-1312 (2008).
[37] Lindblom, P. et al. Endothelial PDGF-B retention is required for proper investment of pericytes in the microvessel wall. Genes & Development 17, 1835-1840 (2003).
[38] Song, N. et al. Overexpression of platelet-derived growth factor-BB increases tumor pericyte content via stromal-derived factor-1α/CXCR4 axis. Cancer Research69, 6057-6064(2009).
[39] Naranjo, S. et al. Modeling diverse genetic subtypes of lung adenocarcinoma with a next-generation alveolar type 2 organoid platform. bioRxiv (2021).
[40] Jiang, T. et al. Tumor imaging by means of proteolytic activation of cell-penetrating peptides. Proc. Natl. Acad. &i. U.S.A. 101, 17867-17872 (2004).
[41] Savariar, E. N. et al. Real-time in vivo molecular detection of primary tumors and metastases with ratiometric activatable cell-penetrating peptides. Cancer Research 73, 855-864 (2013).
[42] Santos, A. M., Jung, J., Aziz. N., Kissil, J. L. & Puré, E. Targeting fibroblast activation protein inhibits tumor stromagenesis and growth in mice. The Journal of Clinical Investigation 119, 3613-3625 (2009).
[43] Chen, M. et al. Pericyte-targeting prodrug overcomes tumor resistance to vascular disrupting agents. The Journal of Clinical Investigation 127, 3689-3701 (2017).
[44] Ferrara, N. Hillan, K. J., Gerber, H.-P. & Novotny. W. Discovery and development of bevacizumab, an anti-VEGF antibody for treating cancer. Nature Reviews Drug discovery 3, 391-400 (2004).
[45] Sandler, A. et al. Paclitaxel-carboplatin alone or with bevacizumab for non-small-cell lung cancer. New England. Journal of Medicine 355, 2542-2550 (2006).
[46] Socinski, M. A. et al. Atezolizumab for first-line treatment of metastatic nonsquamous NSCLC. New England Journal of Medicine 378, 2288-2301 (2018).
[47] Wilson, D. S., Keefe, A. D.& Szostak, J. W. The use of mRNA display to select high-affinity protein-binding peptides. Proc. Natl. Acad. Sci. U.S.A. 98, 3750-3755 (2001).
[48] O'Donoghue, A. J. et al. Global identification of peptidase specificity by multiplex substrate profiling. Nature Methods 9, 1095-1100 (2012).
[49] Poreba, M., Salvesen, G. S. & Drag, M. Synthesis of a HyCoSuL peptide substrate library to dissect protease substrate specificity. Nature Protocols 12, 2189-2214 (2017).
[50] Muller, A. T., Hiss, J. A. & Schneider, G. Recurrent neural network model for constructive peptide design. Journal of Chemical Information and Modeling 58, 472-479 (2018).
[51] DuPage, M., Dooley, A. L. & Jacks, T. Conditional mouse lung cancer models using adenoviral or lentiviral delivery of cre recombinase. Nature protocols 4, 1064-1072 (2009).
[52] Soleimany, A. P., Martin-Alonso, C., Anahtar, M., Wang, C. S. & Bhatia, S. N. Protease activity analysis: A toolkit for analyzing enzyme activity data. A CS Omega (2022).
[53] Bankhead, P. et al. QuPath: Open source software for digital pathology image analysis. Scientific Reports 7, 1-7 (2017).
[54] Bolte, S. & Cordelières, F. P. A guided tour into subcellular colocalization analysis in light microscopy. Journal of Microscopy 224, 213-232 (2006).
[55] Wolf, F. A., Angerer, P. & Theis, F. J. SCANPY: large-scale single-cell gene expression data analysis. Genome Biology 19, 1-5 (2018)

### Example 2

### Ex Vivo Assays for Multiplexed and Spatially Resolved Enzyme Activity Profiling

Protease activity is dysregulated across multiple disease states, including cancer, fibrosis, and infection. Here, we have developed “activatable zymography probes” (AZPs) that can measure and localize protease activity in tissue sections. As a proof-of-concept, we applied our assays to prostate cancer (PCa), a disease whose management would benefit from “smart” diagnostics and therapeutics that specifically identify and target aggressive disease. We demonstrate that AZPs are able to bind, in a protease-dependent manner, to regions of elevated protease activity in normal and diseased mouse tissue. We then leverage clinically sourced human PCa biopsy samples and discover two AZPs that preferentially label PCa tissue over normal prostate tissue. We envision that these modular tools will facilitate design of protease-activatable diagnostics and therapeutics.

We sought to develop probes to map the location of proteolytic cleavage events in situ in order to 1) better understand and localize the source of protease activity in the tissue microenvironment and 2) inform the development of conditionally activated diagnostics and therapeutics. We hypothesized that peptides consisting of a cationic (poly-arginine, or polyR) domain bound to an anionic (poly-glutamic acid, or polyE) domain via a linker would remain complexed as long as the linker remained intact. By incorporating protease recognition and cleavage sites into the linker, we hypothesized that protease activity could be used to drive binding of the polycationic polyR domain to negatively charged tissue regions. In theory, this could enable in situ localization of protease activity.

We validated the AZP principle on colon tissue, whose epithelial cells secrete serine proteases including urokinase plasminogen activator (uPA). We therefore synthesized a Cy5-labeled AZP, termed PZ2 (SEQ ID NO:176), containing a serine protease-cleavable linker. Because similar peptides had never been applied directly to frozen tissue sections, it was unknown whether AZPs would nonspecifically bind to the tissue, or whether proteolytic cleavage would be required for AZP binding. We therefore pre-cleaved PZ2 with a serine protease, mesotrypsin (PRSS3) and applied it to a frozen section of mouse colon for 30 minutes. As a binding control, we also applied intact, uncleaved PZ2 to a consecutive section of mouse colon. Though minimal fluorescence was observed on colons treated with intact PZ2, colons treated with pre-cleaved PZ2 exhibited notable fluorescent signal throughout (FIG. 18).

Having verified that proteolytic cleavage induces AZP binding, we next sought to validate whether AZPs could enable localization of protease activity in situ (FIG. 19a). Specifically, it was unknown whether frozen tissue sections would maintain sufficient protease activity to induce probe cleavage or whether cleaved probes would bind at the site of cleavage rather than diffusing to a distant site. We applied PZ2 and a binding control (free polyR-Cy7) to fresh-frozen sections of mouse colon (FIG. 19b). After a four-hour incubation, we observed strong PZ2 staining (FIG. 19c, top) that was abrogated by addition of the serine protease inhibitor 4-(2-aminoethyl)benzenesulfonyl fluoride hydrochloride (AEBSF; FIG. 19c, middle). Furthermore, no Cy5 fluorescence was observed after incubation with dPZ2, an AZP with a non-cleavable d-stereoisomer linker (FIG. 19c, bottom). These findings verified that protease activity in the tissue section was necessary and sufficient to activate AZPs. Strikingly, we found strong colocalization of PZ2 with E-cadherin, a specific marker of epithelial cells, providing evidence that cleaved AZPs bind at the site of cleavage, rather than diffusing to a distant site (FIG. 19d). Together, these results indicated that AZPs could directly measure peptide cleavage events in situ to spatially localize protease activity with low background binding.

We next sought to demonstrate the utility of AZPs in discovering disease-specific peptides and localizing their degradation in situ. We turned to the Hi-Myc genetically engineered mouse model of PCa, wherein c-Myc is overexpressed in the murine prostate, resulting in invasive PCa. We selected a panel of 18 peptides previously found by our group to be recognized by a diverse array of metallo- and serine proteases dysregulated in PCa²⁰ and appended each with a unique, mass-encoded reporter molecule. We coupled these barcoded substrates to magnetic beads via a non-cleavable poly(ethylene glycol) (PEG) linker and incubated the bead cocktail against homogenates of prostates from PCa mice and from age-matched healthy controls (FIG. 20a). Beads were pulled down via magnetic separation, and liberated reporter molecules in the supernatant were measured by mass spectrometry, enabling quantification of substrate cleavage. Unsupervised hierarchical clustering of the cleavage data yielded two distinct clusters corresponding to prostates from PCa mice and prostates from healthy mice (FIG. 20b).

Surprisingly, this assay reveled differential cleavage of a single peptide, PM19, in Hi-Myc prostates relative to healthy controls (FIG. 20c). Based on previous results from a screen of this peptide against recombinant proteases²⁰, we hypothesized that MMPs were contributing most significantly to PM19 cleavage. Consistent with this hypothesis, we found that pre-treatment of the prostate homogenates with the MMP inhibitor marimastat (MAR) fully inhibited cleavage of PM19 in the Hi-Myc prostates (FIG. 20d; P<0.0001). In contrast, the serine protease inhibitor AEBSF minimally affected cleavage of this tumor-specific substrate.

It was unknown whether AZPs could enable in situ localization of the cleavage of peptides discovered in a screen of bulk homogenates. We therefore synthesized a new AZP (PZ19: SEQ ID NO:186) and applied it to fresh-frozen sections of prostates from Hi-Myc mice (FIG. 21a). With the exception of the protease-cleavable linker and fluorophore, PZ19 was identical in design to the serine protease-responsive PZ2. Intriguingly, this assay revealed PZ19 labeling in histologically normal glands of the Hi-Myc prostate (FIG. 21a inset; FIG. 21b top row). PZ19 labeling was abrogated by the MMP inhibitor marimastat (FIG. 21b middle row), though the free polyR control was unchanged. Furthermore, no tissue labeling was observed in sections treated with the non-cleavable, d-stereoisomer version of the probe, dPZ19, further validating that PZ19 labeling was proteolytically driven (FIG. 21b).

The discovery that PZ19 localized to histologically normal prostatic glands suggested that these may represent early neoplastic lesions and motivated further biological characterization of their proliferative capacity. It was unknown whether AZPs could be used in conjunction with antibodies to enable dual zymography and immunofluorescence labeling. However, when we applied PZ19 to prostate tissue sections from Hi-Myc and age-matched healthy mice and co-stained for the proliferation marker Ki67, we observed striking Ki67 staining that co-localized with activated PZ19 in this region of Hi-Myc prostate (FIG. 21a), but no Ki67 staining and reduced PZ19 labeling in a histologically similar region of normal mouse prostate (FIG. 21c, d). Further, PZ19 labeling and co-localization with Ki67 was abrogated by addition of marimastat (FIG. 21d). Taken together, these measurements revealed that AZPs enable localization of peptide cleavage events discovered by bulk substrate screening, and can be used in conjunction with immunofluorcscence to provide new biological insights that cannot be obtained with either method alone.

Motivated by these results, we next sought to identify candidate MMPs dysregulated in the Hi-Myc model as potential protease targets for PZ19. We queried previously reported gene expression data from the Hi-Myc model¹⁹ and found that the elastase MMP12, which cleaves the substrate incorporated in PZ19, was significantly overexpressed in Hi-Myc PCa relative to healthy prostate tissue. To validate this transcript on the protein level, we measured MMP12 abundance in homogenates of Hi-Myc prostates and of age-matched healthy controls, and found a significant increase in MMP12 levels in the Hi-Myc tissues (P=0.0034). Additionally, Hi-Myc prostates stained positively for MMP12 while healthy prostates did not. Finally, we asked whether MMP12 localized to the PZ19, Ki67-positive region of Hi-Myc tissue (FIG. 21), and observed consistent MMP12 staining in this region. These results provided further evidence that AZP binding localizes to regions of increased protease activity.

However, it remained uncertain whether AZPs could be used as a method to discover disease-specific peptide substrates in clinically relevant human tissue samples. Furthermore, the modularity of the AZP platform was unknown. We first asked whether we could interchange the peptide linker with any desired substrate. We designed a library of AZPs based on a panel of peptides previously found by our group to be recognized by proteases dysregulated in PCa²⁰. We first sought to characterize these AZPs in regards to their i5 proteolytic responsiveness and tissue binding. AZPs, either intact or with linkers pre-cleaved by a cognate recombinant protease dysregulated in PCa (MMP13 for MMP-responsive substrates; PRSS3, KLK14, or KLK2 for serine protease-responsive substrates), were incubated with fresh frozen colon tissue, and probe binding was assessed by fluorescence microscopy after washing away unbound peptides. Given their widely ranging hydrophobicities, steric, and electrostatic properties, we were surprised to find that every tested AZP exhibited increased tissue binding following pre-cleavage (FIG. 22), demonstrating the modularity of the method.

Having validated that proteolytic cleavage significantly enhanced AZP binding, we next selected a subset of the AZP library to evaluate against a fresh-frozen human PCa tissue microarray (TMA) for analysis of in situ protease activity, with the aim of identifying peptides that were selectively cleaved in human PCa. Two AZPs incorporating MMP-responsive substrates, PZ16 (SEQ ID NO:184) and PZ19, and two AZPs incorporating serine protease-responsive substrates, Z2 (SEQ ID NO:172) and PZ11 (SEQ ID NO:180), were selected for evaluation. We applied these probes, along with a free polyR control, to a frozen human PCa TMA that contained biopsies from normal prostates and PCa tumors across a range of Gleason scores.

To determine whether AZP binding was dependent on proteolysis, for each probe a consecutive TMA section was treated with a broad-spectrum cocktail of protease inhibitors. Surprisingly, no difference between the uninhibited and inhibited conditions was observed for PZ19 (FIG. 23a), demonstrating the importance of testing in human tissue rather than in mouse models alone. In contrast, however, the AZPs PZ16 (FIG. 23b), PZ11 (FIG. 24a, b), and Z2 (FIG. 24c, d) exhibited protease-dependent labeling of the PCa TMA, as labeling was abrogated by addition of a broad-spectrum cocktail of protease inhibitors.

We then investigated whether AZPs preferentially labeled PCa tissue relative to normal prostates. While no significant difference between PCa and normal cores was observed for PZ19 and PZ16 (FIG. 25), PZ11 (FIG. 26a) and Z2 (FIG. 26d) were found to preferentially label PCa tissue. AZP activation and tissue staining across the TMA was quantitatively assessed by normalizing AZP signal intensity to polyR signal to account for differing binding propensities within a single core and across individual specimens. This quantification revealed that the serine protease-responsive AZPs PZ11 (FIG. 26b) and Z2 (FIG. 26c) were significantly enriched in PCa relative to normal prostate tissue. We further assessed the classification accuracy of these two in situ probes using receiver operating characteristic (ROC) curves. We found that PZ11 and Z2 enabled accurate disease classification with areas under the curve (AUC) of 0.948 (FIG. 26c) and 0.917 (FIG. 26f), respectively. These findings demonstrate that serine protease activity is dysregulated in human PCa and that AZPs may be used to discover disease-responsive peptide substrates.

### Activatable Zymography Probes are Targeting Independent

In situ zymography (ISZ) assays aim to enable visualization of protease activity in tissue sections. Though early ISZ worked centered on visualizing the cleavage of full-length proteins (e.g. gelatin), recent efforts have aimed to expand the modularity of ISZ with synthetic peptides. However, existing methods have relied on expression of integrins or receptors (e.g., EGFR) to tether probes to the tissue. This reliance on targeting means that the readout is a convolution of proteolytic activity and target binding, which restricts the generalizability of these methods. In contrast, AZPs are targeting independent, thereby providing a pure readout of enzyme activity.

### Activatable Zymography Probes are Highly Modular

AZPs were designed to be modular so that cationic polypeptide release, exemplified by poly-arginine (polyR), can be tuned to be responsive to specific proteases via modification of the peptide linker sequence. Because AZP activation measures proteolytic cleavage, rather than covalent binding to the enzyme active site, AZPs can readily be designed to query activities of proteases from all catalytic classes, including matrix metalloproteinases (MMPs). In contrast, activity-based probes (ABPs) rely on covalent binding of a chemical warhead to the enzyme active site, which severely limits their utility for MMP activity profiling. A cleavage-dependent readout, as presented here, overcomes this limitation and provides a modular means for in situ activity profiling of proteases from all catalytic classes. Use of orthogonal fluorophores, affinity tags, or DNA barcodes provides a number of possible AZP configurations for multiplexed in situ activity profiling. In addition to a protease-cleavable peptide linker, the polyR and polyE domains may be linked by other classes of enzyme substrates such as glycans, lipids, or nucleic acids. The resulting activatable probes may therefore be used to query glycosidase, lipase, and nuclease activity directly in situ.

### Activatable Zymography Probes can be Used to Discover Disease-Specific Peptides for Conditionally Activated Diagnostics and Therapeutics.

We have demonstrated that AZPs can be screened against clinically accessible human tissue (frozen biopsy cores) to identify peptides that are preferentially cleaved by PCa tissue. In theory, a protease activated therapeutic (e.g., probody) incorporating the PZ11 sequence “GIQQRSLGGG” (SEQ ID No:227) or the Z2 sequence “GGLVPRGSG” (SEQ ID No:228) could offer higher on-target (i.e., PCa) and less off-target (i.e., normal prostate) selectivity compared to an unmasked therapeutic. Gelatin-based in situ zymography cannot inform development of conditionally activated diagnostics and therapeutics because an endogenous, full-length protein is used, rather than a short peptide. AZP multiplexing with epitope tags or DNA barcodes could enable high-throughput screening for cancer-specific peptides directly on human tissue sections.

Activatable zymography probes (AZPs) would be extremely valuable in identification of candidate peptide substrates for incorporation into protease-activatable diagnostics and therapeutics.

### REFERENCES

1. Weissleder, R., Tung, C.-H., Mahmood, U. & Bogdanov, A. In vivo imaging of tumors with protease-activated near-infrared fluorescent probes. Nat. Biotechnol. (1999). doi:10.1038/7933
2. Blum, G., Von Degenfeld, G., Merchant, M. J., Blau, H. M. & Bogyo, M. Noninvasive optical imaging of cysteine protease activity using fluorescently quenched activity-based probes. doi:10.1038/nchembio.2007.26
3. Sanman, L. E. & Bogyo, M. Activity-Based Profiling of Proteases. Annu. Rev. Biochem. 83, 249-273 (2014).
4. Desnoyers, L. R. et al. Tumor-specific activation of an EGFR-targeting probody enhances therapeutic index. Sci. Transl. Med. 5, (2013).
5. Polu, K. R. & Lowman, H. B. Probody therapeutics for targeting antibodies to diseased tissue. Expert Opin. Biol. Ther. 14, 1049-1053 (2014).
6. Chen, I. J. et al. Selective antibody activation through protease-activated pro-antibodies that mask binding sites with inhibitory domains. Sci. Rep. 7, 1-12 (2017).
7. Trang, V. H. et al. A coiled-coil masking domain for selective activation of therapeutic antibodies. Nat. Biotechnol. 37, 761-765 (2019).
8. Wang, D. et al. Engineering nanoparticles to locally activate T cells in the tumor microenvironment. Sci. Immunol. 4, eaau6584 (2019).
9. Withana. N. P. et al. Non-invasive Imaging of Idiopathic Pulmonary Fibrosis Using Cathepsin Protease Probes. (2015). doi:10.1038/srep19755
10. Nguyen, Q. T. et at. Surgery with molecular fluorescence imaging using activatable cell-penetrating peptides decreases residual cancer and improves survival. Proc. Natl. Acad. Sci. U.S.A 107, 4317-22 (2010).
11. Olson, E. S. et al. Activatable cell penetrating peptides linked to nanoparticles as dual probes for in vivo fluorescence and MR imaging of proteases. Proc. Natl. Acad. Sc. U.S.A. 107, 4311-6 (2010).
12. Whitley, M. J. et at. A mouse-human phase 1 co-clinical trial of a protease-activated fluorescent probe for imaging cancer. Sci. Transl. Med. 8, (2016).
13. Kwong, G. A. et al. Mass-encoded synthetic biomarkers for multiplexed urinary monitoring of disease. Nat. Biotechnol. 31, (2012).
14. Dudani, J. S., Warren, A. D. & Bhatia, S. N. Harnessing Protease Activity to Improve Cancer Care. Annu. Rev. Cancer Biol. 2, 353-376 (2018).
15. Prensner, J. R., Rubin, M. A., Wei, J. T. & Chinnaiyan, A. M. Beyond PSA: The next generation of prostate cancer biomarkers. Sci. Transl. Med. 4, (2012).
16. Jiang, T. et at. Tumor imaging by means of proteolytic activation of cell-penetrating peptides. (2004).
17. Vergnolle, N. Protease inhibition as new therapeutic strategy for GI diseases. Gut 65, 1215-1224 (2016).
18. Uhlén, M. et al. Tissue-based map of the human proteome. Science 347, (2015).
19. Ellwood-Yen, K. et al. Myc-driven murine prostate cancer shares molecular features with human prostate tumors. Cancer Cell 4, 223-238 (2003).
20. Dudani. J. S., Ibrahim, M., Kirkpatrick, J., Warren, A. D. & Bhatia, S. N. Classification of prostate cancer using a protease activity nanosensor library. Proc. Natl. Acad. Sci. U.S.A. 115, 8954-8959 (2018).
21. Vandooren, J., Geurts, N., Martens. E. & Steen, P. E. Van Den. Zymography methods for visualizing hydrolytic enzymes. 10, 211-220 (2013).
22. Kwon, E. J., Dudani, J. S. & Bhatia, S. N. Ultrasensitive tumour-penetrating nanosensors of protease activity. Nat. Biomed Eng 1, (2017).

1. A method for activity based cell sorting, comprising
(a) contacting a biological sample with an activatable zymography probe (AZP), wherein the AZP comprises the general formula X1-X2-X3, wherein
(i) one of X1 and X3 is a cationic peptide linked to a fluorophore, and the other is an anionic peptide; and
(ii) X2 is protease-cleavable peptide,
wherein degradation of the protease-cleavable peptide by proteases present in the biological sample liberates the fluorophore-linked cationic peptide so that it binds cells at or near a site of protease degradation of the protease-cleavable peptide to produce fluorophore-tagged cells; and
(b) isolating the fluorophore tagged cells from the biological sample by fluorescence activated cell sorting (FACS).

2. The method of claim 1, wherein the cationic peptide only includes R, K, and/or H residues, and/or wherein the anionic peptide only includes D and/or E residues.

3. (canceled)

4. The method of claim 1, wherein the cationic peptide is a polyR peptide, and/or wherein the anionic peptide is a polyE peptide.

5.-7. (canceled)

8. The method of claim 1, wherein the fluorophore is selected from the group consisting of fluorescein phosphoramidides, rhodamine, polymethadine dye derivative, phosphores, Texas red, green fluorescent protein, Cy3, Cy5, and Cy7.

9. The method of claim 1, wherein the fluorophore is a fluorophore component of a fluorophore-quencher pair, and the anionic peptide linked to the quencher component of the fluorophore-quencher pair.

10.-11. (canceled)

12. The method of claim 1, wherein one of the cationic peptide or the anionic peptide is linked to Cy5 and the other is linked to Cy7.

13. The method of claim 1, wherein the protease-cleavable peptide comprises the sequence selected from the group consisting of SEQ ID NO:1-166.

14.-17. (canceled)

18. The method of claim 1, wherein the AZP comprises
(a) (QSY21)-eeeeeeeee-c(PEG2K)-oGGPQGIWGQG-rrrrrrrrr-k(Cy5) (SEQ ID NO: 167), wherein:
lowercase “amino acid residues are in D amino acid form;
PEG2K: (poly)ethylene-glycol, MW 2000 g/mol; and
o is 5-amino-3-oxopentanoyl or another residue for physical distancing and/or stabilization, or is absent;
(b) an AZP selected from:
(SEQ ID NO: 168)
UeeeeeeeeXGGPQGIWGQGrrrrrrrrrX-k(Cy5);
(SEQ ID NO: 169)
UeeeeeeeeXGGLGPKGQTGGrrrrrrrrrX-k(Cy3);
(SEQ ID NO: 170)
UeeeeeeeeXGILSRIVGGGrrrrrrrrrX-k(FAM);
(SEQ ID NO: 167)
(QSY21)-eeeeeeeee-c(PEG2K)-oGGPQGIWGQG-
rrrrrrrrr-k(Cy5);
wherein lowercase amino acid residues are D-amino acids; PEG2K is (poly)ethylene-glycol, MW 2000 g/mol; “o” is 5-amino-3-oxopentanoyl or another residue for physical distancing and/or stabilization, or is absent; U is succinoyl or is absent; and X is 6-aminohexanoyl or is absent; and/or
(c) an AZP selected from:
(SEQ ID NO: 171)
U-eeeeeeee-X-GGPQGIWGQG-rrrrrrrrr-X-K(Cy3);
(SEQ ID NO: 172)
U-eeeeeeee-X-GGLVPRGSGG-rrrrrrrrr-X-K(Cy5);
(SEQ ID NO: 173)
U-eeeeeeee-X-GGPVGLIGG-rrrrrrrrr-X-K(5FAM);
(SEQ ID NO: 174)
U-eeeeeeee-X-GPLGVRGKG-rrrrrrrrr-X-K(5FAM);
(SEQ ID NO: 175)
U-eeeeeeee-X-GRQRRALEKG-rrrrrrrrr-X-K(Cy3);
(SEQ ID NO: 176)
U-eeeeeeee-X-GSGRSANAG-rrrrrrrrr-X-K(Cy5);
(SEQ ID NO: 177)
U-eeeeeeee-X-GKPISLISSG-rrrrrrrrr-X-K(5FAM);
(SEQ ID NO: 178)
U-eeeeeeee-X-GILSRIVGGG-rrrrrrrrr-X-K(5FAM);
(SEQ ID NO: 179)
U-eeeeeeee-X-GRPKPVE(Nval)WRKG-rrrrrrrrr-X-K
(5FAM);
(SEQ ID NO: 180)
U-eeeeeeee-X-GIQQRSLGGG-rrrrrrrr-X-K(5FAM);
(SEQ ID NO: 181)
U-eeeeeeee-X-GGGVPRGGG-rrrrrrrr-X-K(5FAM);
(SEQ ID NO: 182)
U-eeeeeeee-X-GSGSKIIGGG-rrrrrrrrr-X-K(Cy3);
(SEQ ID NO: 183)
U-eeeeeeee-X-GGAANLTRGG-rrrrrrrrr-X-K(Cy3);
(SEQ ID NO: 184)
U-eeeeeeee-X-GLAQAPhe(homo)RSG-rrrrrrrrr-X-K(Cy3);
(SEQ ID NO: 185)
U-eeeeeeee-X-GSPLAQAVRSSG-rrrrrrrrr-X-K(Cy3);
(SEQ ID NO: 186)
U-eeeeeeee-X-GPVPLSLVMG-rrrrrrrrr-X-K(5FAM);
(SEQ ID NO: 187)
U-eeeeeeee-X-GSQPRIVGGG-rrrrrrrrr-X-K(Cy5);
(SEQ ID NO: 188)
U-eeeeeeee-X-GGGHARLVHVG-rrrrrrrrr-X-K(Cy3);
(SEQ ID NO: 189)
U-eeeeeece-X-GGLGPKGQTGG-rrrrrrrrr-X-K(Cy3);
(SEQ ID NO: 190)
U-eeeeeeee-X-GGQTCKCSCKG-rrrrrrrrr-X-K(Cy5);
(SEQ ID NO: 191)
U-eeeeeeee-X-GsGrsanaG-rrrrrrrrr-X-K(Cy5);
(SEQ ID NO: 192)
(QSY21)-eeeeeeeee-c-o-GSGRSANAG-rrrrrrrrr-K(Cy5);
(SEQ ID NO: 193)
(QSY21)-eeeeeeeee-c-o-GsGrsanaG-rrrrrrrrr-K(Cy5);
(SEQ ID NO: 194)
U-eeeeeeee-X-Gpvp U-eeeeeeee-X-GRQRRALEKG-
rrrrrrrrr-X-K lslvmG-rrrrrrrrr-X-K(5FAM);
(SEQ ID NO: 195)
(QSY21)-eeeeeeeee-c-o-GPVPLSLVMG-rrrrrrrrr-K(Cy5);
(SEQ ID NO: 196)
(QSY21)-eeeeeeeee-c-o-GpvplslvmG-rrrrrrrrr-K(Cy5);
wherein lowercase amino acid residues are D-amino acids; “o” is 5-amino-3-oxopentanoyl or another residue for physical distancing and/or stabilization, or is absent; U is succinoyl or is absent; and X is 6-aminohexanoyl or is absent.

19. (canceled)

20. A probe, comprising the general formula X1-X2-X3, wherein
one of X1 and X3 is a cationic peptide linked to a detectable marker and the other is a anionic peptide;
X2 is a protease-sensitive peptide.

21. The probe of claim 20, wherein the cationic peptide only includes R, K, and/or H residues; and/or wherein the anionic peptide only includes D and/or E residues.

22. (canceled)

23. The probe of any claim 20, wherein cationic peptide is a polyR peptide, and/or wherein the anionic peptide is a polyE peptide.

24.-26. (canceled)

27. The probe of claim 20, wherein the detectable maker is a fluorophore selected from the group consisting of fluorescein phosphoramidides, rhodamine, polymethadine dye derivative, phosphores, Texas red, green fluorescent protein, Cy3, Cy5, and Cy7.

28. The probe of claim 20, wherein the detectable maker is a fluorophore component of a fluorophore-quencher pair, and the anionic peptide is linked to the quencher component of the fluorophore-quencher pair.

29.-30. (canceled)

31. The method of claim 20, wherein one of the cationic peptide or the anionic peptide is linked to Cy5 and the other is linked to Cy7.

32. The probe of claim 20, wherein the protease-sensitive peptide comprises a sequence selected from the group consisting of SEQ ID NO:1-166.

33.-34. (canceled)

35. The probe of claim 20, wherein the cationic domain and/or the anionic domain include D amino acids, or are exclusively D amino acids.

36. The probe of claim 20, wherein
the cationic domain is polyR in D amino acid form;
the anionic domain is polyE in D amino acid form.

37. The probe of claim 20, comprising the structure:
(SEQ ID NO: 168)
UeeeeeeeeXGGPQGIWGQGrrrrrrrrrX-k(Cy5);
(SEQ ID NO: 169)
UeeeeeeeeXGGLGPKGQTGGrrrrrrrrrX-k(Cy3);
(SEQ ID NO: 170)
UeeeeeeeeXGILSRIVGGGrrrrrrrrrX-k(FAM);
(SEQ ID NO: 167)
(QSY21)-eeeeeeeee-c(PEG2K)-oGGPQGIWGQG-
rrrrrrrrr-k(Cy5);
wherein lowercase amino acid residues are D-amino acids; PEG2K is (poly)ethylene-glycol, MW 2000 g/mol; “o” is 5-amino-3-oxopentanoyl; or another residue for physical distancing and/or stabilization, or is absent U is succinoyl or is absent; and X is 6-aminohexanoyl or is absent.

38. The probe of claim 20, comprising the structure:
(SEQ ID NO: 171)
U-eeeeeeee-X-GGPQGIWGQG-rrrrrrrrr-X-K(Cy3);
(SEQ ID NO: 172)
U-eeeeeeee-X-GGLVPRGSGG-rrrrrrrrr-X-K(Cy5);
(SEQ ID NO: 173)
U-eeeeeeee-X-GGPVGLIGG-rrrrrrrrr-X-K(5FAM);
(SEQ ID NO: 174)
U-eeeeeeee-X-GPLGVRGKG-rrrrrrrrr-X-K(5FAM);
(SEQ ID NO: 175)
U-eeeeeeee-X-GRQRRALEKG-rrrrrrrrr-X-K(Cy3);
(SEQ ID NO: 176)
U-eeeeeeee-X-GSGRSANAG-rrrrrrrrr-X-K(Cy5);
(SEQ ID NO: 177)
U-eeeeeeee-X-GKPISLISSG-rrrrrrrrr-X-K(5FAM);
(SEQ ID NO: 178)
U-eeeeeeee-X-GILSRIVGGG-rrrrrrrrr-X-K(5FAM);
(SEQ ID NO: 179)
U-eeeeeeee-X-GRPKPVE(Nval)WRKG-rrrrrrrrr-X-K
(5FAM);
(SEQ ID NO: 180)
U-eeeeeeee-X-GIQQRSLGGG-rrrrrrrr-X-K(5FAM);
(SEQ ID NO: 181)
U-eeeeeeee-X-GGGVPRGGG-rrrrrrrr-X-K(5FAM);
(SEQ ID NO: 182)
U-eeeeeeee-X-GSGSKIIGGG-rrrrrrrrr-X-K(Cy3);
(SEQ ID NO: 183)
U-eeeeeeee-X-GGAANLTRGG-rrrrrrrrr-X-K(Cy3);
(SEQ ID NO: 184)
U-eeeeeeee-X-GLAQAPhe(homo)RSG-rrrrrrrrr-X-K(Cy3);
(SEQ ID NO: 185)
U-eeeeeeee-X-GSPLAQAVRSSG-rrrrrrrrr-X-K(Cy3);
(SEQ ID NO: 186)
U-eeeeeeee-X-GPVPLSLVMG-rrrrrrrrr-X-K(5FAM);
(SEQ ID NO: 187)
U-eeeeeeee-X-GSQPRIVGGG-rrrrrrrrr-X-K(Cy5);
(SEQ ID NO: 188)
U-eeeeeeee-X-GGGHARLVHVG-rrrrrrrrr-X-K(Cy3);
(SEQ ID NO: 189)
U-eeeeeece-X-GGLGPKGQTGG-rrrrrrrrr-X-K(Cy3);
(SEQ ID NO: 190)
U-eeeeeeee-X-GGQTCKCSCKG-rrrrrrrrr-X-K(Cy5);
(SEQ ID NO: 191)
U-eeeeeeee-X-GsGrsanaG-rrrrrrrrr-X-K(Cy5);
(SEQ ID NO: 192)
(QSY21)-eeeeeeeee-c-o-GSGRSANAG-rrrrrrrrr-K(Cy5);
(SEQ ID NO: 193)
(QSY21)-eeeeeeeee-c-o-GsGrsanaG-rrrrrrrrr-K(Cy5);
(SEQ ID NO: 194)
U-eceeceee-X-Gpvp U-eeeeeeee-X-GRQRRALEKG-
rrrrrrrrr-X-K lslvmG-rrrrrrrrr-X-K(5FAM);
(SEQ ID NO: 195)
(QSY21)-eeeeeeeee-c-o-GPVPLSLVMG-rrrrrrrrr-K(Cy5);
(SEQ ID NO: 196)
(QSY21)-eeeeeeeee-c-o-GpvplslvmG-rrrrrrrrr-K(Cy5);
wherein lowercase amino acid residues are D-amino acids; “o” is 5-amino-3-oxopentanoyl or another residue for physical distancing and/or stabilization; or is absent; U is succinoyl or is absent; and X is 6-aminohexanoyl or is absent.

39. A method for localizing protease activity in a tissue section, comprising
(a) contacting a biological tissue section that is not formalin preserved with a probe, comprising the general formula X1-X2-X3, wherein
one of X1 and X3 is a cationic peptide linked to a detectable marker and the other is a anionic peptide;
X2 is a protease-sensitive peptide, wherein proteases present in the biological tissue section cleave the protease-sensitive peptide, resulting in binding of the detectably tagged cationic peptide to the tissue; and
(b) detecting signal from the biological tissue section, wherein the fluorescence indicates where protease activity is present in the biological tissue section.

40. (canceled)
